(19) United States
(12) Patent Application Publication
(10) Pub. No.: US 2024/0158805 A1
(43) Pub. Date: May 16, 2024

(54) Plants With Stem Rust Resistance

(71) Applicants: COMMONWEALTH SCIENTIFIC AND INDUSTRIAL RESEARCH ORGANISATION, Action, Australian Capital Territory (AU); GRAINS RESEARCH AND DEVELOPMENT CORPORATION, Barton, Australian Capital Territory (AU)

(72) Inventors: Rohit Mago, Acton (AU); Linda Tabe, Acton (AU); Wolfgang Spielmeyer, Acton (AU); Peter Dodds, Acton (AU); Jianping Zhang, Acton (AU)

(21) Appl. No.: 18/265,410

(22) PCT Filed: Dec. 8, 2021
(86) PCT No.: PCT/AU2021/051468

§ 371 Date: Jun. 5, 2023

(30) Foreign Application Priority Data
Dec. 9, 2020 (AU) 2020904574

(51) Int. Cl. C12N 15/82; C12Q 1/6895

(52) U.S. Cl. CPC C12N 15/8282; C12N 15/8223; C12Q 1/6895

(57) ABSTRACT

The present invention relates to a genetically modified plant which has enhanced resistance to one or more fungal pathogen(s).

### FIELD OF THE INVENTION

The present invention relates to a genetically modified plant which has enhanced resistance to one or more fungal pathogen(s).

### BACKGROUND OF THE INVENTION

The partial stem rust resistance of Sr2 has remained effective for over a century in all wheat growing areas of the world including against the stem rust race Ug99 and its derivatives. Sr2 was introgressed into hexaploid wheat cv Marquis from the tetraploid emmer cultivar Yaroslav emmer (T. turgidum L.) in early 1900's (McFadden, 1930). The resulting variety, “Hope”, and its derivatives were used extensively as sources of stem rust resistance in North America, and in the Green Revolution semi-dwarf wheat varieties bred for use in South America, Asia and Africa (Rajaram et al., 1988). Sr2 is the most widely used race non-specific, Adult Plant Resistance (APR) gene catalogued as providing resistance to wheat stem rust (Puccinia graminis f. sp. tritici, Pgt; McIntosh et al., 1995; Singh et al., 2011). In addition, Sr2 is linked to resistance to leaf rust, Lr27 (Puccinia triticina), powdery mildew (Blumeria graminins f. sp. Tritici; Bgt) (Mago et al., 2011a) and stripe rust, Yr30 (Puccinia striiformis f. sp. tritici) resistance (Singh et al., 2001). Unlike Sr2, Lr27 confers race specific, all-stage resistance against leaf rust and requires a complementary gene Lr31 (Singh and McIntosh, 1984a, b) located on chromosome 4BL to confer resistance.

Expression of Sr2 stem rust resistance is often associated with a purple-brown to black coloration of stems and ears, termed pseudo black chaff (PBC). This phenotype occurs in the absence of pathogen inoculation and co-segregates perfectly with Sr2 resistance in mapping families (Kota et al., 2006; Mago et al., 2011b). However, expression of the PBC trait is modified by genetic background and environmental conditions, making it an imperfect visual marker for the presence of Sr2. PBC is associated with death and collapse of photosynthetic cells in the affected parts of stems and ears (Tabe et al., 2019). Abiotic stresses, such as heat shock and anoxia, also elicit leaf necrosis in Sr2 carrying genotypes (Tabe et al., 2019). The resistance response to fungal infection is also associated with death of photosynthetic cells around rust infection sites in inoculated leaf sheath in case of stem rust and death of leaf mesophyll cells around mildew infection sites (Tabe et al., 2019).

There is an ongoing need to expand resistance genetic resources, and to enhance gene stewardship through codeployment of multiple resistance genes to increase resistance durability.

### SUMMARY OF THE INVENTION

The present inventors have identified new polypeptides and genes which confer some level of resistance to plants against one or more fungal pathogen(s).

Thus, in a first aspect the present invention provides a plant having a genetic modification(s) and an increased level of
i) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
ii) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17,
   when compared to a corresponding wild-type plant lacking the genetic modification(s), wherein the plant has enhanced resistance to one or more fungal pathogen(s) when compared to the wild-type plant.

In another aspect, the present invention provides a plant having a genetic modification(s) and an increased level of
b) a polypeptide, wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
   ii) a polypeptide, wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17.

In an embodiment, the genetic modification(s) is an exogenous polynucleotide(s) encoding the polypeptide of part i) and/or ii).

In an embodiment, the polynucleotide(s) is operably linked to a promoter capable of directing expression of the polynucleotide(s) in a cell of the plant.

In an embodiment, the promoter directs gene expression in a leaf and/or stem cell.

In an embodiment, the one or more fungal pathogen(s) is a rust or a mildew or both a rust and a mildew. In an embodiment, the rust is stem rust or leaf rust. Examples of fungal pathogen(s) include, but are not limited to, is Puccinia sp., Blumeria sp., Fusarium sp., Magnoporthe sp., Bipolaris sp., Oidium sp., Gibberella sp., Cochliobolus sp., Exserohilum sp., Uredo sp. Microdochium sp., Helminthosporium sp., Monographella sp., Colletotrichum sp., Uromyces sp or Erysiphe sp.

In an embodiment, the polypeptide of part i) is encoded by a polynucleotide which comprises nucleotides having a sequence as provided in any one of SEQ ID NO's 18 to 26, a sequence which is at least 70% identical to one or more of SEQ ID NO's 18 to 26, or a sequence which hybridizes to one or more of SEQ ID NO's 18 to 26.

In an embodiment,
a) the polypeptide of part i) comprises amino acids having a sequence which is at least 90% identical to one or more of SEQ ID NO's 1 to 9, and/or
b) the polynucleotide of part i) comprises a sequence which is at least 90% identical to one or more of SEQ ID NO's 18 to 26.

In an embodiment,
a) the polypeptide of part i) comprises amino acids having a sequence which is at least 90% identical to SEQ ID NO:1 and/or SEQ ID NO:2, and/or
b) the polynucleotide of part i) comprises a sequence which is at least 90% identical to SEQ ID NO:18 or SEQ ID NO:19.

In an embodiment, the polypeptide of part ii) is encoded by a polynucleotide which comprises nucleotides having a sequence as provided in any one of SEQ ID NO's 27 to 34, a sequence which is at least 70% identical to one or more of SEQ ID NO's 27 to 34, or a sequence which hybridizes to one or more of SEQ ID NO's 27 to 34.

In an embodiment,
a) the polypeptide of part ii) comprises amino acids having a sequence which is at least 90% identical to any one of SEQ ID NO's 10 to 17, and/or
b) the polynucleotide of part ii) comprises a sequence which is at least 90% identical to any one of SEQ ID NO's 27 to 34.

In an embodiment,
a) the polypeptide of part ii) comprises amino acids having a sequence which is at least 90% identical to SEQ ID NO:10, and/or
b) the polynucleotide of part ii) comprises a sequence which is at least 90% identical to SEQ ID NO:27.

In an embodiment, the plant comprises
a) at least two polynucleotides encoding the polypeptide comprising amino acids having a sequence as provided in in any one of SEQ ID NO's 1 to 9, a biologically active fragment thereof, or an amino acid sequence which is at least 70% identical to one or more of SEQ ID NO's 1 to 9, and/or
b) at least two polynucleotides encoding the polypeptide comprising amino acids having a sequence as provided in any one of SEQ ID NO's 10 to 17, a biologically active fragment thereof, or an amino acid sequence which is at least 70% identical to one or more of SEQ ID NO's 10 to 17.

In an embodiment, the plant comprises
a) at least two polynucleotides encoding the polypeptide comprising amino acids having a sequence as provided in SEQ ID NO:1 or SEQ ID NO:2, a biologically active fragment thereof, or an amino acid sequence which is at least 70% identical to SEQ ID NO:1 and/or SEQ ID NO:2, and/or
b) at least two polynucleotides encoding the polypeptide comprising amino acids having a sequence as provided in SEQ ID NO:10, a biologically active fragment thereof, or an amino acid sequence which is at least 70% identical to SEQ ID NO:10.

In an embodiment, a plant of the invention has an increased copy number of the polynucleotide when compared to the corresponding wild-type plant.

In an embodiment, the plant comprises parts i) and ii). In an embodiment, the polynucleotides of i) and ii) are within 300 kb, 100 kb, 50 kb, or 20 to 30 kb of each other.

In an embodiment, the plant is a cereal plant. Examples of cereal plants of the invention include, but are not limited to, wheat, oats, rye, barley, rice, corn, sorghum or maize. In an embodiment, the plant is a wheat plant.

In an embodiment, the plant comprises one or more further genetic modifications encoding another plant pathogen resistance polypeptide. Examples of such other plant pathogen resistance polypeptides include, but are not limited to, Lr34, Lr1, Lr3, Lr2a, Lr3ka, Lr11, Lr13, Lr16, Lr17, Lr18, Lr21, LrB, Sr61, Lr67, Sr50, Sr33, Sr13, Sr26 and Sr35. In an embodiment, the plant further comprises Lr34 and Lr67.

In an embodiment, the plant is homozygous for one or more or all of the genetic modification(s).

In an embodiment, the plant is growing in a field.

Also provided is a population of at least 100 plants of the invention growing in a field.

In another aspect, the present invention provides a process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence as provided in any one of SEQ ID NO's 1 to 9, a biologically active fragment thereof, or an amino acid sequence which is at least 70% identical to one or more of SEQ ID NO's 1 to 9, ii) introducing the polynucleotide into a plant,
iii) determining whether the level of resistance to one or more fungal pathogen(s) is increased relative to a corresponding wild-type plant lacking the polynucleotide, and iv) optionally, selecting a polynucleotide which when expressed produces a polypeptide which confers enhanced resistance to one or more fungal pathogen(s).

In an embodiment,
a) the polypeptide comprises amino acids having a sequence which is at least 90% identical to one or more of SEQ ID NO's 1 to 9, and/or
b) the polynucleotide comprises a sequence which is at least 90% identical to one or more of SEQ ID NO's 18 to 26.

In an embodiment,
a) the polypeptide comprises amino acids having a sequence which is at least 90% identical to SEQ ID NO:1 and/or SEQ ID NO:2, and/or
b) the polynucleotide comprises a sequence which is at least 90% identical to SEQ ID NO:18 or SEQ ID NO:19.

In another aspect, the present invention provides a process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence as provided in any one of SEQ ID NO's 10 to 17 a biologically active fragment thereof, or an amino acid sequence which is at least 70% identical to one or more of SEQ ID NO's 10 to 17, ii) introducing the polynucleotide into a plant,
iii) determining whether the level of resistance to enhanced resistance to one or more fungal pathogen(s) is increased relative to a corresponding wild-type plant lacking the polynucleotide, and
iv) optionally, selecting a polynucleotide which when expressed produces a polypeptide which confers enhanced resistance to one or more fungal pathogen(s).

In an embodiment,
a) the polypeptide comprises amino acids having a sequence which is at least 90% identical to any one of SEQ ID NO's 10 to 17, and/or
b) the polynucleotide comprises a sequence which is at least 90% identical to any one of SEQ ID NO's 27 to 34.

In an embodiment,
a) the polypeptide comprises amino acids having a sequence which is at least 90% identical to SEQ ID NO:10, and/or
b) the polynucleotide comprises a sequence which is at least 90% identical to SEQ ID NO:27.

In an embodiment, the plant is a cereal plant such as a wheat plant.

In an embodiment, step ii) of the above two aspects further comprises stably integrating the polynucleotide operably linked to a promoter into the genome of the plant.

In an embodiment, the plant of step iii) of the above two aspects comprises a first polynucleotide encoding a polypeptide comprising amino acids having a sequence as provided in any one of SEQ ID NO's 1 to 9, a biologically active fragment thereof, or an amino acid sequence which is at least 70% identical to one or more of SEQ ID NO's 1 to 9, and a second polynucleotide encoding a polypeptide comprising amino acids having a sequence as provided in any one of SEQ ID NO's 10 to 17 a biologically active fragment thereof, or an amino acid sequence which is at least 70% identical to one or more of SEQ ID NO's 10 to 17.

In another aspect the present invention further provides a substantially purified and/or recombinant polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9.

In an embodiment, the polypeptide comprises amino acids having a sequence which is at least 70% identical, at least 80% identical, at least 90% identical, or at least 95% identical, to SEQ ID NO:1 and/or SEQ ID NO:2.

In another aspect the present invention further provides a substantially purified and/or recombinant polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17.

In an embodiment, the polypeptide comprises amino acids having a sequence which is at least 70% identical, at least 80% identical, at least 90% identical, or at least 95% identical, to SEQ ID NO:10.

In an embodiment of the two above aspects the polypeptide is a fusion protein further comprising at least one other polypeptide sequence. The at least one other polypeptide may be, for example, a polypeptide that enhances the stability of a polypeptide of the present invention, or a polypeptide that assists in the purification or detection of the fusion protein.

In another aspect the present invention further provides an isolated and/or exogenous polynucleotide comprising nucleotides having a sequence as provided in any one of SEQ ID NO's 18 to 26, a sequence which is at least 70% identical to one or more of SEQ ID NO's 18 to 26, a sequence encoding a polypeptide of the invention, or a sequence which hybridizes to one or more of SEQ ID NO's 18 to 26.

In another aspect the present invention further provides an isolated and/or exogenous polynucleotide comprising nucleotides having a sequence as provided in any one of SEQ ID NO's 27 to 34, a sequence which is at least 70% identical to one or more of SEQ ID NO's 27 to 34, a sequence encoding a polypeptide of the invention, or a sequence which hybridizes to one or more of SEQ ID NO's 27 to 34.

Also provided is a chimeric vector comprising the polynucleotide of the invention and/or the polynucleotide of the invention.

In an embodiment, the polynucleotide is operably linked to a promoter.

In an embodiment, the vector comprises one or more further exogenous polynucleotides encoding another plant pathogen resistance polypeptide as described herein.

In another aspect, the present invention provides a recombinant cell comprising an exogenous polynucleotide of the invention, and/or a vector of the invention.

The cell can be any cell type such as, but not limited to, a plant cell, a bacterial cell, an animal cell or a yeast cell.

Preferably, the cell is a plant cell. More preferably, the plant cell is a cereal plant cell. Even more preferably, the cereal plant cell is a wheat cell.

In another aspect, the present invention provides a method of producing a polypeptide of the invention, the method comprising expressing in a cell or cell free expression system a polynucleotide of the invention.

Preferably, the method further comprises isolating the polypeptide.

In another aspect, the present invention provides a transgenic non-human organism, such as a transgenic plant, comprising an exogenous polynucleotide of the invention, a vector of the invention and/or a recombinant cell of the invention.

In another aspect, the present invention provides a method of producing the cell of the invention, the method comprising the step of introducing a polynucleotide of the invention, or a vector of the invention, into a cell.

In another aspect, the present invention provides a method of producing a plant with a genetic modification(s) of the invention, the method comprising the steps of
i) introducing a genetic modification(s) to a plant cell which increases the expression level of
   a) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
   b) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17,
      when compared to a corresponding wild-type plant cell lacking the genetic modification(s),
ii) regenerating a plant with the genetic modification(s) from the cell, and
iii) optionally harvesting seed from the plant, and/or
iv) optionally producing one or more progeny plants from the genetically modified plants,
   thereby producing the plant.

In an embodiment, step i) comprises introducing a polynucleotide of the invention and/or a vector of the invention into the plant cell.

In another aspect, the present invention provides a method of producing a plant with a genetic modification(s) of the invention, the method comprising the steps of
i) crossing two parental plants, wherein at least one plant comprises a genetic modification(s) of the invention,
ii) screening one or more progeny plants from the cross in i) for the presence or absence of the genetic modification(s), and
iii) selecting a progeny plant which comprise the genetic modification (s), thereby producing the plant.

In an embodiment, at least one of the parental plants is a tetraploid or hexaploid wheat plant.

In an embodiment, step ii) comprises analysing a sample comprising DNA from the plant for the genetic modification(s).

In an embodiment, step iii) comprises
i) selecting progeny plants which are homozygous for the genetic modification(s), and/or
ii) analysing the plant or one or more progeny plants thereof for enhanced resistance to one or more fungal pathogen(s).

In an embodiment, the method further comprises
iv) backcrossing the progeny of the cross of step i) with plants of the same genotype as a first parent plant which lacked the genetic modification(s) for a sufficient number of times to produce a plant with a majority of the genotype of the first parent but comprising the genetic modification(s), and
v) selecting a progeny plant which has enhanced resistance to one or more fungal pathogen(s).

Also provided is a plant produced using a method of the invention.

Further provided is the use of polynucleotide of the invention, or a vector of the invention, to produce a recombinant cell and/or a transgenic plant. In another embodiment, the use of the polynucleotide of the invention is with an enzyme having endonuclease activity to increase the level of the polypeptide.

In an embodiment, the transgenic plant has enhanced resistance to one or more fungal pathogen(s) when compared to a corresponding wild-type plant lacking the exogenous polynucleotide and/or vector.

In another aspect, the present invention provides a method for identifying a plant which has enhanced resistance to one or more fungal pathogen(s), the method comprising the steps of
i) obtaining a sample from a plant, and
ii) a) screening the sample for the presence or absence of a genetic modification(s) which increases the level of
   I) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
   II) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17, and/or
b) screening the sample for the level of the polypeptide defined in I) and/or II).

In an embodiment, the screening comprises amplifying a region of the genome of the plant. In an embodiment, the amplification is achieved using an oligonucleotide primer comprising a sequence of nucleotides provided as any one of SEQ ID NO's 43 to 55 and 62 to 68, or a variant thereof which can be used to amplify the same region of the genome. In an embodiment, the primer comprises a sequence of nucleotides provided as any one of SEQ ID NO's 46 to 49, or a variant thereof which can be used to amplify the same region of the genome.

In an embodiment, the method identifies a genetically modified plant of the invention.

Also provided is a plant part of the plant of the invention. In an embodiment, the plant part is a seed that comprises the genetic modification(s).

In another aspect, the present invention provides a method of producing a plant part, the method comprising,
a) growing a plant of the invention, and
b) harvesting the plant part.

In another aspect, the present invention provides a method of producing flour, wholemeal, starch or other product obtained from seed, the method comprising;
a) obtaining seed of the invention, and
b) extracting the flour, wholemeal, starch or other product.

In another aspect, the present invention provides a product produced from a plant of the invention and/or a plant part of the invention.

In an embodiment, the part is a seed.

In an embodiment, the product is a food product or beverage product. Examples of food products include, but are not limited to, flour, starch, leavened or unleavened breads, pasta, noodles, animal fodder, animal feed, breakfast cereals, snack foods, cakes, malt, beer, pastries and foods containing flour-based sauces. Examples of beverage products include, but are not limited to, beer or malt.

In an embodiment, the product is a non-food product. Examples include, but are not limited to, films, coatings, adhesives, building materials and packaging materials.

In another aspect, the present invention provides a method of preparing a food product of the invention, the method comprising mixing seed, or flour, wholemeal or starch from the seed, with another food ingredient.

In another aspect, the present invention provides a method of preparing malt, comprising the step of germinating seed of the invention.

Also provided is the use of a plant of the invention, or part thereof, as animal feed, or to produce feed for animal consumption or food for human consumption.

In another aspect, the present invention provides a method of producing flour, the method comprising;
i) obtaining cereal grain,
ii) grinding the grain,
iii) sifting the ground grain, and
iv) recovering the flour,
   wherein the cereal grain has a genetically modified gene encoding a PMP3 and/or PMP4 polypeptide.

In a further aspect, the present invention provides a method of producing malt, the method comprising;
i) obtaining cereal grain,
ii) steeping the grain,
iii) germinating the steeped grains,
iv) drying the germinated grain, and
v) recovering the malt,
   wherein the cereal grain has a genetically modified gene an PMP3 and/or PMP4 polypeptide.

In a further aspect the present invention provides of the use of a plant of the invention for controlling or limiting one or more fungal pathogen(s) in crop production.

In another aspect, the present invention provides a composition comprising one or more of a polypeptide of the invention, a polynucleotide of the invention, a vector of the invention, or a recombinant cell of the invention, and one or more acceptable carriers.

In another aspect, the present invention provides a method of trading seed, comprising obtaining seed of the invention, and trading the obtained seed for pecuniary gain.

In an embodiment, obtaining the seed comprises cultivating the plant of the invention, and/or harvesting the seed from the plants.

In an embodiment, obtaining the seed further comprises placing the seed in a container and/or storing the seed.

In an embodiment, obtaining the seed further comprises transporting the seed to a different location.

In an embodiment, the trading is conducted using electronic means such as a computer.

In another aspect, the present invention provides a process of producing bins of seed comprising:
a) swathing, windrowing and/or or reaping above-ground parts of plants comprising seed of the invention,
b) threshing and/or winnowing the parts of the plants to separate the seed from the remainder of the plant parts, and
c) sifting and/or sorting the seed separated in step b), and loading the sifted and/or sorted seed into bins, thereby producing bins of seed.

In another aspect, the present invention provides a method of identifying a compound that binds to a polypeptide comprising amino acids having a sequence as provided in any one of SEQ ID NO's 1 to 17, a biologically active fragment thereof, or an amino acid sequence which is at least 70% identical to any one or more of SEQ ID NO's 1 to 17, the method comprising:
i) contacting the polypeptide with a candidate compound, and
ii) determining whether the compound binds the polypeptide.

Also provided is the use of an isolated and/or exogenous polynucleotide for the production of a genetically modified plant, wherein when present in the plant increases the expression of a gene encoding
i) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
ii) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—(A) Genotypes of the recombinants at the Sr2 locus in the Marquis×CS(Hope3B) mapping F2 family. ‘A’ represents the susceptible parent allele, resistant parent allele is represented by ‘B’. The plants were phenotyped in the field in Canberra, Australia and Minnesota, USA. The phenotypes are represented as R=resistant, MR=moderately resistant, MS=moderately susceptible, MRMS=moderately resistant to moderately susceptible, MSS=moderately susceptible to susceptible, S=susceptible, SHS=susceptible to highly susceptible. The phenotyping in the USA also included rust severity scores (0-100). Phenotyping of recombinant MC22 (AB) indicates that the resistance is located distal to TaGLP3-8/9. **marker Dox-1 which defines the distal end of the locus in recombinant R7.1, was not polymorphic in this cross but was mapped previously in CS×CS(Hope3B) cross and thus not checked in recombinants and represented as NA. (B) Stem rust infection phenotypes at 15 dpi. (C) PBC phenotype of Marquis×CS-Hope3B recombinant MC22 and the parents. For rust phenotyping plants were inoculated at anthesis with Pgt race 21-0 and scored at 11, 13, 15- and 17-days post inoculation to derive the S or R phenotype score shown below each line.

FIG. 2—(A) Physical map of the Sr2 locus showing the position of markers used for screening of recombinants in the Marquis×CS-Hope3B mapping family. Genes highlighted re recombining. (B) Revised physical map of the Sr2 locus based on long-read sequencing showing position of annotated genes between the recombing markers CSSr2 and DOX_1. (C) A detailed schematic diagram of the duplicated region showing the six annotated genes in the region represented in different colors, TPR1, D8LAL2-TaPMP-B4, Unknown, CA687088, CD882879-TaPMPB3a and TPR2. The duplication junctions (left, middle, right) are also indicated and the unique primer combination that identifies the duplication is indicated at the bottom. (D) Self dot plot analysis of the genomic sequence of the Sr2 locus showing duplications at the locus after resequencing using the long-read Nanopore sequencing. Dots correspond to sense duplications and anti sense duplications. (E) shows PCR amplification of markers across duplication junctions, both left and right junctions are present in all the samples, while the middle junction amplifies only in Sr2 wheats confirming that the duplication is unique to Sr2 positive lines. Lanes, M, size marker, 1. CS, 2. CS(Hope3B) [CSH], 3. Yaroslav emmer (Sr2 donor), 4. Hope (original Sr2 carrying wheat), 5. Marquis (susceptible parent), 6. wheat cv. Mace. (F) Relative copy number estimation of TaPMP-B3 using digital droplet PCR in CS(Hope3B) compared to Marquis (set to 1).

FIG. 3—Copy number analysis across part of Sr2 locus carrying the candidate genes and duplicated region (indicated). (A) Absolute read depth across the Sr2 locus genome region for CS(Hope3B) (Blue) and Marquis (red) plotted in 100 bp bins. (B) Relative read depth across the region plotted as the ratio of read depth in CS(Hope3B) versus Marquis in 10-0 bp bins.

FIG. 4—Gene expression analysis (RNAseq) of genes at Sr2 locus (A) expression of genes at Sr2 locus is represented as Reads per kilobase per million (RPKM) in stem rust infected leaves in CS(Hope3B) [CSH] at 0 and 48 hrs post infection with Pgt 98-1,2,3,5,6. Genes CD882879 (TaPMP-B3) and D8LAL2 (TaPMP-B4) are highly expressed upon pathogen infection compared to other genes at the locus. (B-D) Expression analysis by qRT-PCR of D8LAL2 and CD882879 at 0 and 48 hrs post infection with Pgt 21-0 in CS(Hope3B) and Marquis relative to TaCon (control). Gene expression in (B) stem rust-inoculated sheath of adult plants at anthesis, (C) leaves of powdery mildew infected 5-week-old plants, and (D) stem rust (Pgt 21-0) infected 1-week old seedlings at 0 and 48 hrs post infection. (E) Expression of all the genes annotated on the 90 kb duplicated region in plants infected with stem rust inoculated flag-1 leaf at anthesis. Samples were collected at 0 and 48 hours post infection.

FIG. 5—Sequence analysis of Sr2 candidate gene sequences (A) Amino acid sequence comparison of CD882879 (TaPMP-B3) between the resistant [CS(Hope3B-TaPMP-B3a] and susceptible (Marquis-TaPMP-B3b) showing a single amino acid difference. (B) Prediction of transmembrane helices in proteins TaPMP-B3. (C) TaPMP-B4 (D8LAL2). TaPMP-B3 encodes a 374 amino acid protein with 10 predicted transmembrane domains while TaPMP-B4 encodes a 406 amino acid protein with 7 predicted transmembrane domains. (D) Structures of TaPMP-B3a and TaPMP-B4 proteins as predicted by AlphaFold V2.1.1. (E) Map showing schematic organization of genes TaPMP-B3 and TaPMP-B4 on chromosome 3B on CS(Hope3B), the genes are separated by 22.5 kb intervening sequence.

FIG. 6—Map of hairpin RNAi silencing vector pSTARGATE (Greenup et al., 2010) showing the position of the sense and antisense fragments of candidate gene TaPMP-B3a.

FIG. 7—Analysis of T1 progeny of TaPMP-B3a-RNAi transgenics from 2 independent events TE1 and TE2. Individual plants within a family are indicated. Presence of transgene is shown by ‘+’ or ‘−’. (A) Northern blot analysis on transgenic plants showing gene expression. Plants carrying the transgene show suppression of TaPMP-B3 expression compared to WT and null segregants. (B) shows loading standard of above samples represented by rRNA (C) Stem rust infection phenotypes of transgenic plants in TE1 and TE2. Plants were infected with Pgt race 98-1,2,3,5,6 at anthesis and scored at 11 DPI. Rust phenotypes are indicated at the bottom. Plants carrying the transgene were scored as MSS to SHS while plants lacking the transgene behaved similar to the resistant parent.

FIG. 8—Biotic and abiotic phenotypes of TaPMP-B3a (TaPMP-B3a-hp) silenced plants of transgenic events TE1, TE2 and T3. Presence of transgene is indicated by ‘+’ or ‘−’ (A) Leaf rust phenotype of the segregating T1 progeny infected with Pt race 122-1,2,3,5 (PBI #351). CS (Chinese Spring) is susceptible compared to CS(Hope3B) (CSH) which is resistant. Plants carrying transgene are susceptible. (B) Powdery mildew (PM) phenotypes of segregating T2 progeny from TE1 and TE2. Plants carrying transgene are susceptible while the null segregants and the CSH control are resistant. (C) T1 sib lines showing loss of Vaseline induced necrosis in transgenic plants while the null shows necrosis, characteristic for presence of Sr2 resistance. (D) Heat shock induced leaf necrosis in PMP3-silenced T2 progeny from TE1 and TE2. Transgenic plants lack the necrotic phenotype. (E) Whole plant phenotype of transgenic vs a null segregant at T1 generation.

FIG. 9—Map of vector containing the Sr2 candidate gene, TaPMP-B3a expressed under Maize Ubiquitin protomer used for Agrobacterium transformation of wheat cv Marquis.

FIG. 10—(A) Stem rust phenotypes of T2 segregating lines of Marquis transgenics carrying TaPMP-B3a from two independent events TE1 and TE2. Lines 3, 6 and 14-1 are T2 progeny from TE1 while lines 15, 17, 19 and 22 are derived from TE2. ‘+’ indicates presence of transgene while ‘-’ lines are nulls. Plants were infected at anthesis with Pgt race 21-0 and scored at 9 DPI. (B) Shows Vaseline induced necrosis in segregating lines from TE1. (C) In planta quantification of fungal biomass (relative fluorescence units, RFU) on of T2 segregating lines of Marquis transgenics carrying the Sr2 candidate gene PMP3 from two independent events TE1, TE2 and resistant and susceptible parents by wheat germ agglutinin chitin (WAC) assay. Infected sheath were collected from plants 9 DPI, weighed and freeze dried before grinding. Four technical replicates were produced for each sample and shown as average with error bars. Individual values are seen as Black dots. (D) Relative gene expression of PMP3 in T2 lines that are segregating for transgene and the resistant parent CSH and susceptible Marquis at 0 hrs and 48 hrs post rust infection.

FIG. 11—(A) Map of hairpin RNAi silencing vector pSTARGATE (Greenup et al., 2010) showing the position of the sense and antisense fragments of candidate gene TaPMP-B4. (B) Map of vector containing the Sr2 candidate gene, TaPMP-B4 expressed under Maize Ubiquitin protomer used for Agrobacterium transformation of wheat cv Marquis (C) Necrotic phenotypes of the T2 transgenic lines carrying the TaPMP-B4 hairpin construct. The phenotype was scored on a scale 0-4 with ‘0’ being no necrosis and ‘4’ showing extreme. Vaseline was applied to leaves of 5-week-old plants and scored for necrosis 1 week after application. The area where Vaseline was applied is shown by blue dotted area. T2 plant numbers are shown at the bottom and correlate to plant number in Table 6. Presence or absence of transgene (Hairpin) is also indicated. (D) Stem rust phenotypes of silenced plants of a single transgenic event at 11 DPI. (E) Leaf rust phenotype and (F) Powdery mildew phenotype of silenced plants from 2 transgenic events. Plants carrying the transgene (silenced) are susceptible to the corresponding pathogen. CS and CSH were included as susceptible and resistant controls.

FIG. 12—(A) In planta cell death scores in leaf tissue expressing TaPMP-B3 and TaPMP-B4 variants alone and in combination. YFP and Sr50CC-YFP were used as negative and positive controls in this experiment, respectively. The severity of cell death phenotypes were scored on a scale of 0 to 5 as shown and the graph shows scores for individual infiltrations as dots with at least 8 replicates for each treatment. (B). Representative N. benthamiana leaf photos showing the results described in FIG. 11A.

FIG. 13—(A) Comparison of cell death induced by co-expression of TaPMP-B3a or TaPMP-B3b with TaPMP-B4 in N. benthiamiana or N. tabacum with or without LaCl3 treatment. (B) Protein structure prediction of TaPMP-3Ba and TaPMP-B4 together using AlphaFold v2.1.1 indicating the potential ion channel formed in the dimer.

FIG. 14—Phylogenetic tree of orthologous protein sequences for TaPMP3a and TaPMP4 located on homologous group 3 in wheat (Triticum aestivum), and syntenic chromosomal positions on 3H in barley (Hordeum vulgare), chromosome 1 in rice (Oryza sativa), unanchored scaffold in tef (Eragrostis tef), chromosome 3 in maize (Zea mays) and chromosome 3 in sorghum (Sorghum bicolor).

FIG. 15—Predicted structures of TaPMP-B3a homologs from plant species listed in Table 8.

FIG. 16—Predicted structures of TaPMP-B3a homologs from plant species listed in Table 8.

### KEY TO THE SEQUENCE LISTING

SEQ ID NO:1—Amino acid sequence of wheat TaPMP-B3a polypeptide.
SEQ ID NO:2—Amino acid sequence of wheat TaPMP-B3b polypeptide.
SEQ ID NO:3—Amino acid sequence of wheat TaPMP-B3 polypeptide ortholog encoded by 3A genome.
SEQ ID NO:4—Amino acid sequence of wheat TaPMP-B3 polypeptide ortholog encoded by 3D genome.
SEQ ID NO:5—Amino acid sequence of barley TaPMP-B3 polypeptide ortholog.
SEQ ID NO:6—Amino acid sequence of rice TaPMP-B3 polypeptide ortholog.
SEQ ID NO:7—Amino acid sequence of maize TaPMP-B3 polypeptide ortholog.
SEQ ID NO:8—Amino acid sequence of sorghum TaPMP-B3 polypeptide ortholog.
SEQ ID NO:9—Amino acid sequence of tef TaPMP-B3 polypeptide ortholog.
SEQ ID NO:10—Amino acid sequence of wheat TaPMP-B4a polypeptide.
SEQ ID NO:11—Amino acid sequence of wheat TaPMP-B4 polypeptide ortholog encoded by 3A genome.
SEQ ID NO:12—Amino acid sequence of wheat TaPMP-B4 polypeptide ortholog encoded by 3D genome.
SEQ ID NO:13—Amino acid sequence of barley TaPMP-B4 polypeptide ortholog.
SEQ ID NO:14—Amino acid sequence of rice TaPMP-B4 polypeptide ortholog.
SEQ ID NO:15—Amino acid sequence of maize TaPMP-B4 polypeptide ortholog.
SEQ ID NO:16—Amino acid sequence of sorghum TaPMP-B4 polypeptide ortholog.
SEQ ID NO:17—Amino acid sequence of tef TaPMP-B4 polypeptide ortholog.
SEQ ID NO:18—Open reading frame encoding wheat TaPMP-B3a polypeptide.
SEQ ID NO:19—Open reading frame encoding wheat TaPMP-B3b polypeptide.
SEQ ID NO:20—Open reading frame encoding wheat TaPMP-B3 polypeptide ortholog encoded by 3A genome.
SEQ ID NO:21—Open reading frame encoding wheat TaPMP-B3 polypeptide ortholog encoded by 3D genome.
SEQ ID NO:22—Open reading frame encoding barley TaPMP-B3 polypeptide ortholog.
SEQ ID NO:23—Open reading frame encoding rice TaPMP-B3 polypeptide ortholog.
SEQ ID NO:24—Open reading frame encoding maize TaPMP-B3 polypeptide ortholog.
SEQ ID NO:25—Open reading frame encoding sorghum TaPMP-B3 polypeptide ortholog.
SEQ ID NO:26—Open reading frame encoding tef TaPMP-B3 polypeptide ortholog.
SEQ ID NO:27—Open reading frame encoding wheat TaPMP-B4a polypeptide.
SEQ ID NO:28—Open reading frame encoding wheat TaPMP-B4 polypeptide ortholog encoded by 3A genome.
SEQ ID NO:29—Open reading frame encoding wheat TaPMP-B4 polypeptide ortholog encoded by 3D genome.
SEQ ID NO:30—Open reading frame encoding barley TaPMP-B4 polypeptide ortholog.
SEQ ID NO:31—Open reading frame encoding rice TaPMP-B4 polypeptide ortholog.
SEQ ID NO:32—Open reading frame encoding maize TaPMP-B4 polypeptide ortholog.
SEQ ID NO:33—Open reading frame encoding sorghum TaPMP-B4 polypeptide ortholog.
SEQ ID NO:34—Open reading frame encoding tef TaPMP-B4 polypeptide ortholog.
SEQ ID NO:35—Genomic region encoding wheat TaPMP-B3a polypeptide.
SEQ ID NO:36—Genomic region encoding wheat TaPMP-B3b polypeptide.
SEQ ID NO:37—Genomic region encoding wheat TaPMP-B4a polypeptide.
SEQ ID NO's 38 to 70 and 72 to 84—Oligonucleotide primers.
SEQ ID NO: 71-90 kb region of Triticum aestivum cv Hope comprising TaPMP-B3 and TaPMP-B4.
SEQ ID NO:85—Amino acid sequence of cultivar Chinese Spring (wheat) TaPMP-B3a polypeptide.
SEQ ID NO:86—Amino acid sequence of cultivar Chinese Spring (wheat) TaPMP-B4 polypeptide.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, rust resistance, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, of the particular term.

### Polypeptides

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 90% free from other components with which it is naturally associated.

Genetically modified plants and host cells, such as transgenic plants or cells, of the invention may comprise an exogenous polynucleotide encoding a polypeptide of the invention. In these instances, the plants and cells produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation or gene editing. Typically, the cell comprises a non-endogenous gene that causes an altered amount of the polypeptide to be produced. In an embodiment, the endogenous gene has been modified to increase its expression, such as by replacing the native promoter with one that results in increased gene expression. In an embodiment, a “recombinant polypeptide” is a polypeptide made by the expression of an exogenous (recombinant) polynucleotide in a plant cell.

As used herein, an “increased level” of one or more polypeptides defined herein when compared to a corresponding wild-type plant lacking the genetic modification(s) means that a genetically identical plant lacking the genetic modification(s) produces less of the one or more polypeptides defined herein than a genetically modified plant of the invention. In an embodiment, the genetically modified plant of the invention produces at least twice as much of the one or more polypeptides defined herein when compared to a corresponding wild-type plant, for example twice as much in the stems of the plant. In an embodiment, the genetically modified plant of the invention has an endogenous gene(s) which encodes one or more polypeptides defined herein, and one or more exogenous polynucleotides encoding one or more polypeptides defined herein.

The terms “polypeptide” and “protein” are generally used interchangeably.

In an embodiment, a polypeptide of the invention which confers enhanced resistance to one or more fungal pathogen(s), and which comprises amino acids having a sequence as provided in any one of SEQ ID NO's 1 to 9, a biologically active fragment thereof, or an amino acid sequence which is at least 70% identical to one or more of SEQ ID NO's 1 to 9, has 8 to 11, or 9 or 10, or 10 transmembrane domains. I

In an embodiment, a polypeptide of the invention which confers enhanced resistance to one or more fungal pathogen(s), and which comprises amino acids having a sequence as provided in any one of SEQ ID NO's 10 to 17 a biologically active fragment thereof, or an amino acid sequence which is at least 70% identical to one or more of SEQ ID NO's 10 to 17, has 6 to 10, or 6 or 7, or 7 transmembrane domains.

As used herein, the term “PMP3 polypeptide” relates to a protein family which share high primary amino acid sequence identity, for example, at least 70%, least 80%, at least 90%, or at least 95% identity with amino acids having a sequence as provided in any one of SEQ ID NO's 1 to 9. In embodiment, the PMP3 polypeptide has at least 70%, least 80%, at least 90%, or at least 95% identity with amino acids having a sequence as provided in any one of SEQ ID NO's 1 to 9 or 85. The present inventors have determined that this protein family, when expressed in high enough levels in a plant, confer upon the plant resistance to at least one or more fungal pathogens.

As used herein, the term “PMP4 polypeptide” relates to a protein family which share high primary amino acid sequence identity, for example, at least 70%, least 80%, at least 90%, or at least 95% identity with amino acids having a sequence as provided in any one of SEQ ID NO's 10 to 17. In embodiment, the PMP3 polypeptide has at least 70%, least 80%, at least 90%, or at least 95% identity with amino acids having a sequence as provided in any one of SEQ ID NO's 10 to 17 or 86. The present inventors have determined that this protein family, when expressed in high enough levels in a plant, confer upon the plant resistance to at least one or more fungal pathogens.

The % identity of a polypeptide is determined by GAP (Needleman and Wunsch, 1970) analysis (GCG program) with a gap creation penalty=5, and a gap extension penalty=0.3. The query sequence is at least 300 amino acids in length, and the GAP analysis aligns the two sequences over a region of at least 300 amino acids. More preferably, the query sequence is at least 350 amino acids in length and the GAP analysis aligns the two sequences over a region of at least 350 amino acids. Even more preferably, the query sequence is at least 374 amino acids in length and the GAP analysis aligns the two sequences over a region of at least 374 amino acids. Even more preferably, the GAP analysis aligns two sequences over their entire length.

As used herein a “biologically active” fragment is a portion of a polypeptide of the invention which maintains a defined activity of the full-length polypeptide such as when expressed in a plant, such as wheat, confers (enhanced) resistance to one or more fungal pathogen(s) when compared to a corresponding wild-type plant not expressing the polypeptide. Biologically active fragments can be any size as long as they maintain the defined activity but are preferably at least 350 or at least 374 amino acid residues long. Preferably, the biologically active fragment maintains at least 10%, at least 50%, at least 75% or at least 90%, of the activity of the full length protein. In an embodiment, the biologically active fragment comprises the same number of transmembrance domain as the corresponding full length protein.

In an embodiment, PMP43 polypeptide and PMP4 polypeptide form a dimer. In an embodiment, PMP43 polypeptide and PMP4 polypeptide form an ion channel. In an embodiment, PMP43 polypeptide and PMP4 polypeptide form a calcium ion channel.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In an embodiment, a polypeptide of the invention is not a naturally occurring polypeptide.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have one, two, three, four or less than 10 amino acid changes relative to the reference wildtype polypeptide.

Mutant (altered) polypeptides can be prepared using any technique known in the art, for example, using CRISPR Cas 9 or alternative endonucleases, directed evolution, rational design strategies or mutagenesis (see below). Products derived from mutated/altered DNA can readily be screened using techniques described herein to determine if, when expressed in a plant, such as wheat, confer (enhanced) resistance to one or more fungal pathogen(s). For instance, the method may comprise producing a transgenic plant expressing the mutated/altered DNA and determining the effect of the pathogen on the growth of the plant.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 1 under the heading of “exemplary substitutions”.

In a preferred embodiment a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different polypeptides provided herewith. As the skilled person would be aware, such minor changes can reasonably be predicted not to alter the activity of the polypeptide when expressed in a recombinant cell.

The primary amino acid sequence of a polypeptide of the invention can be used to design variants/mutants thereof based on comparisons with closely related polypeptides, such as aligning two or more of the amino acid sequences provided as SEQ ID NO's 1 to 9, or aligning two or more of the amino acid sequences provided as SEQ ID NO's 10 to 17. As the skilled addressee will appreciate, residues highly conserved amongst closely related proteins are less likely to be able to be altered, especially with non-conservative substitutions, and activity maintained than less conserved residues (see above).

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In an embodiment, a PMP3 polypeptide of the invention comprises an arginine, or a conservative substitution thereof, at an amino acid position corresponding to amino acid number 9 of SEQ ID NO:85. In an embodiment, a PMP3 polypeptide of the invention comprises a glutamic acid, or a conservative substitution thereof, at an amino acid position corresponding to amino acid number 13 of SEQ TD NO:85. In an embodiment, a PMP3 polypeptide of the invention comprises a glutamine, or a conservative substitution thereof, at an amino acid position corresponding to amino acid number 68 of SEQ ID NO:85. In an embodiment, a PMP3 polypeptide of the invention comprises a threonine, or a conservative substitution thereof, at an amino acid position corresponding to amino acid number 117 of SEQ ID NO:85. In an embodiment, a PMP3 polypeptide of the invention comprises a glutamic acid, or a conservative substitution thereof, at an amino acid position corresponding to amino acid number 269 of SEQ ID NO:85. In an embodiment, a PMP4 polypeptide of the invention comprises a glutamic acid, or a conservative substitution thereof, at an amino acid position corresponding to amino acid number 204 of SEQ ID NO:86. In an embodiment, a PMP4 polypeptide of the invention comprises a lysine, or a conservative substitution thereof, at an amino acid position corresponding to amino acid number 208 of SEQ ID NO:86. In an embodiment, a PMP4 polypeptide of the invention comprises a arginine, or a conservative substitution thereof, at an amino acid position corresponding to amino acid number 195 of SEQ ID NO:86. In an embodiment, a PMP4 polypeptide of the invention comprises a glutamic acid, or a conservative substitution thereof, at an amino acid position corresponding to amino acid number 251 of SEQ ID NO:86. In an embodiment, a PMP4 polypeptide of the invention comprises a tryptophan, or a conservative substitution thereof, at an amino acid position corresponding to amino acid number 3 of SEQ ID NO:10.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. The polypeptides may be post-translationally modified in a cell, for example by phosphorylation, which may modulate its activity. These modifications may serve to increase the stability and/or bioactivity of the polypeptide of the invention.

### Directed Evolution

In directed evolution, random mutagenesis is applied to a protein, and a selection regime is used to pick out variants that have the desired qualities, for example, increased activity. Further rounds of mutation and selection are then applied. A typical directed evolution strategy involves three steps:
1) Diversification: The gene encoding the protein of interest is mutated and/or recombined at random to create a large library of gene variants. Variant gene libraries can be constructed through error prone PCR (see, for example, Leung. 1989; Cadwell and Joyce, 1992), from pools of DNaseI digested fragments prepared from parental templates (Stemmer, 1994a; Stemmer, 1994b; Crameri et al., 1998; Coco et al., 2001) from degenerate oligonucleotides (Ness et al., 2002, Coco, 2002) or from mixtures of both, or even from undigested parental templates (Zhao et al., 1998; Eggert et al., 2005; Jézéquek et al., 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).
2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a host organism or part thereof and assaying the level of activity.
3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interative Protein Redesign and Optimization), EGAD (A Genetic Algorithm for Protein Design), Rosetta Design, Sharpen and Abalone.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. Less preferred polynucleotides include tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. A “hybridized polynucleotide” means the polynucleotide is actually basepaired to its complement. The term “polynucleotide” is used interchangeably herein with the term “nucleic acid”. Preferred polynucleotides of the invention encode a polypeptide of the invention.

By “isolated polynucleotide” we mean a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

The present invention involves modification of gene activity which may involve the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region of a polynucleotide of the invention is operably linked to a promoter or polyadenylation/terminator region which is heterologous to the native gene, thereby forming a chimeric gene. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous polynucleotides may be the result of editing the genome of the organism or cell, or progeny derived therefrom. A “transgene” is a gene that has been introduced into the genome by a transformation procedure. The term “genetically modified” includes introducing genes into cells by transformation or transduction, gene editing, mutating genes in cells and altering or modulating the regulation of a gene in a cell or organisms to which these acts have been done or their progeny.

Furthermore, the term “exogenous” in the context of a polynucleotide (nucleic acid) refers to a polynucleotide whose presence in a cell has come from external means, it can include introducing a polynucleotide in a cell that does not naturally comprise the polynucleotide or increasing the copy number of a polynucleotide within a cell by introducing one or more copies of the polynucleotide into a cell comprising an endogenous polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 1,050 nucleotides in length, and the GAP analysis aligns the two sequences over a region of at least 1,050 nucleotides. Even more preferably, the query sequence is at least 1,122 nucleotides in length and the GAP analysis aligns the two sequences over a region of at least 1,122 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for a genome editing enzyme enzymes (e.g. an endonuclease such as CRISPR Cas9), probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule. Examples of oligonucleotides of the invention include those with a nucleotide sequence provided as any one of SEQ ID NO's 38 to 68 and 72 to 84, in particular SEQ ID NO's 43 to 55 and 62 to 68.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences provided as SEQ ID NO's 18 to 34, 35 to 37 or 71, preferably any one or more of SEQ ID NOs 18, 19, 27, 35 to 37 or 71. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis or genome editing on the nucleic acid). A variant of a polynucleotide or an oligonucleotide of the invention includes molecules of varying sizes of, and/or are capable of hybridising to, the wheat genome close to that of the reference polynucleotide or oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close to, for example to within 50 nucleotides, the region of the plant genome where the specific oligonucleotides defined herein hybridise. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

### Fungal Resistance

A genetic modification of the invention can be used to enhance resistance one or more fungal pathogen(s) in a plant such as a wheat plant.

As used herein, “resistance” is a relative term in that the presence of a polypeptide of the invention (i) reduces the disease symptoms of a plant comprising the polypeptide that confers resistance, relative to a plant lacking the gene, and/or (ii) reduces pathogen reproduction or spread on a plant or within a population of plants comprising the gene. Resistance as used herein is relative to the “susceptible” response of a plant to the same pathogen. Typically, the presence of the resistance gene improves at least one production trait of a plant comprising the gene when infected with the pathogen, such as grain yield, when compared to a corresponding wild-type plant infected with the pathogen but lacking the gene. The corresponding wild-type plant may have some level of resistance to the pathogen, or may be classified as susceptible. Thus, the terms “resistance” and “enhanced resistance” are generally used herein interchangeably. Furthermore, a polypeptide of the invention does not necessarily confer complete pathogen resistance, for example when some symptoms still occur or there is some pathogen reproduction on infection but at a reduced amount within a plant or a population of plants. Resistance may occur at only some stages of growth of the plant, for example in adult plants (fully grown in size) and less so, or not at all, in seedlings, or at all stages of plant growth. In an embodiment, resistance occurs at adult and seedling stage. By using a genetic modification strategy to express a polypeptide of the invention in a plant, and/or to increase the level of the polypeptide in a plant, the plant of the invention can be provided with resistance throughout its growth and development. Enhanced resistance can be determined by a number of methods known in the art such as analysing the plants for the amount of pathogen and/or analysing plant growth or the amount of damage or disease symptoms to a plant in the presence of the pathogen, and comparing one or more of these parameters to a corresponding wild-type plant lacking a genetic modification(s) of the invention.

In a particularly preferred embodiment the one or more fungal pathogen(s) causes a disease in the plant such as, but not limited to, stem rust, leaf rust, stripe rust, powdery mildew, head blight, crown rot, foot rot, pink snow mold, spot blotch, common root rot, blast, leaf blight, anthracnose and southern corn blight.

In an embodiment, the one or more fungal pathogen(s) at least infect one or more of the followings plants; wheat, barley, oats, rye, rice, maize or sorghum.

In an embodiment, the one or more fungal pathogen(s) is a Puccinia sp., Blumeria sp., Fusarium sp., Magnoporthe sp., Bipolaris sp., Oidium sp., Gibberella sp., Cochliobolus sp., Exserohilum sp., Uredo sp. Microdochium sp., Helminthosporium sp., Monographella sp., Colletotrichum sp., Uromyces sp. or Erysiphe sp.

In an embodiment, the one or more fungal pathogen(s) is a Puccinia sp., Blumeria sp., Fusarium sp., Magnoporthe sp., Bipolaris sp., Cochliobolus sp., Exserohilum sp. or Erysiphe sp.

In an embodiment, the Puccinia sp. is Puccinia graminis, Puccinia triticina, Puccinia tritici-duri, Puccinia recondita or Puccinia striiformis.

In an embodiment, the Puccinia graminis is Puccinia graminis f. sp. tritici (Ug99).

In an embodiment, the Puccinia recondita is Puccinia recondita f. sp. tritici.

In an embodiment, the Fusarium sp. is Fusarium pseudograminearum, Fusarium graminearum Group II, Fusarium avenaceum, Fusarium culmorum and Fusarium nivale.

In an embodiment, the Blumeria sp. is Blumeria graminis. In an embodiment, the Blumeria sp. is Blumeria graminis f. sp. tritici.

In an embodiment, the Bipolaris sp. is Bipolaris sorokiniana.

In an embodiment, the Gibberella sp. is Gibberella avenacea or Gibberella zeae.

In an embodiment, the Erysiphe sp. is Erysiphe graminis. In an embodiment, the Erysiphe graminis is Erysiphe graminis f. sp. tritici.

In an embodiment, the Exserohilum sp. is Exserohilum turcicum.

In an embodiment, the Magnoporthe sp. is Magnaporthe grisea.

In an embodiment, the Uredo sp. is Uredo glumarum.

In an embodiment, the Microdochium sp. or Microdochium nivale.

In an embodiment, the Monographella sp. is Monographella nivalis.

In an embodiment, the Cochliobolus sp. is Cochliobolus sativus.

In an embodiment, the Helminthosporium sp. is Helminthosporium sativum.

In an embodiment, the Oidium sp. is Oidium monilioides.

In an embodiment, the Colletotrichum sp. is Colletotrichum sublineolum.

In an embodiment, the Uromyces sp. is Uromyces eragrostidis.

### Nucleic Acid Constructs

The present invention includes nucleic acid constructs comprising the polynucleotides of the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in leaves and/or stems of a plant, preferably a cereal plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages such as adults or seedlings. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

In an embodiment, the promoter is a stem-specific promoter, a leaf-specific promoter or a promoter which directs gene expression in an aerial part of the plant (at least stems and leaves) (green tissue specific promoter) such as a ribulose-1,5-bisphosphate carboxylase oxygenase (RUBISCO) promoter.

Examples of stem-specific promoters include, but are not limited to those described in U.S. Pat. No. 5,625,136, and Bam et al. (2008).

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters, tissue specific promoters (see, e.g., U.S. Pat. No. 5,459,252 and WO 91/13992); promoters from viruses (including host specific viruses), or partially or wholly synthetic promoters. Numerous promoters that are functional in mono- and dicotyledonous plants are well known in the art (see, for example, Greve, 1983; Salomon et al., 1984; Garfinkel et al., 1983; Barker et al., 1983); including various promoters isolated from plants and viruses such as the cauliflower mosaic virus promoter (CaMV 35S, 19S). Non-limiting methods for assessing promoter activity are disclosed by Medberry et al. (1992, 1993), Sambrook et al. (1989, supra) and U.S. Pat. No. 5,164,316.

Alternatively, or additionally, the promoter may be an inducible promoter or a developmentally regulated promoter which is capable of driving expression of the introduced polynucleotide at an appropriate developmental stage of the, for example, plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “chimeric vector” is meant a nucleic acid molecule, preferably a DNA molecule derived, for example, from a plasmid, bacteriophage, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (Niedz et al., 1995) or derivatives thereof; a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

The level of a protein of the invention may be modulated by increasing the level of expression of a nucleotide sequence that codes for the protein in a plant cell, or decreasing the level of expression of a gene encoding the protein in the plant, leading to modified pathogen resistance. The level of expression of a gene may be modulated by altering the copy number per cell, for example by introducing a synthetic genetic construct comprising the coding sequence and a transcriptional control element that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in a substantial modification of pathogen resistance or other phenotype. Alternatively, a population of mutagenized seed or a population of plants from a breeding program may be screened for individual lines with altered pathogen resistance or other phenotype associated with pathogen resistance.

### Recombinant Cells

Another embodiment of the present invention includes a recombinant cell comprising a host cell transformed with one or more recombinant molecules of the present invention, or progeny cells thereof. Transformation of a nucleic acid molecule into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing is used to transform the target cell using, for example, targeting nucleases such as TALEN, Cpf1 or Cas9-CRISPR or engineered nucleases derived therefrom are used to cut the DNA and induce changes during cellular repair processes.

A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a cereal plant, more preferably barley or wheat cells, and even more preferably a wheat cell.

### Genome Editing

Endonucleases can be used to generate single strand or double strand breaks in genomic DNA. The genomic DNA breaks in eukaryotic cells are repaired using non-homologous end joining (NHEJ) or homology directed repair (HDR) pathways. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into three major groups using various nucleases or combinations on nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by the synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a U3 or U6 small nuclear RNA promoter. The sgRNA recognises the specific gene and part of the gene for targeting. The protospacer adjacent motif (PAM) is adjacent to the target site constraining the number of potential CRISPR-Cas targets in a genome although the expansion of nucleases also increases the number of PAM's available. There are numerous web tools available for designing gRNAs including CHOPCHOP (http://chopchop.cbu.uib.no), CRISPR design https://omictools.com/crispr-design-tool, E-CRISP http://www.e-crisp.org/E-CRISP/, Geneious or Benchling https://benchling.com/crispr.

CRISPR-Cas systems are the most frequently adopted in eukaryotic work to date using a Cas9 effector protein typically using the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Due to the cell wall on plant cells the delivery of the CRISPR-Cas machinery into the cell and successful transgenic regenerations have used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, accession number KR029109.1).

Alternative CRISPR-Cas systems refer to effector enzymes that contain the nuclease RuvC domain but do not contain the HNH domain including Cas12 enzymes including Cas12a, Cas12b, Cas12f, Cpf1, C2c1, C2c3, and engineered derivatives. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c.

### Sequence Insertion or Integration

The CRISPR-Cas system can be combined with the provision of a nucleic acid sequence to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S. pyogenes Cas9 nuclease with a D10A mutation or H840A mutation.

### Genome Base Editing or Modification

Base editors have been created by fusing a deaminase with a Cas9 domain (WO 2018/086623). By fusing the deaminase can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of Cas9 ribonucleoproteins (RNPs) which are transformed into the cell or protoplast and does not rely on the Cas9 being integrated into the host genome, thereby reducing the undesirable side cuts that has been linked with the random integration of the Cas9 gene. Only short flanking sequences are required to form a stable Cas9 and sgRNA stable ribonucleoprotein in vitro. Woo et al. (2015) produced pre-assembled Cas9/sgRNA protein/RNA complexes and introduced them into protoplasts of Arabidopsis, rice, lettuce and tobacco and targeted mutagenesis frequencies of up to 45% observed in regenerated plants. RNP and in vitro demonstrated in several species including dicot plants (Woo et al., 2015), and monocots maize (Svitashev et al., 2016) and wheat (Liang et al., 2017). Genome editing of plants using CRISPR-Cas 9 in vitro transcripts or ribonucleoproteins are fully described in Liang et al. (2018) and Liang et al. (2019).

### Method for Gene Insertion

Plant embryos may be bombarded with a Cas9 gene and sgRNA gene targeting the site of integration along with the DNA repair template. DNA repair templates are may be synthesised DNA fragment or a 127-mer oligonucleotide, with each encoding the cDNA or the gene of interest. Bombarded cells are grown on tissue culture medium. DNA extracted from callus or TO plants leaf tissue using CTAB DNA extraction method can be analysed by PCR to confirm gene integration. T1 plants selected if pcr confirms presence of the gene of interest.

The method comprises introducing into a plant cell the DNA sequence of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site, the donor DNA is inserted into the transformed plant's genome.

Although homologous recombination occurs at low frequency in plant somatic cells the process appears to be increased/stimulated by the introduction of doublestrand breaks (DSBs) at selected endonuclease target sites. Ongoing efforts to generate Cas, in particular Cas9, variants or alternatives such as Cpf1 or Cms1 may improve the efficiency.

### Genetically Modified Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

As noted above, a genetically modified plant of the invention may be a transgenic plant. A “transgenic plant” as used herein refers to a plant that contains a nucleic acid construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence which may be synthesised in the plant cell or external to the plant cell. Typically, the transgene has been introduced into the plant by human manipulation such as, for example, by transformation but any method can be used as one of skill in the art recognizes. The genetic material is preferably stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”.

A “non-genetically modified plant”, such as a “non-transgenic plant”, is one which has not been genetically modified by the introduction of genetic material by human intervention using, for example, recombinant DNA techniques such as gene editing. In a preferred embodiment, the genetically modified plants are homozygous for each and every gene that has been introduced (such as a transgene) so that their progeny do not segregate for the desired phenotype.

As used herein, the term “compared to an corresponding wild-type plant”, or similar phrases, refers to a plant or grain which comprises at least 75%, more preferably at least 95%, more preferably at least 97%, more preferably at least 99%, and even more preferably 99.5% of the genotype of a plant or grain of the invention but does not have the genetic modification(s) of interest which reduces the pathogenicity of the fungal pathogen on the plant. Preferably, the corresponding wildtype plant is of the same cultivar or variety as the progenitor of the genetically modified plant of interest, or a sibling plant line which lacks the construct, often termed a “segregant”, or a plant of the same cultivar or variety transformed with an “empty vector” construct, and may be a non-genetically modified plant. “Wild type”, as used herein, refers to a cell, tissue, polypeptide or plant that has not been modified according to the invention. Wild-type cells, tissue, polypeptide or plants may be used as controls to compare levels of expression of an exogenous nucleic acid or the extent and nature of trait modification with cells, tissue or plants modified as described herein. In an embodiment, the wild-type plant is an isogenic plant lacking the genetic modification (s).

Genetically modified plants, as defined in the context of the present invention include progeny of the plants which have been genetically modified, wherein the progeny comprise the genetic modification of interest. Such progeny may be obtained by self-fertilisation of the primary genetically modified plant or by crossing such plants with another plant of the same species. This would generally be to modulate the production of at least one protein defined herein in the desired plant or plant organ. Genetically modified plant parts include all parts and cells of said plants comprising the genetic modification such as, for example, cultured tissues, callus and protoplasts.

Plants contemplated for use in the practice of the present invention include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plant is a cereal plant, more preferably wheat, rice, maize, triticale, oats or barley, even more preferably wheat.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species.

Also encompassed are plants that are produced by conventional techniques using Triticum sp. as a parent in a sexual cross with a non-Triticum species (such as rye [Secale cereale]), including but not limited to Triticale.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare or suitable for commercial production of grain.

Genetically modified plants, as defined in the context of the present invention include plants (as well as parts and cells of said plants) and their progeny which have been genetically modified using recombinant techniques to cause production of at least one polypeptide of the present invention in the desired plant or plant organ. Genetically modified plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the genetically modified plants are homozygous for each and every genetic modification that has been introduced (such as a transgene) so that their progeny do not segregate for the desired phenotype. The transgenic plants may also be heterozygous for the introduced genetic modifications(s), such as, for example, in F1 progeny which have been grown from hybrid seed. Such plants may provide advantages such as hybrid vigour, well known in the art.

As used herein, the “other genetic markers” may be any molecules which are linked to a desired trait of a plant. Such markers are well known to those skilled in the art and include molecular markers linked to genes determining traits such disease resistance, yield, plant morphology, grain quality, dormancy traits, grain colour, gibberellic acid content in the seed, plant height, flour colour and the like. Examples of such genes are the rust resistance genes mentioned herein, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different genetically modified plants can also be mated/crossed to produce offspring that contain two independently segregating exogenous genes. Selfing of appropriate progeny can produce plants that are homozygous for both exogenous genes. Back-crossing to a parental plant and out-crossing with a non-genetically modified plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of polynucleotides such as DNA into plants by direct transfer into pollen, by direct injection of polynucleotides such as DNA into reproductive organs of a plant, or by direct injection of polynucleotides such as DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Genetically modified embryos and seeds are similarly regenerated. The resulting genetically modified rooted shoots are thereafter planted in an appropriate plant growth medium such as soil.

The development or regeneration of plants containing the genetic modification(s) is well known in the art. Preferably, the regenerated plants are self-pollinated to provide homozygous genetically modified plants. Otherwise, pollen obtained from the regenerated plants is crossed to seed-grown plants of agronomically important lines. Conversely, pollen from plants of these important lines is used to pollinate regenerated plants. A genetically modified plant of the present invention containing a desired genetic modification is cultivated using methods well known to one skilled in the art.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. Nos. 5,569,834, 5,416,011); Brassica (U.S. Pat. No. 5,463,174); peanut (Cheng et al., 1996); and pea (Grant et al., 1995).

Methods for transformation of cereal plants such as wheat, maize and barley for introducing genetic modification(s) into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. Nos. 5,589,617, 6,541,257, and other methods are set out in WO 99/14314. In an embodiment, transgenic cereal plants such as wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the genetic modification in cells and plants, a polymerase chain reaction (PCR) amplification, sequencing, or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once genetically modified plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Marker Assisted Selection

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis of at least one polynucleotide/polypeptide of the invention that confers upon the plant resistance to one or more fungal pathogen(s), allows rapid selection of plants carrying the desired trait, which may be nurtured to maturity in the greenhouse or field for subsequent further backcrossing to the recurrent parent.

Any molecular biological technique known in the art can be used in the methods of the present invention. Such methods include, but are not limited to, the use of nucleic acid amplification, nucleic acid sequencing, nucleic acid hybridization with suitably labelled probes, single-strand conformational analysis (SSCA), denaturing gradient gel electrophoresis (DGGE), heteroduplex analysis (TET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of the (for example) polynucleotide and/or genetic modification of the invention which confers upon the plant resistance to one or more fungal pathogen(s). Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al., (2001).

In an embodiment, a linked loci for marker assisted selection is at least within 1 cM, or 0.5 cM, or 0.1 cM, or 0.01 cM from a gene encoding a polypeptide of the invention.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G Moller (editors), BIOS Scientific Publishers Ltd, Oxford, (2000)). PCR can be performed on cDNA obtained from reverse transcribing mRNA isolated from plant cells comprising a polynucleotide and/or genetic modification of the invention which confers upon the plant resistance to one or more fungal pathogen(s). However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al., (supra) and Sambrook et al., (supra). Sequencing can be carried out by any suitable method, for example, dideoxy sequencing, chemical sequencing or variations thereof. Direct sequencing has the advantage of determining variation in any base pair of a particular sequence.

### Tilling

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Plant/Grain Processing

Grain/seed of the invention, preferably cereal grain and more preferably wheat grain, or other plant parts of the invention, can be processed to produce a food ingredient, food or non-food product using any technique known in the art.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain such as wheat or barley grain. The particle size of refined flour is described as flour in which not less than 98% passes through a cloth having openings not larger than those of woven wire cloth designated “212 micrometers (U.S. Wire 70)”. The coarse fraction includes at least one of bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e. -ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively, bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain such as barley and wheat grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:
(i) providing grain, such as barley or wheat grain, of the invention,
(ii) steeping said grain,
(iii) germinating the steeped grains under predetermined conditions and
(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of specialty malts, including, but limited to, methods of roasting the malt.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

### EXAMPLES

### Example 1—Material and Methods

### Flow Sorting and Sequencing of Chromosome 3B of Marquis and Hope

Marquis 3B and Hope sequencing has been described in De Oliveira et al. (2020). In general, flow cytometry was used to sort chromosome 3B DNA following the protocol detailed in (Vrána et al., 2000). Sorted chromosomal DNA was then amplified as previously described in (Šimková et al., 2008) to generate sufficient amount of DNA for sequencing. DNA samples were then sequenced with Illumina HISEQ-2000 using 2×100 bp paired-end reads (ENA project number PRJEB31708).

Adaptor and quality trimmed sequences of Marquis 3B were aligned to the Sr2 region sequence of Hope 3B chromosome (GenBank accession no. KP244323.1) by CLC Genomics Workbench version 11.0.1 (Qiagen, USA), to identify sequence variations between the two haplotypes.

### Mapping of Sr2 in Marquis×CS(Hope3B) Cross

A F2 mapping family was developed by crossing the susceptible wheat cv Marquis with the resistant cv. CS(Hope3B). 5000 F2 seeds were used for high resolution mapping. DNA was prepared from half seeds using the protocol described by Ellis et al. (2005) and screened with SNP based KASP markers identified from sequence comparison of the region between Hope and Marquis (see previous section). This included MST_2, CD882879 and a SNP marker (wMAS000005) based on CSSr2 (Mago et al., 2011b) (https://maswheat.ucdavis.edu/protocols/Sr2). KASP assays were carried out according to manufacturer's protocol (http://info.biosearchtech.com) on a BIO-RAD CFX-96 qPCR machine (www.Bio-Rad.com).

Recombinants obtained from the initial screen were confirmed by repeating the KASP assay and the remaining half seed of the recombinant was grown. 24 progeny seeds from each recombinant were germinated and screened with the respective markers to identify and obtain homozygous lines.

### Long-Read BAC Sequencing and Generation of Physical Map of Sr2 Locus

Based on the position of new recombinants flanking the Sr2 locus, the inventors used 12 BACs which span this region (Mago et al., 2014) for re-sequencing using the MinION platform of Oxford Nanopore Technologies.

### DNA Extraction, Purification and Size Selection

Escherichia coli containing BACs were grown in a 15 mL Falcon tube containing 5 mL Luria Broth (Lennox) (Sigma) with 12.5 mg/mL Chloramphenicol. Tubes were incubated at 37° C., shaking at 400 rpm, for approximately 16 h. High-molecular weight DNA was extracted according to Mayjonade et al. (2016) with some modifications. The bacterial pellet was first treated with 5 mg/mL lysozyme in a 100 μL volume at 55° C. for 20 min. Protocol was then resumed as normal. Briefly, the bacterial suspension was then incubated with an SDS based lysis buffer, proteins precipitated with potassium acetate, DNA bound to Sera-Mag™ SpeedBead magnetic carboxylate-modified particles (GE Healthcare), washed several times with 70% ethanol and eluted with 10 mM Tris-HCl pH 8. DNA was further purified and size selected according to Jones et al. (2020). This included further protein removal, RNA removal, clean-up with cholorform:isoamyl alcohol (24:1), and 10 kb+ size selection with a Short Read Eliminator (SRE) XS (Circulomics).

### DNA Sequencing

To perform native DNA sequencing, Oxford Nanopore Technologies (ONT) portable MinION Mk1B was adopted. Sequencing libraries were constructed according to the manufacturer's protocol; native barcoding genomic DNA (with EXP-NBD104 and SQK-LSK109), version NBE_9065_v109_revV_14aug.2019. Briefly, approximately 1 ug DNA for each BAC sample was repaired (FFPE DNA Repair Mix, New England BioLabs® (NEB)), end-prepped with an adenosine overhang (Ultra II end repair/dA-tailing module, NEB), purified (AMPure XP, Beckman Coulter) and a unique ONT native barcode was ligated to each BAC (Quick T4 Ligation Module, NEB). Following this, each sample was cleaned again (AMPure XP), quantified using a Qubit Fluorometer (Thermo Fisher Scientific), pooled in equimolar amounts, an ONT adapter was ligated, the library was cleaned and quantified again. A MinION FLO-MIN106 9.4.1 revD flow cell was primed, approximately 300 ng of the pooled library was loaded and sequenced according to the manufacturer's instructions (ONT).

### Bioinformatic Processing and De Novo Assembly

Raw fast5 reads were processed with Guppy v3.4.5 (ONT), which performed basecalling (high accuracy config file dna_r9.4.1_450bps_hac), demultiplexing (—barcode_kits EXP-NBD104) and the removal of barcode/adapter sequences (—trim barcodes). Sequencing output and quality was inspected with the NanoPack tool NanoPlot v1.28.2 (De Coster et al., 2018). Reads were then filtered using NanoPack tool NanoFilt v2.6.0 (De Coster et al., 2018), selecting reads of minimum length 20 kb and minimum quality 10. The filtered reads were subjected to de novo assembly using Unicycler v0.4.9b (Wick et al., 2017). The long-read-only assembly was used with default parameters, which includes multiple rounds of read correction with Racon v1.4.11 (Vaser et al., 2017). Illumina reads used for physical map of the region previously (Mago et al., 2014; GenBank accession no. KP244323) were used for polishing.

### Mapping Analyses

BAC vector sequences were mapped to the Unicycler assemblies using Minimap2 v2.17-r941 (Li, 2018) and the unique insert sequences were extracted and aligned to each other to build an overlapping contig using Sequencher v5.3 and confirmed manually. Gene annotation was done using FGNESH (www.softberry.com) and confirmed using RNAseq and BLAST analysis. To identify any regions of duplication a dot plot analysis was done by comparing the sequence to itself using YASS (https://bioinfo.lifl.fr/yass/index.php).

### Gene Expression Analysis

Adults plants of CS(Hope3B) were infected with Pgt 98-1,2,3,5,6 and flag-1 leaf samples from 3 independent plants were collected at 0 and 48 hours post infection and immediately snap frozen in liquid Nitrogen and stored at −80° C. Total RNA was isolated using Qiagen RNeasy Plant Mini Kit as described earlier (Mago et al., 2014). RNA samples were sequenced by Genewiz (www.genewiz.com). RNA-seq was quality checked using Fastqc and low-quality bases and reads were trimmed using Trimmomatic. An index of the trimmed reads was prepared by using the CS(Hope3B) sequence of the Sr2 locus and the housekeeping gene TaGAPDH. Alignment of the sequences and visualisation was done in Samtools. Reads for all the samples were and extracted and normalised against TaGAPDH, normalized transcript was represented as Reads Per Kilobase of transcript, per Million mapped reads (RPKM).

For qRT-PCR analysis of gene expression plants were infected with Pgt race 21-0 at anthesis. Flag-1 leaf was collected at 0 and 48 hrs post infection and immediately snap frozen using liquid nitrogen. RNA extraction was done using RNeasy kit (www.Qiagen.com) according to manufacturer's instructions. Quantitative PCR was carried out on a Bio-Rad CFX96 Touch Real-Time PCR Detection System (Bio-Rad) using iTaq universal SYBR Green supermix (Bio-Rad) and a two-step cycling program according to the manufacturer's instructions and as described in Moore et al., (2015). Minus RT controls were first tested with housekeeping gene TaCON (Moore et al., 2015) to ensure amplification of residual genomic DNA was insignificant. Quantitative RT-PCR was done on RNA isolated from leaf tissue infected with powdery mildew at 4 leaf stage and sheath infected with stem rust at anthesis. Primers used for quantitative PCR (qPCR) for D8LAL2 (TaPMP-B4) and CD882879 (TaPMP-B3a; PMHo F2×PMHo R2) are listed in Table 2. TaPMP-B3b (CD882879) expression in Marquis transgenics was assessed using primers PMHoF1×PMHoR1. TaPMP-B3 expression in CS was detected using primers CS3B-PMP3-qpcrF×CS3B-PMP3-qpcrR and primers CS3B-PMP4-qpcrF and CS3B-PMP4-qpcrR were used for TaPMP-B4 expression (Table 2). Primers for TaCON are described in Moore et al. (2015) and provided in Table 2. The green channel was used for data acquisition. Efficiency and cycle threshold values were calculated using the LinRegPCR quantitative PCR data analysis (Rujiter et al., 2009), and relative expression levels were calculated using the Relative expression software tool (REST) method (Pfaffl et al., 2002) relative to the housekeeper gene, TaCON.

TABLE 2
Primer sequences.

| | | Size |
|---|---|---|
| Primer name | Primer sequence | bp |
| MSF_2F | CATTCGTGCGTAAGCATGGA | |
| | CTAAT | |
| | (SEQ ID NO: 38) | |
| MSF_2V | CATTCGTGCGTAAGCATGGA | |
| | CTAAA | |
| | (SEQ ID NO: 39) | |
| MSF_C | ATGCATTCGTACATTTTTCT | |
| | GCCACTGAT | |
| | (SEQ ID NO: 40) | |
| PMPstgF | GAACCGTTCCCGTGGATT | |
| | (SEQ ID NO: 41) | |
| PMPstgR | CGGATTTGAAGATAATGAAG | |
| | TGTG | |
| | (SEQ ID NO: 42) | |
| CD882879_F2 | GGGAACGGTTCAATAGTCCA | |
| | T | |
| | (SEQ ID NO: 43) | |
| CD882879_V2 | GGGAACGGTTCAATAGTCCA | |
| | A | |
| | (SEQ ID NO: 44) | |
| CD882879_C5 | CATGACGTCATGTTGGGGGT | |
| | TC | |
| | (SEQ ID NO: 45) | |
| Sr2_DupJn-F1 | CCTTTTTGCGACAAGTCGAG | |
| | (SEQ ID NO : 46) | |
| Sr2_DupJn-F2 | TCTCTCACCATGTGGCTCTG | 502 |
| | A | |
| | (SEQ ID NO : 47) | |
| Sr2_DupJn-R1 | CAGATCCGAAGGCAAAACAT | |
| | (SEQ ID NO: 48) | |
| Sr2_DupJn-R2 | AAGACATCAAACGGCAGGAG | |
| | (SEQ ID NO: 49) | |
| D8LAL2-F2_qPCR | TACTGATAGTGGCGGCAGTG | 200 |
| | (SEQ ID NO: 50) | |
| D8LAL2-R1_qPCR | GGTAGCTACCATGCTCATAA | |
| | TTGG | |
| | (SEQ ID NO: 51) | |
| PMHo_F2_qPCR | TTCCCGAGAAGTAGCGTGAT | 168 |
| | C | |
| | (SEQ ID NO: 52) | |
| PMHo_R2_qPCR | TGAGCTATCATACAAGTAGC | |
| | AGACG | |
| | (SEQ ID NO: 53) | |
| PMP3Oex-F | ACAGGATCCTGTCCAGATCT | 2207 |
| | CAACCACAC | |
| | (SEQ ID NO: 54) | |
| PMP3Oex-R | TCAGGTACCAGTAGTTTTGT | |
| | TGCCAACTG | |
| | (SEQ ID NO: 55) | |
| PMP-UB-VNF | GCTAGTACTGCCAGCGGTAA | 150 |
| | A | |
| | (SEQ ID NO: 56) | |
| PMP-UB-VNR | CATTGCCGAAAAATCCTGTT | |
| | (SEQ ID NO: 57) | |
| PMPstgF | GAACCGTTCCCGTGGATT | 223 |
| | (SEQ ID NO: 58) | |
| pStg-PMPsp | GATTGCCAGGATCACGCTAC | |
| | TTCTCG | |
| | (SEQ ID NO: 59) | |
| pStg_D8LAL-F | GTGAAAAATCGGATGTAACG | 186 |
| | TCG | |
| | (SEQ ID NO: 60) | |
| pStg-D8LALsp | CGTTCGAAGGCGCTAATGTG | |
| | CTC | |
| | (SEQ ID NO: 61) | |
| D8LAL2-shF2 | ACGAAGGCCAATTGATTGAC | 2953 |
| | (SEQ ID NO: 62) | |
| D8LAL2-shR | GGCAAAATGATCTTGGGTGT | |
| | (SEQ ID NO: 63) | |
| PMP3b_DupJnF | TCTCTCACCATGTGGCTCTG | 502 |
| | A | |
| | (SEQ ID NO: 64) | |
| PMP3b_DupJnR1 | CAGATCCGAAGGCAAAACAT | |
| | (SEQ ID NO: 65) | |
| Sr2DJn1FAM | GAAGGTGACCAAGTTCATGC | |
| | TGGACCCTACTCTGGAAGAC | |
| | CTAA | |
| | (SEQ ID NO: 66) | |
| Sr2DJn1Vic | GAAGGTCGGAGTCAACGGAT | |
| | TGGACCCTACTCTGGAAGAC | |
| | CTAG | |
| | (SEQ ID NO: 67) | |
| Sr2DJnComR1 | ACCTCGTAGGTCTTCCAGAG | |
| | TAG | |
| | (SEQ ID NO: 68) | |
| TaCON-F | GTGATACAGGGGGCGCAGAG | |
| | (SEQ ID NO: 69) | |
| TaCON-R | GTGGCTTCCGAAATAGAGTA | |
| | G | |
| | (SEQ ID NO: 70) | |
| CS3B-PMP3-qpcrF | TGATTGCTTCATGGCGGG | 79 |
| | (SEQ ID NO: 72) | |
| CS3B-PMP3-qpcrR | GGGCAACCATCTCAAGCTG | |
| | (SEQ ID NO: 73) | |
| CS3B-PMP4-qpcrF | CTCAGCCTTAACCTCGACCG | 96 |
| | CCCG | |
| | (SEQ ID NO: 74) | |
| CS3B-PMP4-qpcrR | TACACCTAGGGCAATAGTGA | |
| | GTCCCTTCATGTGTC | |
| | (SEQ ID NO: 75) | |
| UbiF1 | TGCAGCATCTATTCATATGC | 473 |
| | TCT | |
| | (SEQ ID NO: 76) | |
| D8LAL_FendR | AAAGAGAGTCGAGATAGACT | |
| | TACC | |
| | (SEQ ID NO: 77) | |
| Can425 | TGCTTCTCGCACTTGCTCTA | |
| | (SEQ ID NO: 78) | |
| Can426 | AAGCAGCACACCATGAAGG | |
| | (SEQ ID NO: 79) | |
| SR2-P2 | TCCCCATTGCCTTGATTGCC | |
| | A | |
| | (SEQ ID NO : 80) | |
| TaPMP- | ggggacaagtttgtacaaaa | |
| | aagcaggcttaATGGTACGT | |
| | ACTAATTCC | |
| 3B_attb1 | TCTTTATC | |
| | (SEQ ID NO: 81) | |
| TaPMP-3Bwostop_ | ggggaccactttgtacaaga | |
| attb2 | aagctgggtcTTTACCGCTG | |
| | GCAGTACTA | |
| | GCCATTG | |
| | (SEQ ID NO: 82) | |
| TaPMP-B4_attb1 | ggggacaagtttgtacaaaa | |
| | aagcaggcttaATGGAGTGG | |
| | AGCCAAGGA | |
| | TCTCAG | |
| | (SEQ ID NO: 83) | |
| TaPMP-B4wostop_ | ggggaccactttgtacaaga | |
| attb2 | aagctgggtcTAGTGGTTGT | |
| | TGGAGATCA | |
| | AGCCGG | |
| | (SEQ ID NO: 84) | |

For RNA gel blot analysis of gene expression in silenced plants, leaf tissue (flag minus 1) from adult plants at anthesis was collected at 48-hours post inoculation with Pgt 98-1,2,3,5,6. RNA was extracted according to Pattemore (2014). For the RNA gel blot, 10 μg of total RNA per sample were separated on an 1% agarose gel (buffer) and transferred onto a Hybond N+ membrane (GE life sciences, USA). The probes used for hybridization were amplified using primers PMPstgF×pStg-PMPsp (TaPMP-B3) and pStg_D8LAL-F×pStg_D8LALsp (TaPMP-B4), respectively (Table 2), labelled with 32P-UTP. Membranes were washed twice with 2×SSC (Saline sodium citrate buffer) buffer at 65oc for 20 min each followed by 3 washes of 10 min each with 2×SSC at room temperature. The membrane was exposed to a phosphor screen and read using a Typhoon FLA 9500Phosphorimager (Cytiva, USA).

### Gene Copy Number Analysis

Copy number analysis was performed using Digital Droplet PCR (Bio-Rad, Australia) according to the protocol described in Petrie et al. (2020). Genomic DNA was isolated from leaves of CS, CS(Hope3B), Hope and Marquis according to Lagudah et al. (1991). Wheat Zinc Finger protein gene Hd1 which is a homolog of barley CONSTANS-like CO2 gene was used as a reference gene control. Primers (Can425, Can426) and probe primer (SR2-P2) used for copy number analysis of TaPMP-B3 are listed in Table 2. The inventors also compared the coverage of sequence reads from the 3B chromosome sorted from CS(Hope3B) and Marquis (see above, De Oliveira et al. 2020) at the Sr2 locus to determine copy number of genes. Illumina reads derived from the isolated chromosomes were aligned to the Hope3B reference sequence across the Sr2 locus (˜1 Mbp) using BWA-MEM with default parameters and read coverage per base determined using SAMTOOLS depth.

### Vector Construction

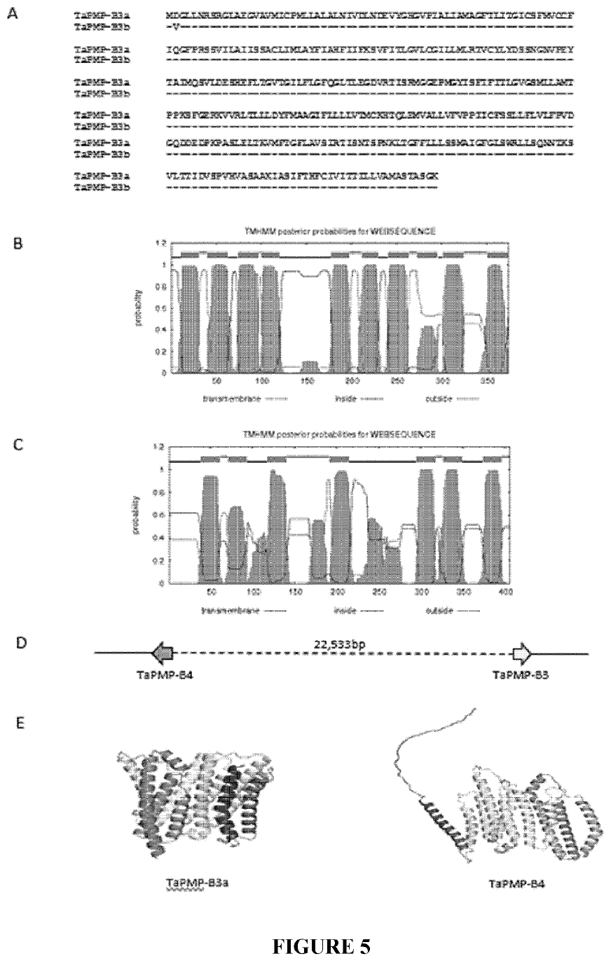

A 300 bp genomic fragment of TaPMP-B3a (CD882879) and TaPMP-B4 (D8LAL2), respectively (Example 10) were synthesised with flanking attB sites commercially (Invitrogen GeneArt Gene Synthesis, Thermo Fisher Scientific, MA, USA). The fragments were cloned into pENTR™/D-TOPO® according to manufacturer's (Invitrogen™, Thermo Fisher Scientific, MA, USA) instructions and finally cloned into pSTARGATE vector (Greenup et al., 2010) using Gateway® Technology (Life Technologies™). FIG. 5 shows the hairpin (Hp) vector map generated for PMP3 (CD882879) which was used for transformation. Hairpin vector for TaPMP-B4 was similarly made. All plasmids were confirmed by sequencing and analyzed using Vector NTI Advance (Life Technologies, Thermo Fisher Scientific, MA, USA) or Sequencher v5.3 (Gene Codes Corporation, MI, USA) software.

For complementation, coding sequences of TaPMP-B3a and TaPMP-B4 including an intron were PCR amplified from the resistant wheat CS(Hope3B) using primers PMP30ex-F/PMP30ex-R and D8LAL2-shF2/D8LAL2-shR, respectively (Table 2). The TaPMP3-B3a fragment was digested with the restriction enzymes BamHI and KpnI. The fragment was cloned into pWUbi.tm1 (Wang et al., 1998) digested with BamHI and KpnI. Positive clones were further digested with NotI and the Ubi-TaPMP-B3a fragment was cloned into Agrobacterium transformation vector pVecNeo (Cesari et al., 2016).

The TaPMP-B4 fragment was cloned into pGEMT-easy vector (Promega, USA) and digested with NotI. Plasmid pWubi.tm1 was initially digested with SacI and religated to remove tm1. The remaining plasmid carrying Ubi promoter was cloned into transformation vector Vec8 (Wang et al., 1997) as a HindIII-NotI fragment. The TaPMP-B4 was cloned into Vec8-Ubi as a NotI fragment. The vector maps were prepared using Vector NTI Advance (Life Technologies, Thermo Fisher Scientific, MA, USA). Plasmids were sequenced and analysed using Sequencher v5.3 (Gene Codes Corporation, MI, USA) software. Wheat cv Marquis was transformed using the Agrobacterium tumefaciens strain GV3101 (pMP90) with pVecNeo-Ubi-CD882879 as described (Richardson et al., 2014). Transformants were tested for the presence of transgene by PCR using primers PMP-UB-VNF and PMP-UB-VNR (Table 2). Vec8-Ubi-TaPMP-B4 was transformed into a homozygous Marquis transgenic line carrying VecNeo-Ubi-TaPMP-B3a and hygromicin was used as selection. Transformants were tested for presence of the transgene using primers UbiF1 and D8LAL_FendR (Table 2).

For transient expression in planta, the coding sequences including introns of TaPMP-B3a, TaPMP-B3b and TaPMP-B4 were PCR amplified (primer pairs TaPMP-3B_attb1/TaPMP-3Bwostop_attb2 and TaPMP-B4_attb1/TaPMP-B4wostop_attb2 respectively) and cloned into the pDONR207 vector using Gateway cloning (Invitrogen). The predicted coding sequences including introns of TaPMP-B3CS and TaPMP-B4CS were synthesized and subcloned into pDONR207 similarly. Gene sequences were then transferred into the binary vector pAM-35s-GWY-YFPv by Gateway cloning as previously described by Bernoux et al. (2008). Primers details are listed in Table 3.

Transient Protein Expression in N. benthamiana and N. tabacum

N. benthamiana and N. tabacum plants were grown in a growth chamber at 23° C. with a 16-h light period. Agrobacterium cultures containing the expression vectors of each construct were grown overnight at 28° C. in LB media with appropriate antibiotic selections. The cells were pelleted and resuspended in infiltration mix (10 mM MES pH 5.6, 10 mM MgCl2, 1500 μM acetosyringone) to an optical density (OD600) of 0.5 or 1.0, followed by incubation at room temperature for 2 h. Cultures were infiltrated into leaves of 4-week-old tobacco with a 1-ml syringe. For documentation of cell death, leaves were photographed or scanned 2-5 d after infiltration. For LaCl3 treatment experiment, 2 mM LaCl3 was added into the infiltration mix and applied to N. benthamiana or N. tabacum.

### Rust, and Powdery Mildew Infection and Other Phenotyping

Adult plant stem rust infection assays were done as described previously (Mago et al., 2014; Tabe et al., 2019). Briefly, rust spores of Pgt race 98-1,2,3,5,6 or 21-0 were mixed with talcum powder (1:2) and inoculated onto sheaths and leaves of plants at anthesis. Inoculated plants were incubated in a humid chamber for 48 hrs at 23oc and moved to a growth cabinet maintained at 23° C. day and 18° C. night with a 16 hrs light and 8 hrs dark cycle. Rust phenotyping was done several times from 9-15 days post infection. In experiments which included recombinants and parents of the CS(Hope3B) and Marquis mapping family and Marquis transgenics pgt 21-0 was used as pgt 98-1,2,3,5,6 is avirulent on Marquis.

Seedling leaf rust tests were done with Pt race 122-1,2,3,5 (PBI #351) as described (McIntosh et al., 1995) and plants were scored at 14 dpi according to the Stakman scale (Stakman et al., 1962). Powdery mildew phenotyping was done according to Tabe et al. (2019), in short four-week-old plants were inoculated with mildew by applying spores of a glasshouse isolate of Bgt to the abaxial surface of the leaves by shaking infected leaves over them, followed by application of a fine spray of water. Plants were maintained under a shade curtain in the glasshouse at 23° C., in tubs of shallow water to increase humidity. Plants were scored 9-14 dpi post inoculation. Vaseline and heat shock induced necrosis were done as described by Tabe et al. (2019).

### Rust Biomass Assays

In planta fungal biomass quantification was done by Wheat germ Agglutinin Chitin (WAC) assays according to Ayliffe et al. (2014). Three infected leaf sheaths from plants infected with Pgt were removed 9 dpi, pooled, weighed, cut into 1-2 cm fragments and stored at −80oC until material from all the plants had been collected. The sheath tissue was freeze dried in 2 ml Eppendorf tubes, crushed into a fine powder using Qiagen TissueLyser II (www.Qiagen.com) and powder transferred to 15 ml falcon tubes. After adding 4 ml 1M KOH, each sample was incubated at 65° C. with occasional mixing for 96 hrs. The tubes were centrifuged at 3000 rpm and pellet washed with 50 mM Tris-HCl pH 7.5 and the process repeated 4 times. Samples were suspended in 50 mM Tris-HCl pH 7.5 and sonicated for 20 seconds. OD 600 nm was measured and samples diluted to a reading of 1.0 to 1.1. Each sample was divided into 4 (250 μl each) and fluorescence was measured using FLUOstar Omega plate reader (BMG LABTECH, Germany). Means of the technical replicate fluorescence values were calculated.

### Sequence Analysis of Candidate Genes

The amino acid sequence comparison of CD882879 (TaPMP-B3) between CS(Hope3B) (TaPMP-B3a) and Marquis (TaPMP-B3b) was done using Clustal Omega (https://www.ebi.ac.uk). Transmembrane domain prediction was done using TMHMM Server v. 2.0 (http://www.cbs.dtu.dk/services/TMHMM/).

The monomer and multimer protein structure predictions of TaPMP-3Ba (wheat cv Chinese Spring, SEQ ID NO:85) and TaPMP-B4 (wheat cv Chinese Spring, SEQ ID NO:86) and homologs were performed using alphafold V2.1.1 (https://github.com/deepmind/alphafold). Searches for related protein structures and comparisons of predicted structures were conducted using the dali server (ekhidna2.biocenter.helsinki.fi/dali/) (Holm, 2020).

### Example 2—Marquis 3B Sequencing and Mapping of Sr2 Resistance

Previously, the locus conferring Sr2 resistance was mapped using a CS×CS(Hope3B) family (Mago et al., 2014) between the recombining markers RKO_1 and DOX 1, which are approx 1.0 Mb apart on the physical map of Chinese Spring. However, at this locus the inventors found substantial structural variation between the 3B haplotypes of CS and Hope.

Markers developed from BAC ends of CS BACs spanning the region were used to screen BAC libraries prepared from flow sorted 3B chromosome of Hope (Mago et al., 2014). A BAC contig spanning the locus (between the recombining markers) was developed and sequenced using Illumina short-read sequencing (GenBank accession no. KP244323). Genes were annotated using gene prediction software packages and alignment of RNA-seq reads from leaves of cv Hope with the Sr2 genomic sequence. In total, 34 putative genes were annotated within this region in cv Hope, by comparison only 17 had been annotated in CS (Mago et al., 2014). This sequence comparison identified a large insertion carrying several copies of Germin-like proteins (Ta-GLP3) in 3B-Hope vs the corresponding region in CS-3B. Further analysis however indicated the presence of the Ta-GLP3 family at this locus in several other susceptible wheats including Marquis. A SNP between Hope and Marquis in Ta-GLP3-8/9 was used to develop a PCR marker for Sr2 (Mago et al., 2011b). Although the Ta-GLP3-8/9-derived marker is tightly linked to Sr2 phenotype, the marker was separated from the locus by recombination. Given the important role of Sr2 in wheat breeding, a project was initiated to identify the causal gene.

The susceptible cultivar Marquis contained a similar haplotype structure to CSHope3B. Therefore, the inventors used this line to support further fine resolution mapping within this region. The inventors used flow cytometry to isolate the 3B chromosome from Marquis which was sequenced using Illumina technology. The sequences were trimmed and used for de novo assembly and compared to the Sr2 sequence from the resistant haplotype in Hope (Mago et al., 2014). This analysis showed that Marquis and Hope were very similar and only a few SNPs were identified, including a SNP within MSF_2 (Mago et al., 2014) and a predicted gene TaPMP-B3a (CD882879) and the previously identified SNP used to design the Sr2 marker CSSr2 (Mago et al., 2011b). These SNPs were converted to KASP markers.

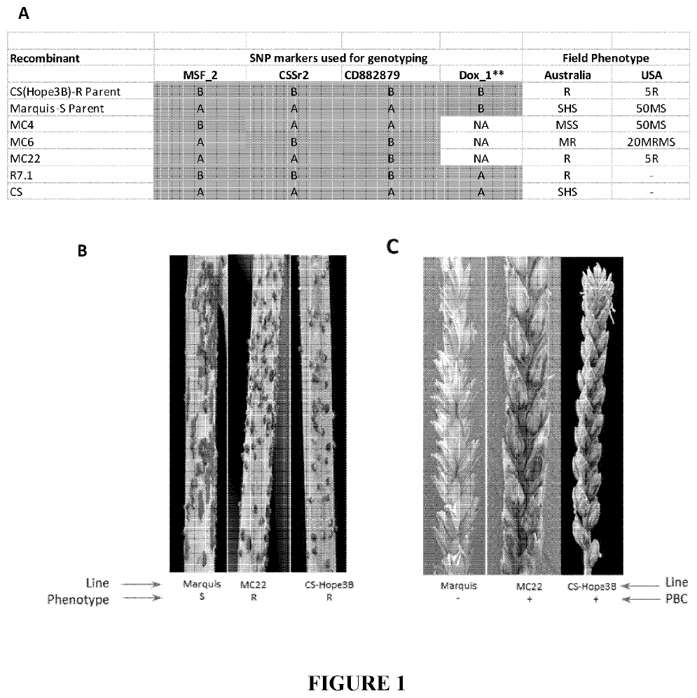

To further delineate the Sr2 locus, the inventors generated a cross between the resistant CS(Hope3B) and Marquis and screened approx. 5000 F2s with KASP markers derived from MSF_2, CSSr2 (wMAS000005, https://maswheat.ucdavis.edu/protocols/Sr2) and a SNP based on TaPMP-B3a (CD882789) (Table 2). This identified a total of 22 recombinants, which were screened for stem rust infection phenotypes in several field sites. A subset of three recombinants are shown in FIG. 1A that represent the major recombination breakpoints. In 21 recombinants, the breakpoints occurred between MSF_2 and CSSr2 (eg MC4 and MC6), however in one recombinant (MC22) the breakpoint was distal to the CSSr2 marker (FIG. 1A). FIGS. 1 B and C shows the rust and PBC phenotype of the critical recombinant MC22 compared to the parental response.

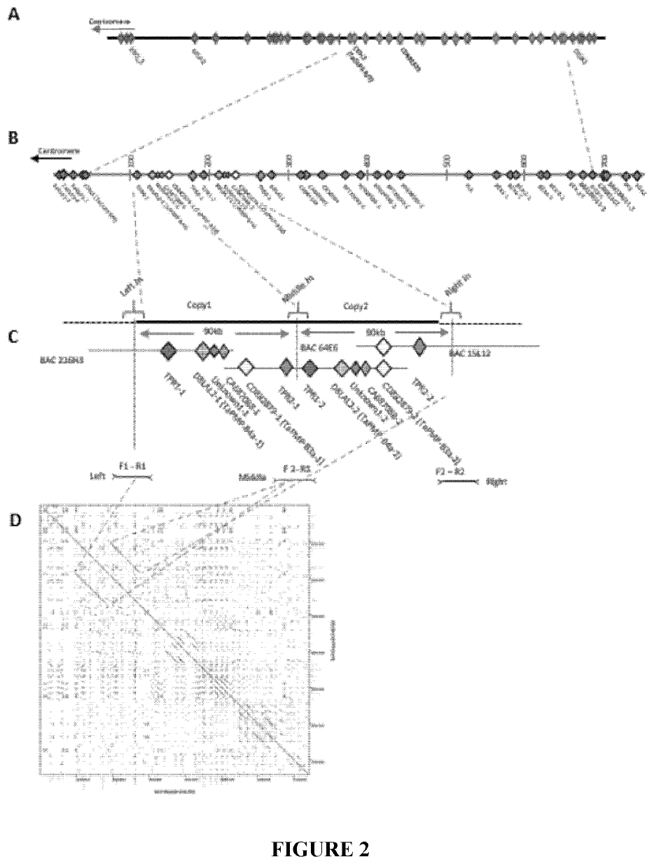

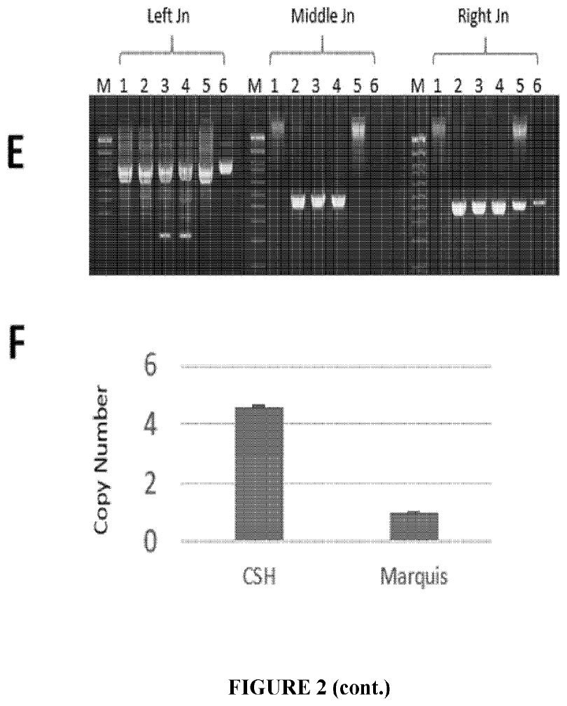

The marker DOX_1 was not polymorphic between the parents in this cross but defined the distal breakpoint in recombinant R7.1 in the previous mapping population derived from the CS×CS(Hope3B) cross (Mago et al., 2014). FIG. 2A shows the position of the informative Sr2 locus markers used on the previously published physical map of the region (Mago et al., 2014). Combining the map information from both populations, it was determined that the Sr2 resistance locus is located between the markers CSSr2 and DOX_1, thereby reducing the physical region carrying the gene from 1.0 Mb to approximately 0.6 Mb (FIG. 2B).

### Example 3—Identification of Duplicated Regions at Sr2 Locus Using Long Read Sequencing and Copy Number Analysis

Previously, the Sr2 region sequence was assembled from Illumina-based sequencing of overlapping BAC clones (Mago et al., 2014). To confirm the assembly and gene content of this region, twelve overlapping clones spanning the new physical interval were re-sequenced using Nanopore sequencing on the MinION platform of Oxford Nanopore Technologies, which produces reads that can reach tens or even hundreds of kb (Tulpovi et al., 2019). Combining the individual overlapping BAC sequences resulted in an overall assembly of 741,783 bp spanning the Sr2 region (FIG. 2B). The re-sequenced region was annotated by sequence comparison to previously published gene content and by de novo annotation (Mago et al., 2014). In total 39 genes were annotated in the overlapping BAC sequences (Table 3), of these 29 were contained within a sequence of approx 0.64 Mb flanked by the recombining markers. Table 4 lists the size and positions of duplications above 15 kb in size. The largest of these duplications was approximately 90 kb and included six genes: TPR_1, D8LAL2 (TaPMP-B4), CA687088, CD882879 (TaPMP-B3), TPR_2 and a gene with unknown function that was previously not annotated (FIG. 2C). This duplication had not been detected in the previous assembly because BAC contigs were assembled using short-read sequences. FIG. 2D shows a dot plot alignment of the re-sequenced Sr2 locus to itself which indicates the presence of several large near-identical duplications within this region.

PCR analysis using primers (Table 2) designed at the junctions of this 90 kb duplicated sequence amplified markers from the left junction with primers (Sr2_DupJn-F1×Sr2_DupJn-R1) and right junction with primers (Sr2_DupJn-F2×Sr2_DupJn-R2) junctions from all wheat lines tested, while the middle junction only amplified in Sr2 positive wheats with primers (Sr2_DupJn-F2×Sr2_DupJn-R1) including Yaroslav emmer, the tetraploid donor and the derived hexaploid wheat cv Hope (FIG. 2E). Susceptible wheats Marquis and Mace did not show amplification of this marker. These results indicate that the 90 kb duplication (SEQ ID NO: 71) is unique to Sr2 carrying wheats.

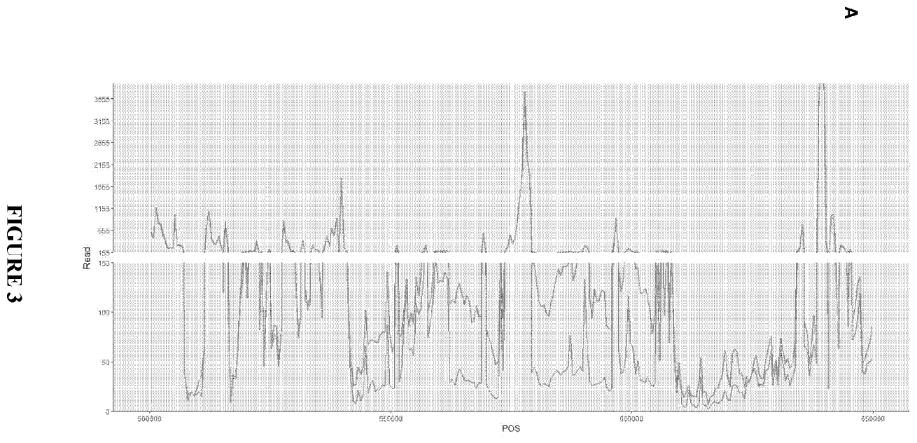

Relative copy number analysis of the TaPMP-B3 gene using digital droplet PCR that CS(Hope3B) contained 4.7 times higher copy number of this gene compared to Marquis (FIG. 2E). Analysis of Illumina read coverage across the locus in sequence data derived from the isolated 3B chromosomes of Marquis and Hope (De Oliveira et al 2020) also showed that the approximately 90 kb duplication region carrying the candidate genes had 4-5× higher read coverage in CS(Hope3B) compared to Marquis (FIG. 3). Together these data suggest that the resistant CS(Hope3B) contains either four or five copies of the duplicated region while Marquis contains a single copy.

TABLE 3
List of annotated genes and their positions at Sr2 locus.

| S No. | Gene | start | end | strand | Support |
|---|---|---|---|---|---|
| 1 | TaGLP3-3 | 917 | 1715 | − | BLAST/RNASeq |
| 2 | TaGLP3-4 | 2325 | 3137 | + | BLAST |
| 3 | TaGLP3-7 | 33129 | 33940 | + | BLAST/RNASeq |
| 4 | TaGLP3-8 | 40445 | 41254 | − | BLAST/RNASeq |
| 5 | TaGLP3-9 | 42011 | 42822 | + | BLAST/RNASeq |
| 6 | TPR1-1 | 108776 | 111940 | + | BLAST/RNASeq |
| 7 | D8LAL2-1 | 125154 | 127439 | − | BLAST/RNASeq |
| 8 | Unknown1-1 | 128103 | 128409 | − | FGNESH/RNASeq |
| 9 | CA687088-1 | 129235 | 129411 | − | BLAST/RNASeq |
| 10 | PMP3-1 | 149973 | 152111 | + | BLAST/RNASeq |
| 11 | TPR2-1 | 177291 | 178606 | + | BLAST/RNASeq |
| 12 | TPR1-2 | 198312 | 201476 | + | BLAST/RNASeq |
| 13 | D8LAL2-2 | 214685 | 216970 | − | BLAST/RNASeq |
| 14 | Unknown1-2 | 217634 | 217940 | − | FGNESH/RNASeq |
| 15 | CA687088-2 | 218766 | 218942 | − | BLAST/RNASeq |
| 16 | PMP3-2 | 239454 | 241592 | + | BLAST/RNASeq |
| 17 | TPR2-2 | 266794 | 268109 | + | BLAST/RNASeq |
| 18 | D8LAL1 | 278562 | 279656 | − | BLAST/RNASeq |
| 19 | CA696601-1 | 319647 | 320021 | − | BLAST/RNASeq |
| 20 | CN010964-1 | 341758 | 342905 | − | BLAST/RNASeq |
| 21 | CA694126 | 343768 | 344365 | − | BLAST/RNASeq |
| 22 | SPT19093-1 | 357830 | 364649 | − | BLAST |
| 23 | CA681074-1 | 374483 | 374812 | − | BLAST/RNASeq |
| 24 | CA681074-2 | 391779 | 392108 | − | BLAST/RNASeq |
| 25 | SPT19093-2 | 411262 | 418215 | − | BLAST |
| 26 | CA681074-3 | 428984 | 429313 | − | BLAST/RNASeq |
| 27 | FLA | 544500 | 545117 | + | FGNESH/BLAST |
| 28 | BEX1-1 | 564169 | 565601 | − | FGNESH/BLAST |
| 29 | BEX1-2 | 584119 | 585551 | − | FGNESH/BLAST |
| 30 | BEX2-1 | 592510 | 593765 | − | FGNESH/BLAST |
| 31 | BEX1-3 | 620292 | 621724 | − | FGNESH/BLAST |
| 32 | BEX2-2 | 628724 | 629979 | − | FGNESH/BLAST |
| 33 | BEX2-3 | 665291 | 666366 | − | FGNESH/BLAST |
| 34 | BM138011-1 | 682680 | 683033 | − | BLAST/RNASeq |
| 35 | DOX1 | 686491 | 690139 | + | FGNESH/BLAST |
| 36 | CA681101 | 696887 | 697285 | − | BLAST/RNASeq |
| 37 | BM138011-2 | 700402 | 700763 | − | BLAST/RNASeq |
| 38 | BTB | 730891 | 733026 | − | BLAST/RNASeq |
| 39 | RGA1 | 734381 | 740236 | + | FGNESH/BLAST |

Genes in black are placed between the recombination breakpoints. Genes with multiple copies are represented with a −number.

TABLE 4
Sequence analysis of Sr2 locus in CS(Hope3B) showing
the positions of duplications of 15 kb and above.

| | Positions | Statistics | Mutations |
|---|---|---|---|
| # | Copy 1 | Copy2 | size |
| 1 | 96713-186226 | 186251-275723 | 89514/89473 |
| 2 | 346063-379114 | 399509-433592 | 33052/34084 |
| 3 | 477804-509114 | 493643-524952 | 31311/31310 |
| 4 | 583295-611599 | 619460-647797 | 28305/28338 |
| 5 | 567757-587753 | 547757-567796 | 19997/20040 |
| 6 | 379113-399514 | 416266-436698 | 20402/20433 |
| 7 | 509180-524952 | 477804-493577 | 15773/15774 |
| 8 | 379113-396386 | 362837-379114 | 17274/16278 |

### Example 4: Gene Expression and Identifying Candidate Genes for Sr2 Resistance

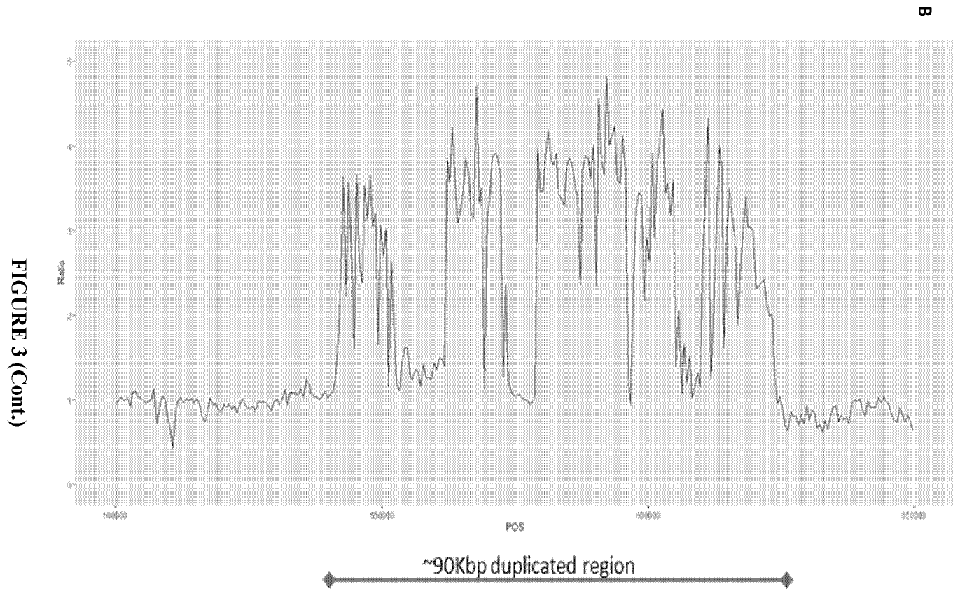

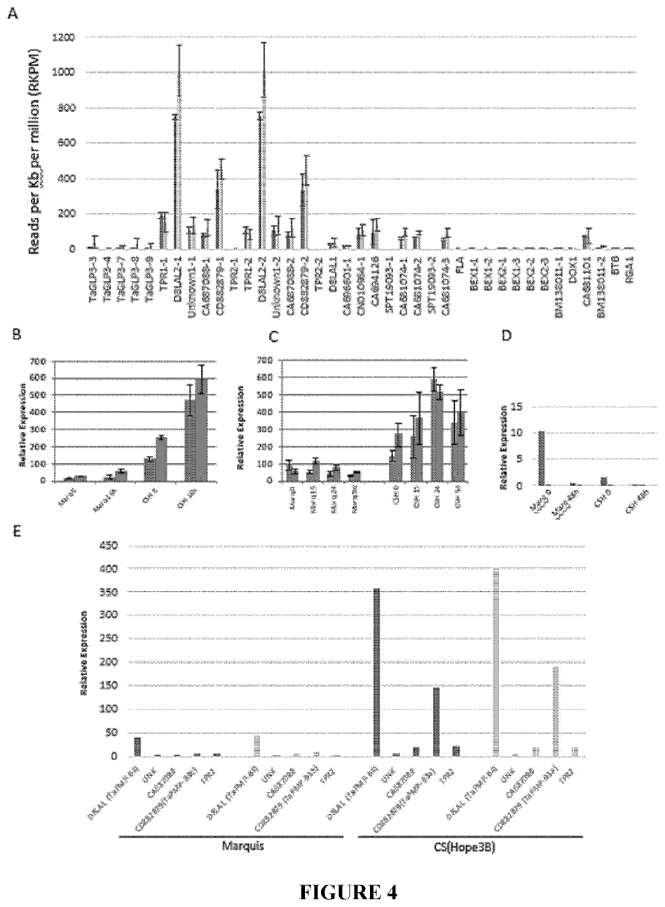

The expression of genes at the Sr2 locus was analysed using RNAseq. FIG. 4A shows the expression of the annotated genes across the Sr2 locus. Of the six annotated genes located on the 90 kb duplication, two predicted genes namely D8LAL2 and CD882879, stood out as the most highly expressed in both powdery mildew infected leaves and after infection with stem rust in resistant CS(Hope3B).

The expression these two genes in CS(Hope3B) and the susceptible wheat cv Marquis was assessed by quantitative reverse transcription PCR (qRT-PCR). The expression of D8LAL2 and CD882879 was about 10× higher in leaf sheaths of CS(Hope3B) compared to cv Marquis and induced by up to 2-fold and 4-fold respectively in CS(Hope3B) during infection by stem rust (FIG. 4B). The expression of these genes was also higher in leaves of CS(Hope3B) compared with Marquis after infection with powdery mildew (FIG. 4C). There was very low or no expression of these genes at seedling stage consistent with the resistance response occurring only in adult plant stage (FIG. 4D). FIG. 4E shows the gene expression using RT-PCR of the six genes present on the 90 kb duplicated region in flag-1 leaf collected at 0 and 48 hours post infection with stem rust in CS(Hope3B) and Marquis. The RT-PCR results were consistent with data from RNAseq experiment showing strong expression of D8LAL2 and CD882879 in CS(Hope3B) while expression in Marquis was generally low.

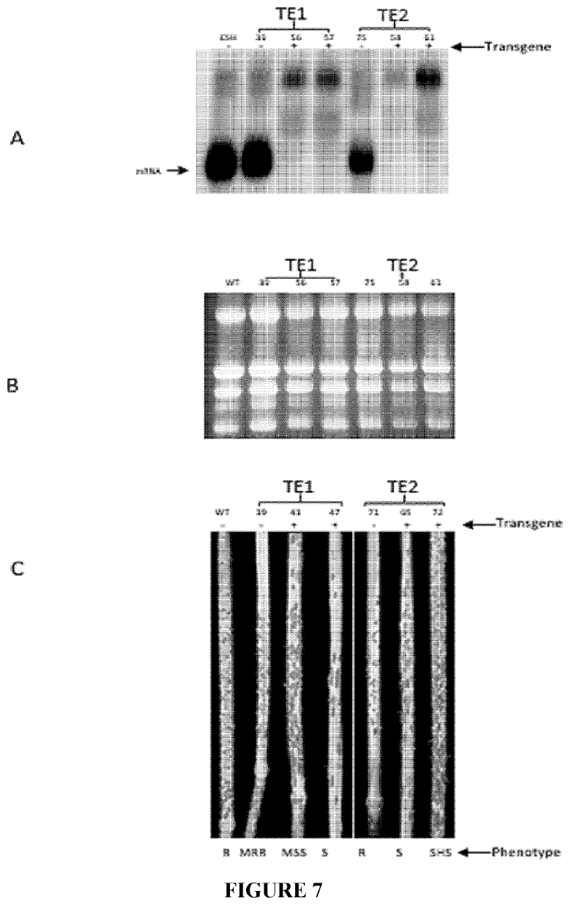

While no sequence polymorphism was identified in D8LAL2 between Marquis and CS(Hope3B), a single SNP was identified in CD882879 that resulted in predicted amino acid change (D2V) FIG. 5A. D8LAL2 and CD882879 were predicted to encode proteins containing 406 and 374 amino acids, respectively. The TMHMM program predicted both D8LAL2 and CD882879 to be transmembrane proteins and identified 10 putative transmembrane domains in CD882879 (FIG. 5B) and 7 in D8LAL2 (FIG. 5C).

The structures of TaPMP3a and TaPMP4 were predicted using Alphafold2 and both proteins were predicted to contain eleven helices arranged in parallel, consistent with these being transmembrane helices (FIG. 5D). All of the predicted TM regions (FIG. 5C) corresponded to one of the predicted alpha helix structures, while the additional helices in each protein all corresponded to regions with weak TM predictions. These data are consistent with both the TaPMP3a and TaPMP4 genes encoding integral membrane proteins with eleven transmembrane helices. A search of the protein structure database using Dali did not detect any known protein structures with significant similarity to the predicted structures of TaPMP-B3a or TaPMP-B4.

To summarize the key features of the two haplotypes: CS(Hope3B) carried duplicated copies of D8LAL2 and CD882879 with one predicted amino acid change in CD882879 compared to the Marquis allele. The Marquis haplotype contained only a single copy of both genes as seen by lack of duplication of this region (FIG. 2E). The expression level of both genes was higher in CS(Hope3B) compared to Marquis and induced by infection with stem rust and powdery mildew. Based on results from fine mapping (FIG. 1), the expression analysis (FIG. 4) and predicted amino acid change (FIG. 5A), D8LAL2 and CD882879 were thus considered strong candidates for Sr2 resistance. It was decided to rename the Hope allele of CD882879 as Putative Membrane Protein gene 3 (TaPMP-B3a), the Marquis allele TaPMP-B3b and D8LAL2 as TaPMP-B4a.

FIG. 5D shows a schematic organization of genes TaPMP-B3 and TaPMP-B4 on the chromosome. The genes are separated by 22,533 bp of intervening sequence. Additionally, the resistant parent CS(Hope3B) carries 2 copies of both of these genes owing to the 90 kb duplication of the region, the comparator susceptible variety compared to Marquis does not have this duplication.

### Example 5: Functional Proof: RNAi Silencing Shows that TaPMP3A is Required for Sr2 Resistance

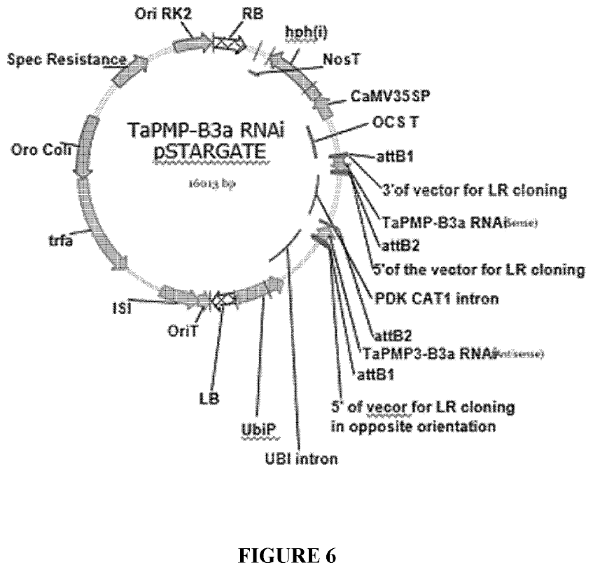

The inventors transformed CS(Hope3B) with a hpRNA construct (FIG. 6) designed to silence TaPMP-B3 and generated TO transgenic lines from two independent events. Total RNA was prepared from flag-1 leaf at 48 hours post infection. RNA gel blot analysis showed that TaPMP-B3 expression was undetectable in T1 progeny of these lines containing the silencing construct, while strong expression was observed in null segregants and non-transgenic controls (FIG. 7A). Equal amounts of RNA was run in agarose gel to confirm approximately even loading of RNA for Northern blot analysis (FIG. 7B). T1 progeny of the same transgenic events carrying the TaPMP-B3 hpRNA construct were assayed for response to stem rust. T1 plants containing the silencing transgene were moderately to highly susceptible while null segregants and non-transgenic controls were resistant (FIG. 7C).

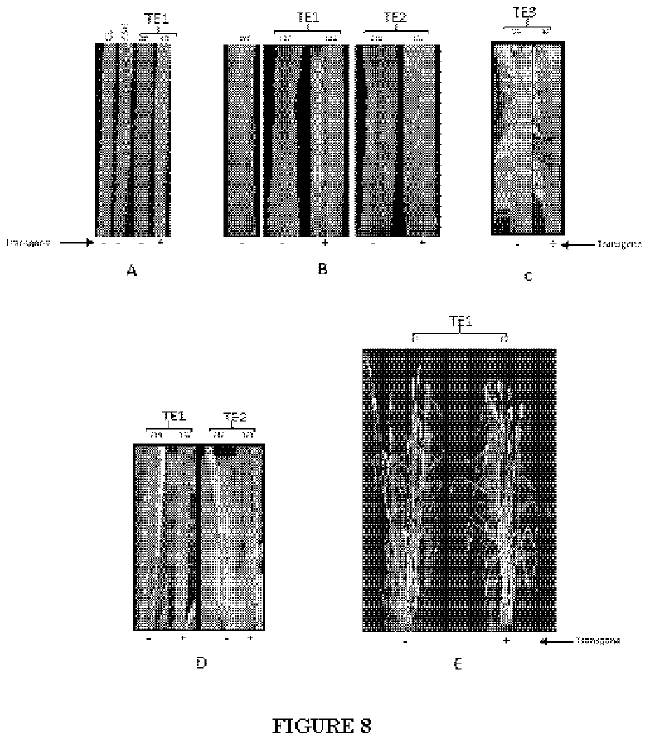

Sr2 is also linked to resistance against leaf rust (Lr27) and powdery mildew (Pm) (Mago et al., 2014). The inventors therefore evaluated the plants from same transgenic events for response to leaf rust and powdery mildew. T1 plants carrying the silencing construct showed loss of leaf rust resistance conferred by Lr27 and T2 plants carrying the PMP-B3a-hp construct were susceptible to powdery mildew (FIGS. 8A and 8B). Thus, TaPMP-B3a is required for Sr2-mediated resistance and resistance against leaf rust and powdery mildew.

Previous work by Tabe et al. (2019) reported an association of Sr2 resistance with leaf necrosis induced by application of petroleum jelly or heat shock. To determine if transcriptional silencing of TaPMP-B3a can lead to loss of abiotic phenotypes associated with Sr2, we applied Vaseline to a small section of leaf from plants of T2 families from two transgenic events. Plants that carried the PMP-B3a-hp construct showed no necrosis compared to the non-transgenic sib lines (FIG. 8C). T2 progeny that carried the transgene also lacked heat shock induced leaf necrosis at the seedling stage (FIG. 8D). Transgenic plants carrying PMP-B3-hp also showed a lack of leaf senescence/necrosis at anthesis (FIG. 8E). In addition, PMP-B3a-hp transgenics failed to develop pseudo black chaff (PBC), which was previously associated with cell death in photosynthetic cells in stem and ear. Based on these results, TaPMP-B3a is not only required for resistance to three fungal pathogens but is also required for the expression of abiotic stress phenotypes.

### Example 6: Functional Proof—Gene Complementation with TaPMP-B3

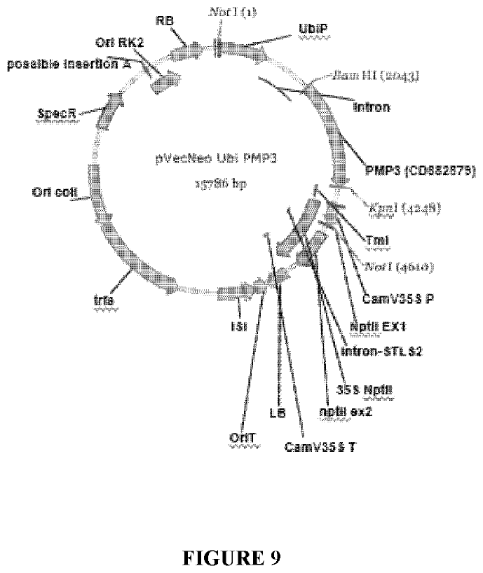

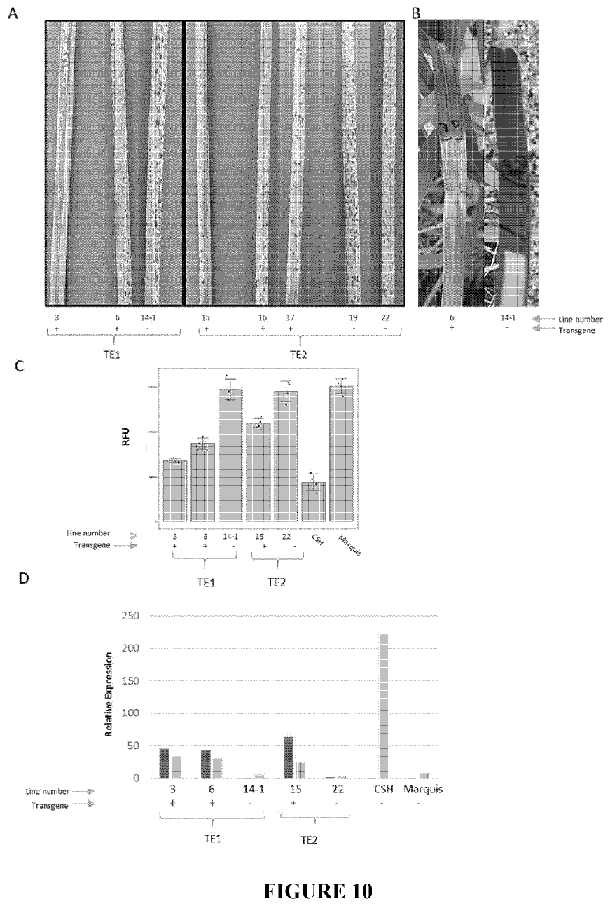

Silencing TaPMP-B3 by RNAi indicated that this gene was required for Sr2-mediated fungal resistance. To confirm the role of TaPMP-B3a in conferring a resistance response, we transformed the susceptible wheat cv Marquis with the TaPMP-B3a gene which was isolated from CS(Hope3B) and expressed under Maize ubiquitin promoter (FIG. 9). T1 families from 5 independent transgenic events were phenotyped for stem rust resistance and leaf necrosis. Plants carrying the transgene were resistant and showed fewer and smaller rust pustules and necrotic lesions on leaf sheaths than non-transgenic sib lines (FIG. 10A). Flag leaves of transgenics that were treated with petroleum jelly developed necrotic lesions which were not present in null segregants (FIG. 10B). Chitin quantification showed that the amount of fungal biomass in infected sheaths of the transgenic lines was less than in null sib lines or the susceptible Marquis line, although still higher than in CSH (FIG. 10C). Statistical analysis using T-test (Table 5) showed that the fungal biomass in lines carrying the transgene was significantly less in plants carrying the transgene, in comparison the null segregants or the susceptible parent Marquis.

Under the constitutive ubi promoter, expression of the TaPMP-B3 gene was at least 10 times higher in transgenic lines than in the nulls or the wildtype Marquis control in both uninfected and infected plants, while the endogenous gene in CSH was strongly induced during infection and expressed at much higher level than in transgenics (FIG. 10D). No PBC was observed in any of the resistant transgenic plants indicating that the increase in expression of TaPMP-B3a was not sufficient for PBC development in the Marquis background. Based on these results the expression of TaPMP-B3a gene was sufficient to confer stem rust resistance in the Marquis background. This does not rule out that TaPMP-B4 is also required for resistance since Marquis contains an copy of the TaPMP-B4 gene, albeit expressed at low level. Marquis lacks the complementary Lr31 gene on chromosome 4B, so the inventors did not test the response to leaf rust in these transgenics.

To summarize the functional analysis, TaPMP-B3a is required for stem rust resistance based on transcriptional silencing of the gene in CS(Hope3B) (Example 5) and overexpression of TaPMP-B3a in the Marquis background (contains Ta-PMP4) is sufficient to confer resistance (Example 6). In addition, silencing TaPMP-B3a resulted in loss of leaf rust resistance conferred by Lr27/Lr31, loss of powdery mildew resistance and loss of the hypoxia-induced necrotic phenotype associated with Sr2 (Example 5). It was also demonstrated that overexpressing TaPMP-B3a induced the hypoxia-induced necrotic phenotype in Marquis plants (Example 6).

TABLE 5
P-values showing the level of significance of fungal biomass
between the Marquis trangenics carrying TaPMP-B3a when compared
to each other and the resistant and susceptible parents.

| Line No. | 3 | 6 | 14 | 15 | 22 | CSH | Marquis |
|---|---|---|---|---|---|---|---|
| 3 | | | | | | | |
| 6 | 0.0054317 | | | | | | |
| 14 | 0.0005787 | 0.0003657 | | | | | |
| 15 | 0.0001511 | 0.001522 | 0.003378425 | | | | |
| 22 | 0.0005916 | 0.0003848 | 0.818799492 | 0.003877 | | | |
| CSH | 0.0128439 | 0.0005501 | 1.13E−05 | 8.91E−05 | 1.16E−05 | | |
| Marquis | 0.0001181 | 2.77E−05 | 0.622660463 | 0.0003402 | 0.4558751 | 3.23E−06 | |

### Example 7: Functional Proof—RNAi Silencing and Gene Complementation Shows that TaPMP-B4 is Required for Sr2 Resistance

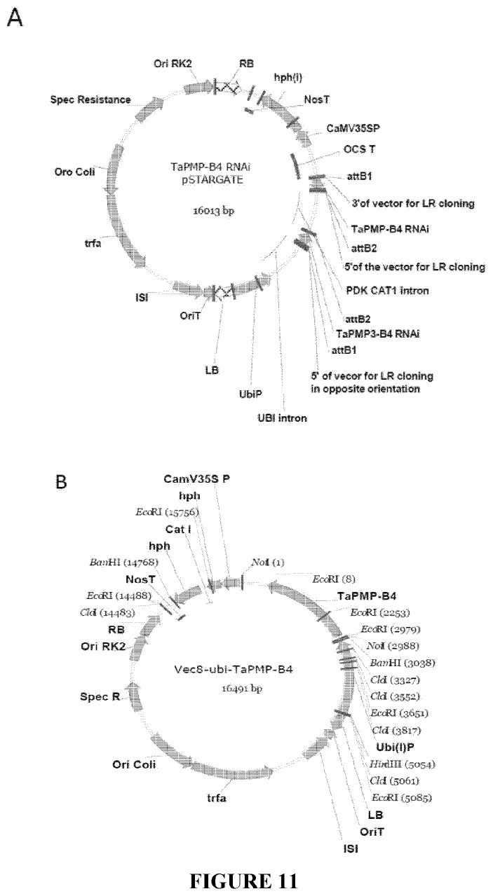

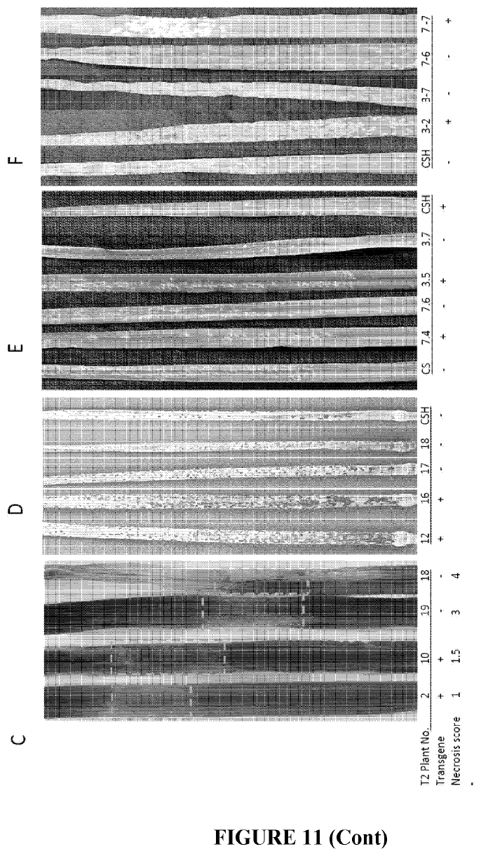

A construct (FIG. 11A) designed to silence TaPMP-B4 in the resistant parent CS(Hope3B) was generated. T2 transgenic lines from two independent events were analysed (FIG. 11C). Table 6 shows the Vaseline induced necrotic phenotypes of plants from T2 families. T2 plants carrying the hairpin construct showed loss of necrosis in several plants compared to the null lines which showed strong necrosis associated with Sr2. No necrosis was seen in Marquis leaves treated with Vaseline. The inventors also showed that T2 plants carrying the RNAi construct became more susceptible to stem rust, leaf rust and powdery mildew as compared to null segregants which lacked the transgene (FIG. 11D, 11E, 11F)

A transgenic Marquis line homozygous for the TaPMP-B3a transgene under maize Ubiquitin promoter was super-transformed with Vec8-Ubi-TaPMP-B4 (FIG. 11B). Gene expression analysis showed that expression of Ubi-TaPMP-B4 was 20× higher in transgenics when compared to marquis. All plants carrying the transgene were resistant to the stem rust pathotype pgt21-0. There was no difference in resistance phenotype of lines carrying TaPMP-3a alone or both TaPMP-3a and TaPMP-B4.

It has therefore been demonstrated that the expression of Sr2 associated rust and powdery mildew resistance and leaf necrosis involves both TaPMP-B3a and TaPMP-B4. Thus, TaPMP-B3a and TaPMP-B4 share important characteristics: 1. The expression level of both genes is higher in the resistant haplotype and the expression of both genes was induced upon infection with stem rust and powdery mildew. 2. They are located on the duplicated region which was only detected in Sr2 resistant germplasm. 3. RNAi silencing of either gene results in loss of Vaseline induced leaf necrosis as well as rust and powdery mildew resistance. Furthermore, the resistant CS(Hope3B) accumulated much less fungal biomass than resistant transgenic Marquis which was transformed with TaPMP-B3a suggesting that the increased expression of an additional gene or genes (eg. TaPMP-B4) contributes to reducing fungal growth in CS(Hope3B). Thus the increased expression of duplicated copies of both TaPMP-B3a and TaPMP-B4a genes are involved in conferring full Sr2-mediated stem rust resistance in CS(Hope3B). These genes and their variants are useful individually and together for providing new fungal resistance genetic resources for deployment in agricultural crops.

TABLE 6
Analysis of T2 plants carrying the TaPMP-B4 hairpin construct for
loss of Vaseline induced necrosis associated with Sr2 resistance.

| | | T2 | Vaseline |
|---|---|---|---|
| | Transgene | Progeny | Necrosis |
| T1- Name | (T1 plant) | number | score |
| PC174-3-2 | + | 1 | 0.5 |
| | | 2 | 0 |
| | | 3 | 0 |
| | | 4 | 2.5 |
| | | 5 | 2 |
| | | 6 | 4 |
| | | 7 | 0 |
| | | 8 | 4 |
| PC174-3-5 | + | 9 | 0 |
| | | 10 | 0 |
| | | 11 | 0 |
| | | 12 | 0 |
| | | 13 | 0 |
| | | 14 | 0 |
| | | 15 | 0 |
| | | 16 | 0 |
| PC174-3-7 | − | 17 | 4 |
| | | 18 | 4 |
| | | 19 | 4 |
| | | 20 | 4 |
| PC212-7-4 | + | 21 | 0.5 |
| | | 22 | 0.5 |
| | | 23 | 0.5 |
| | | 24 | 0 |
| | | 25 | 0 |
| | | 26 | 0.5 |
| | | 27 | 1 |
| | | 28 | 0 |
| PC212-7-6 | − | 29 | 3 |
| | | 30 | 4 |
| | | 31 | 3 |
| | | 32 | 4 |
| PC212-7-7 | + | 33 | 0 |
| | | 34 | 1 |
| | | 35 | 0 |
| | | 36 | 2 |
| | | 37 | 0.5 |
| | | 38 | 1 |
| | | 39 | 3 |
| | | 40 | 0.5 |
| Marquis | WT-S | 41 | 0 |
| Marquis | WT-S | 42 | 0 |
| Marquis | WT-S | 43 | 0 |
| Marquis | WT-S | 44 | 0 |
| CS(Hope3B) | WT-R | 45 | 4 |
| CS(Hope3B) | WT-R | 46 | 4 |
| CS(Hope3B) | WT-R | 47 | 4 |
| CS(Hope3B) | WT-R | 48 | 4 |

WT-S = Wildtype Susceptible parent
WT-R = Wildtype Resistant parent

### Example 8: Functional Proof—Transient Expression of TaPMP-B3 and TaPMP-B4 Induces Cell Death in Nicotiana Spp

Expression of TaPMP-B3a and TaPMP-B4 from the Sr2 locus in wheat is associated with cell death phenotypes including pseudo black chaff and hypoxia and heat stress induced necrosis. To determine whether these genes can induce similar responses in other plants, the inventors carried out transient expression assays in planta as described in Example 1.

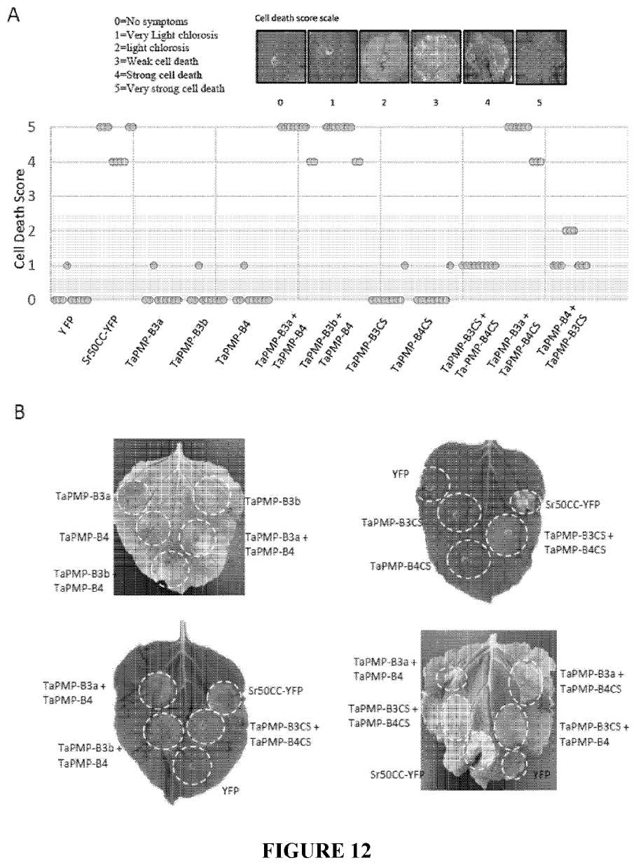

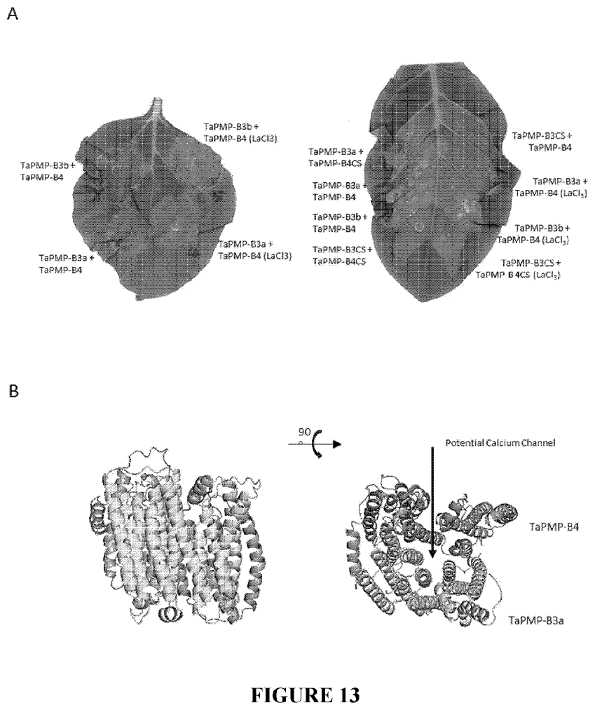

Five constructs were generated representing the variants of TaPMP-B3 and TaPMP-B4 and expressed alone or in pairwise combinations in N. benthiamiana and N. tabacum (FIGS. 12 and 13Aa) via Agrobacterium tumefaciens (strain GV3101) transient transformation. Referring to FIGS. 12A and B it is shown that the expression of any of the TaPMP-B3 or TaPMP-B4 variants alone did not produce a visible cell death phenotype. However, co-expression of TaPMP-B3a or TaPMP-B3b with TaPMP-B4 resulted in a strong cell death phenotype. This indicates that the two proteins can act together to induce necrosis and that the single amino acid difference between TaPMP-B3a and TaPMP-B3b does not affect this function.

Also the inventors concluded that the TaPMP-B3b protein variant encoded by the Marquis genotype is capable of conferring functional resistance if expressed at sufficient level and that the phenotypic difference between the Hope and Marquis genotypes is dependent on the high expression conferred by the tandem duplication in the Hope haplotype. Co-expression of TaPMP-B4CS (the allelic variant from Chinese Spring) with either TaPMP-B3a or TaPMP-B3b also resulted in strong cell death (FIG. 12), suggesting that this protein is also fully functional in conferring the cell death phenotype. However, co-expression of TaPMP-B3CS with either TaPMP-B4 or TaPMP-B4CS gave a weaker cell death phenotype suggesting that this protein has a weaker resistance-inducing activity.

These data confirm that the two proteins TaPMP-B3 and TaPMP-B4 act together to confer cell death-related phenotypes. Because these proteins are predicted as intrinsic membrane proteins, they may act as ion channels. Treatment of leaf tissue with the Calcium channel inhibitor LaCl3 resulted in strong suppression of the cell death phenotype caused by co-expression of TaPMP-B3a or TaPMP-B3b with TaPMP-B4 (FIG. 13A). This result suggests that the proteins may interact to form a functioning calcium ion channel. A structural interaction prediction by AlphaFold v2.1.1 multimer suggested that the two proteins could interact to form a dimer containing a central pore, which could act as an ion channel (FIG. 13B). This structural prediction is consistent with the requirement for co-expression of both proteins to confer resistance.

### Example 9: Mutagenesis of CS(Hope3B) to Isolate Loss of Function Mutants for Stem Rust Resistance

The inventors performed EMS mutagenesis as described by Mago et al. (2017) by treating CS(Hope3B) seed with 0.4% EMS. From approximately 10,000 M1 single heads were harvested and screened for APR response to Australian stem rust race 98-1,2,3,5,6 in the field. From the rust screening of M2 families, several susceptible mutants were identified. These mutants were subsequently confirmed in M3 and M4 generations in the field and, in a climate-controlled growth cabinet. Analysis of the mutants using markers developed from Hope3B Sr2 region showed that all the mutants had large deletion of the 3B chromosome. The lack of mutants carrying point mutations/small deletions was consistent with the hypothesis that more than one gene contributed to Sr2 resistance because it is highly unlikely that random mutagenesis would generate independent mutations within multiple gene copies.

### Example 10: Development of Diagnostic Marker for Sr2 Based on the Junction Between the Duplicated Region

The inventors identified the junctions of the 90 kb duplication and designed primers across one of the junctions that amplify a unique product to verify the presence of the duplicated region. FIG. 2E shows the amplification of the middle junction marker in the resistant parent CS(Hope3B), the original Sr2 donor Yaroslav emmer, cv Hope, and susceptible parent Marquis using primers Sr2_DupJn-F2 and Sr2_DupJn-R1 (Table 2). The marker was converted into a high throughput KASP marker and tested across a wide range of germplasm with known Sr2 status (Table 7). The results showed that the 90 kb duplication was unique to Sr2 carrying wheats. Two critical wheat lines cv Timgalen and Sinvalocho which are known to carry Lr27 but not Sr2 lacked the duplication junction and therefore possibly contain a single copy of PMP-B3b and PMP-B4a. Thus, it is likely that single copies of these genes, single copy of the gene pair, or other genes in the region, are sufficient to confer leaf rust resistance in these backgrounds. Primers sequences for the new marker are listed in Table 2.

The inventors have shown how to correctly identify materials that are carrying Sr2 resistance genotype. Variant sequences for correctly identifying resistant lines may be cloned using the primers described herein or designed using software-based tools commonly available.

TABLE 7
Allele survey of diverse wheat germplasm with known (or
presumed) Sr2 status using CAPS marker csSr2, SNP marker
from TaPMP-B3a and duplication specific marker.

| | | | | Duplication | |
|---|---|---|---|---|---|
| | | | TaPMP-B3a | PMP 3b- | |
| | Sr2 | | SNP | Dup.Inf × | Sr2 |
| | resistance | csSr2 | CD882879_F2 | PMP3b- | Based on |
| Wheat line | (Expected) | SNP | (KASP) | DupinR1 | Markers |
| Angas | − | n | T | − | − |
| Aroona | − | n | T | − | − |
| Arrino | − | n | T | − | − |
| Avocet | − | n | T | − | − |
| Banks | − | n | T | − | − |
| Bindawarra | − | G | T | − | − |
| Braewood | − | n | T | − | − |
| Cadoux | − | n | T | − | − |
| Camm | − | n | T | − | − |
| Chara | − | n | T | − | − |
| Chinese Spring | − | n | T | − | − |
| Federation | − | n | T | − | − |
| Frame | − | n | T | − | − |
| Gabo | − | n | T | − | − |
| Glenlea | − | n | T | | − |
| H45 | − | n | T | − | − |
| Halberd | − | n | T | − | − |
| Janz | − | n | T | − | − |
| Kelalac | − | n | T | − | − |
| Langdon | − | n | T | − | − |
| Marquis | − | G | T | − | − |
| Molineux | − | n | T | − | − |
| Opata | − | G | T | | − |
| Sunvale | − | n | T | − | − |
| Steinwendel | − | n | T | − | − |
| Tasman | − | n | T | − | − |
| Thatcher | − | G | T | − | − |
| Timgalen | − | G | T | − | − |
| Tincurrin | − | n | T | − | − |
| Trident | − | n | T | − | − |
| Westonia | − | n | T | − | − |
| Wyalkatchem | − | n | T | − | − |
| Yaralinka | − | n | | − | − |
| Yitpi | − | n | | − | − |
| Arthur | + | A | A | + | + |
| Baxter | + | A | A | + | + |
| Bluebird | + | A | A | + | + |
| Carnamah | + | A | A | + | + |
| Ciano67 | + | A | A | + | + |
| Cranbrook | + | A | A | + | + |
| Diamondbird | + | A | A | + | + |
| Dollarbird | + | A | A | + | + |
| Drysdale | + | A | A | + | + |
| Ega-Wiley | + | A | A | + | + |
| Eradu | + | A | | + | + |
| Gatcher | + | A | A | + | + |
| Hartog | + | A | A | + | + |
| Hope | + | A | A | + | + |
| Houtman | + | A | A | + | + |
| Khapstein | + | A | A | + | + |
| Kingbird | + | A | A | + | + |
| Kiritati | + | A | A | + | + |
| Lancer | + | A | A | + | + |
| Lerma Rojo | + | A | A | + | + |
| Lowan | + | A | A | + | + |
| Machete | + | A | A | + | + |
| Madden | + | A | A | + | + |
| Nuri70 | + | A | A | + | + |
| Ottawa | + | A | A | + | + |
| Parula | + | A | A | + | + |
| Pavon76 | + | A | A | + | + |
| Pelsart | + | A | A | + | + |
| Pembina | + | A | A | + | + |
| Redman | + | A | A | | + |
| Renown | + | A | A | | + |
| Rowan | + | A | A | + | + |
| Scout | + | A | A | | + |
| Selkirk | + | A | A | | + |
| Sonalika | + | A | A | + | + |
| Suneca | + | A | A | + | + |
| Sunstate | + | A | A | + | + |
| Ventura | + | A | | + | + |
| Kenya Page | + | A | A | + | + |
| Khapli -Ctlr4013 | + | A | A | + | + |
| Yaroslav emmer | + | A | A | + | + |
| Derrimut | (+)** | G | T | − | − |
| Ellison | (+)** | G | T | − | − |
| Kenya Plume | (+)** | G | T | − | − |
| Kukri | (+)** | G | T | − | − |
| Leichardt | (+)** | n | T | | − |
| Mace | (+)** | G | T | − | − |
| Pastor | (+)** | G | T | − | − |
| Sceptor | (+)** | G | T | − | − |
| Siete-Cerros | (+)** | G | T | − | − |

n = No amplification
(+) = Sr2 based on PBC
**Presumed to carry Sr2

### Example 11: Enhanced Fungal Resistance by Expression of TaPMP-B3a and TaPMP-B4 in Other Plant Species

Sorghum transformation was undertaken to evaluate the resistance conferred by TaPMP-B3 and TaPMP-B4 in sorghum using the wheat sequences PMP-B3 and PMP-B4, as well as the sorghum TaPMP-B3 homolog A0A1B6Q3F (SEQ ID NO: 25) and TaPMP-B4 homolog C5XJV5 (SEQ ID NO: 33). Methods for sorghum transformation are described in the art for example see WO2016/104583 or WO2017/210719.

Plants of grain sorghum inbred line Tx-430 may be grown in a plant growth chamber (Conviron, PGC-20 flex) at 28±1/20±1° C. (day/night) temperature, with a 16 h photoperiod of 600 μmol/s m2 in Canberra, ACT, Australia. Panicles can be covered with white translucent paper bags before flowering. Immature embryos are harvested from panicles 12-15 days after anthesis. After sterilising the immature seeds as described by Liu and Godwin (2012) the immature embryo explants from 1.4 mm to 2.5 mm in length are aseptically isolated from sorghum plants and transformed with Agrobacterium strain carrying vectors assembled as described in Example 1 to generate transgenic sorghum plants. Agrobacterium transformation methods are performed as previously described by Che et al (2018). The integrated copy number of the T-DNA can be determined using PCR as described in Example 1. Independent transgenic plants are identified which were transformed with the wheat or sorghum Sr2 homolog transgene. All transgenic plants and wild-type control plants which had been subjected to the tissue culture steps involved in transformation but lacking the Sr2 transgene can be infected with Puccinia purpurea. Rust sporulation is expected to be observed on leaves of the control plants but not on the positive Sr2 transgenic plants of the TO generation.

Experiments are also carried out to modify the genes encoding the orthologous pairs in Example 12 identified as homologs of Sr2, to increase the expression in planta of the polypeptides to create resistant plants. When the T1 generation of plants are tested, all of the plants containing Sr2 genes showed the leaf rust and/or mildew resistance phenotype when tested on seedlings, whereas all of the plants lacking the Sr2 genes showed fungal susceptibility. These results confirmed that the isolated Sr2 genes and orthologues are functionally active and sufficient to confer leaf rust resistance in other crops. Providing resistance genes for these plant species and other plants.

### Example 12: Enhanced Fungal Resistance by Increasing Expression of Orthologous Gene Pairs in Monocot Crop Species

At the Sr2 locus TaPMP-B3 and TaPMP-B4 are arranged in ‘head to head’ configuration separated by approx. 23 kb of DNA sequence (FIG. 5D). In RNAseq and qRT-PCR experiments we have shown that the expression of both genes was increased indicating that these genes share a common regulatory sequence. The amino acid sequences of TaPMP-B3 and TaPMP-B4 were used to search for related predicted membrane proteins in the EnsemblPlants database and identified corresponding genes that were also arranged ‘head to head’ and were located within close proximity of each other (within 30 kb). The exception was a gene pair on chromosome 1 of rice which was arranged ‘head to tail’ and the gene pair on chromosome 3 of maize which was separated by approx. 265 kb (Table 8).

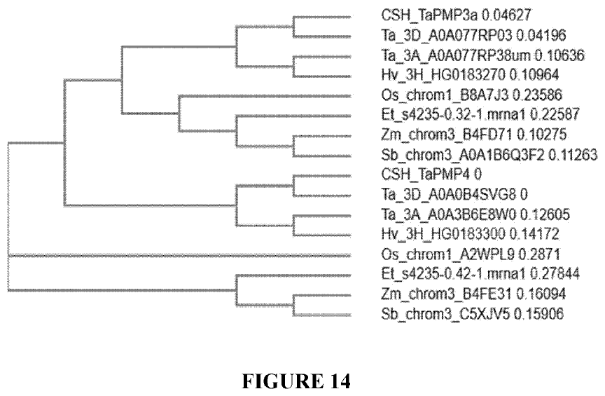

Given the known evolutionary relationships between monocot genomes, it is predicted that gene pairs in syntenic regions of wheat group 3, barley 3H, rice chromosome 1, sorghum chromosome 3, maize chromosome 3 and the unanchored scaffold in tef share common ancestral origin and function. FIG. 14 shows the phylogenetic relationship of orthologous protein sequences for TaPMP3 and TaPMP4.

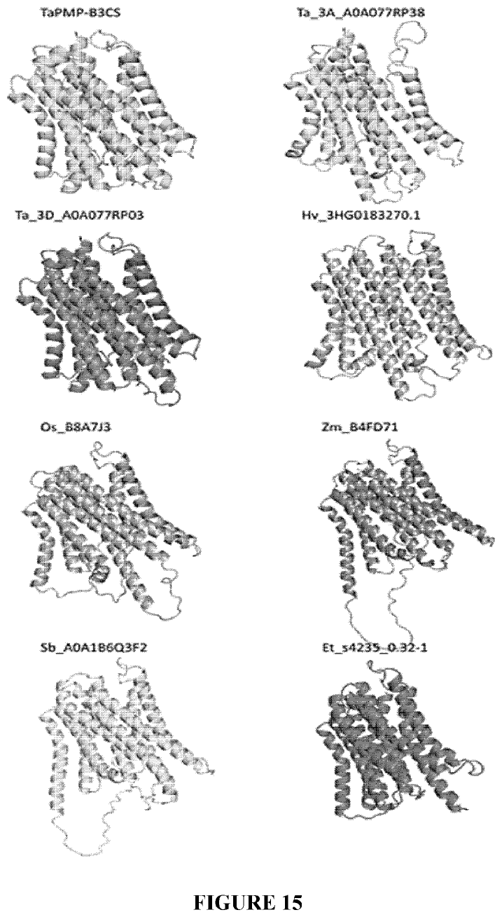

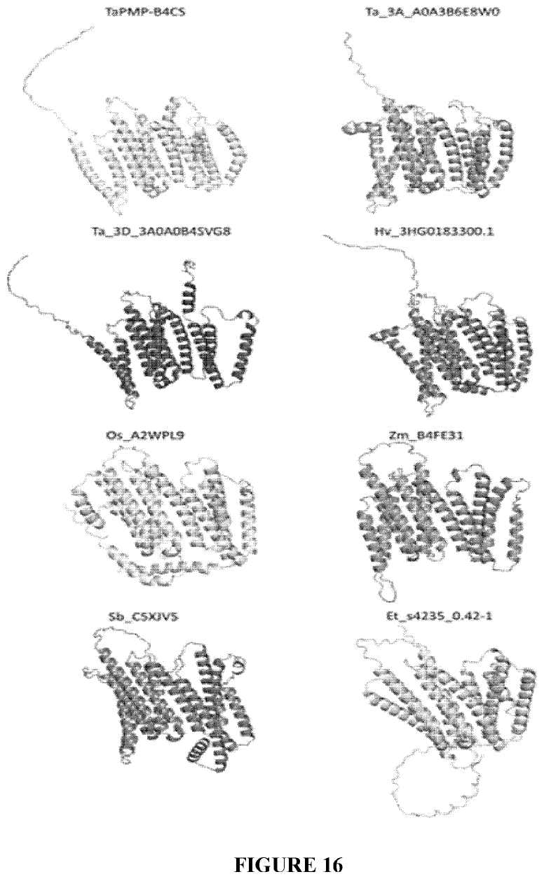

The structures of these homologs were predicted using alphafold2.1.1 and all showed very similar structures to those predicted for Ta-PMPB3a and TaPMP4 centred on an 11-helix bundle (FIGS. 15, 16 and Table 8). Structural comparisons using Dali showed very high Z-scores (>20 indicates two structures are definitely homologous, between 8 and 20 means the two are probably homologous) and low RMSD values with a high proportion of structurally equivalent residues, indicating that these are highly similar structures (Table 9).

In the predicted dimer structure of TaPMP-B3a (wheat cv Chinese Spring, SEQ ID NO:85) and TaPMP-B4 (wheat cv Chinese Spring, SEQ ID NO:86) there is extensive contact between transmembrane helices of the two proteins. These include interactions involving contact of the α1 and α2 helices of TaPMP-B3a with α3, α6 and α7 of TaPMP-B4, and also α9 and α11 of TaPMP-B3a with α8 of TaPMP-B4. These largely involve interactions between exposed hydrophobic surfaces, but also include specific polar contacts between residues R9 (α1 of TaPMP-B3a) with E204 (α7 of TaPMP-B4), E13 (α1 of TaPMP-B3a) with K208 (α7 of TaPMP-B4), Q68 (α2 of TaPMP-B3a) with R195 (α6-7 loop of TaPMP-B4), and E269 (α8-9 loop of TaPMP-B3a) with R251 (α8 of TaPMP-B4). There is also a polar contact between T117 (α4 of TaPMP-B3a) with W3 in the extended N-terminus of TaPMP-B4 (which adopts an alpha-helical fold in the dimer while being unstructured in the monomer predictions). The potential channel between the two proteins is formed by the alpha helices α1, α5, α9 of TaPMP-B3a and α7 and α8 of TaPMP-B4. Thus, residues in these regions involved in the interaction between the two proteins and in the formation of the potential channel are likely to be important for the function of these proteins in conferring pathogen resistance. The amino acid coordinates of the alpha helices involved in interaction and pore formation are: Ta PMP-B3a, α1, 1-28, α2, 44-69, α4, 100-126, α5, 134-165, α9, 267-295, α11, 338-374; and TaPMP-B4, α1, 1-16, α3, 93-123, α6, 171-194, α7, 198-226, α8, 229-252 (data not shown).

The present application claims priority from AU2020904574 filed 9 Dec. 2020, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

TABLE 8
Predicted proteins of gene pairs identified in monocot crop species by BlastP searches
with TaPMP-B3 and TaPMP-B4 queries. Corresponding gene pairs are located in close proximity
and arranged ‘head to head’ with the exception of rice chromosome 1 gene pair
(see text). Amino acid sequence identity and number of predicted transmembrane helices
are shown for TaPMP-B3 (left) and TaPMP-B4 (right) homologs, respectively.

| | | | | | Number of. | Number of. |
|---|---|---|---|---|---|---|
| | | | | | transmembrane | transmembrane |
| | | | | | helices | helices |
| | | | | Amino | predicted for | predicted for |
| | | | | acid % id | TaPMP-B3a/ | TaPMP-B3a/ |
| | | | | compared to | TaPMP-B4 | TaPMP-B4 |
| | | Protein pair | | TaPMP-B3a/ | homologs | homologs |
| | chromosome | TaPmp3 query | TaPmp4 query | TaPMP-B4 | by TMHMM | by TMHMM |
| Triticum | 3B | TaPMP-B3a | TaPMP-B4 | | 11/11 | 10/7 |
| aestivum | | | | | | |
| (Chinese | | | | | | |
| Spring) | | | | | | |
| | 3A | A0A077RP38 | A0A3B6E8W0 | 60/62 | 10/11 | 8/6 |
| | 3D | A0A077RP03 | A0A0B4SVG8 | 90/100 | 11/10 | 9/7 |
| Hordeum | 3H | 3HG0183270.1 | 3HG0183300.1 | 58/63 | 10/11 | 11/7 |
| vulgare | | | | | | |
| (Golden | | | | | | |
| Promise) | | | | | | |
| Oryza | 1 | B8A7J3 | A2WPL9 | 35/41 | 11/12 | 9/7 |
| (indica) | | | | | | |
| Zea mays | 3 | B4FD71 | B4FE31 | 35/37 | 11/11 | 10/10 |
| Sorghum | 3 | A0A1B6Q3F2 | C5XJV5 | 37/37 | 11/11 | 9/7 |
| bicolor | | | | | | |
| Eragrostis | | s4235_0.32-1 | s4235_0.42-1 | 37/38 | 11/11 | 11/6 |
| tef | | | | | | |

TABLE 9
Comparisons of predicted structures of TaPMP-
B3a and TaPMP-B4 homologs using Dali.

| | | | root mean | | |
|---|---|---|---|---|---|
| | | | square | Number of | |
| | | | deviation | structurally | Number of |
| | | | (rmsd) for | equivalent | residues |
| query | target | Z-score | αC atoms | residues | in target |
| TaPMP- | TaPMP-B3CS | 61.6 | 0.5 | 374 | 374 |
| B3a | Ta_3A_A0A077RP38 | 58.6 | 0.9 | 374 | 378 |
| | Ta_3D_A0A077RP03 | 44.7 | 1.8 | 346 | 375 |
| | Hv_3HG0183270.1 | 44.6 | 2 | 350 | 399 |
| | Os_B8A7J3 | 44.4 | 1.9 | 352 | 379 |
| | Zm_B4FD71 | 44.2 | 2.1 | 349 | 363 |
| | Sb_A0A1B6Q3F2 | 43.1 | 2.2 | 350 | 403 |
| | Et_s4235_0.32-1 | 42.9 | 2.1 | 346 | 392 |
| TaPMP- | TaPMP-B4CS | 44.3 | 5.1 | 405 | 406 |
| B4 | Ta_3A_A0A3B6E8W0 | 27.1 | 10.2 | 341 | 432 |
| | Ta_3D_3A0A0B4SVG8 | 25.6 | 13.8 | 350 | 429 |
| | Hv_3HG0183300.1 | 25.6 | 10.6 | 340 | 378 |
| | Os_A2WPL9 | 23.9 | 13.8 | 347 | 414 |
| | Zm_B4FE31 | 23.7 | 9.5 | 306 | 405 |
| | Sb_C5XJV5 | 20.1 | 4.6 | 270 | 354 |
| | Et_s4235_0.42-1 | 18 | 4.8 | 250 | 425 |

### REFERENCES

Abdullah et al. (1986) Biotechnology 4:1087.
Ayliffe et al. (2014) Methods Mol Biol 1127:159-172.
Barn et al. (2008) Proc S Afr Sug Technol Ass 81:508-512.
Barker et al. (1983) Plant Mol. Biol. 2: 235-350.
Begemann et al. (2017) Sci Rep. 7:11606.
Bernoux et al. (2008) Plant Cell 20:2252-2264.
Bevan et al. (1983) Nucl. Acid Res. 11: 369-385.
Capecchi (1980) Cell 22:479-488.
Che et al. (2018) Plant Biotechnol 16:1388-1395.
Chen et al. (2018) PLoS Genet 14, e1007287, doi:10.1371/journal.pgen.1007287.
Chen et al. (2019) New Phytol, doi:10.1111/nph.16169.
Clapp (1993) Clin. Perinatol. 20:155-168.
Coco et al. (2001) Nature Biotechnology 19:354-359.
Coco et al. (2002) Nature Biotechnology 20:1246-1250.
Comai et al. (2004) Plant J 37: 778-786.
Cesari et al. (2016) Proc Natl Acad Sci USA. 113: 10204-10209.
Curiel et al. (1992) Hum. Gen. Ther. 3:147-154.
De Coster et al. (2018) Bioinformatics 34:2666-2669.
De Oliveira et al. (2020) Font Genet. https://doi.org/10.3389/fgene.2020.00891.
Eggert et al. (2005) Chembiochem 6:1062-1067.
Eglitis et al. (1988) Biotechniques 6:608-614.
Ellis (2005) Theor Appl Genet 111:423-430.
Fujimura et al. (1985) Plant Tissue Cultural Letters 2:74.
Garfinkel et al. (1983) Cell 27: 143-153.
Graham et al. (1973) Virology 54:536-539.
Grant et al. (1995) Plant Cell Rep. 15:254-258.
Greenup et al. (2010) Plant Physiol 153:1062-1073.
Greve (1983) J. Mol. Appl. Genet. 1:499-511.
Haft et al. (2005) Computational Biology, PLoS Comput Biol 1(6):e60.
Harayama (1998) Trends Biotechnol. 16:76-82.
Henikoff et al. (2004) Plant Physiol 135: 630-636.
Hinchee et al. (1988) Biotech. 6:915
Holm (2020) Methods Mol. Biol. 2112:29-42.
Jinek et al. (2012) Science 337:816-821.
Jones et al. (2020) Protocols.io v3, dx.doi.org/10.17504/protocols.io.betdjei17506.
Joshi (1987) Nucl. Acid Res. 15: 6643-6653.
Lagudah et al. (1991) Genome 34: 387-395.
Langridge et al. (2001) Aust. J. Agric. Res. 52: 1043-1077.
Lemieux (2000) Current Genomics 1: 301-311.
Li et al. (2018) Bioinformatics 34, 3094-3100.
Liang et al. (2017) Nat Commun. 8:14261.
Liang et al. (2018) Plant Biotechnol J. 16:2053-2062.
Liang et al. (2019) Methods Mol Biol. 1917:327-335.
Liu and Godwin (2012) Plant Cell Rep. 31:999-1007.
Lu et al. (1993) J. Exp. Med. 178: 2089-2096.
Luo et al. (2016) Plant Cell Rep 35:1439-1450.
Ma et al. (2015) Molecular Plant 8: 1274-1284.
Mago et al. (2011a) Theor Appl Genet 123: 615-623.
Mago et al. (2011b) Theor Appl Genet 122:735-744.
Mago et al. (2014) BMC Plant Biol 14: 379.
Mago et al (2017) Wheat rust diseases: methods and protocols. Humana Press, pages 199-205.
Makarova et al. (2015) Nat. Rev. Microbiol. 13:722-736.
Mayjonade et al. (2016) BioTechniques 61, 203-205.
McFadden (1930) J Am Soc Agrc 22:1020-1034.
McIntosh et al. (1995) Wheat rusts: an atlas of resistance genes. CSIRO Publications, East Melbourne
Medberry et al. (1992) Plant Cell 4: 185-192.
Medberry et al. (1993) Plant J. 3: 619-626.
Moore et al. (2015) Nat Genet 47: 1494-1498.
Needleman and Wunsch (1970) J. Mol Biol. 45:443-453.
Ness et al. (2002) Nature Biotechnology 20:1251-1255.
Niedz et al. (1995) Plant Cell Reports 14: 403-406.
Ostermeier et al. (1999) Nature Biotechnology 17:1205-1209.
Ow et al. (1986) Science 234: 856-859.
Pattemore (2014) RNA extraction from cereal vegetative tissue. In: Robert J. Henry and Agnelo Furtado (eds.), Cereal Genomics: Methods and Protocols, Methods in Molecular Biology, vol. 1099, DOI 10.1007/978-1-62703-715-0_3, © Springer Science+Business Media New York 2014.
Petrie et al. (2020) Front P1 Sci, doi.org/10.3389/fpls.2020.00727
Pfaffl et al. (2002) Nucleic Acids Res 30: e36. doi: 10.1093/nar/30.9.e36.
Prasher et al. (1985) Biochem. Biophys. Res. Comm. 126: 1259-68.
Rajaram et al. (1988) Current CIMMYT approaches inbreeding wheat for rust resistance. In: Breeding strategies for resistance to the rusts of wheat. Eds Simmonds N W, Rajaram S. CIMMYT, Mexico; 101-118.
Richardson et al. (2014) Plant Cell Tis. Org. Culture 119:647-659.
Salomon et al. (1984) EMBO J. 3: 141-146.
Sieber et al. (2001) Nature Biotechnology 19:456-460.
Simkovi et al. (2008) BMC Genomics 9, 294. doi:10.1186/1471-2164-9-294.
Slade and Knauf (2005) Transgenic Res. 14: 109-115.
Singh and McIntosh (1984a) Can J Genet Cytol 26: 736-742.
Singh and McIntosh (1984b) Can J Genet Cytol 26: 723-735.
Singh et al. (2001) Slow rusting genes-based resistance to leaf and yellow rusts in wheat: Genetics and breeding at CIMMYT. Pages 103-108. In: Proc. 10th Assembly of the Wheat Breeding Soc. of Australia, Mildura, Australia. Wheat Breeding Society of Australia Inc., Australia.
Singh et al. (2011) Annu Rev Phytopathol 49: 465-481.
Stakman et al. (1962) Identification of physiologic races of Puccinia graminis var. tritici. Washington: USDA.
Stalker et al. (1988) Science 242:419-423.
Stemmer (1994a) Proc. Natl. Acad. Sci. USA 91:10747-10751.
Stemmer (1994b) Nature 370(6488):389-391.
Sun et al. (2016) Molecular Plant 9: 628-631.
Svitashev et al. (2016) Nat Commun. 7:13274.
Tabe et al (2019) Phytopathology 109: 1751-1759.
Thillet et al. (1988) J. Biol. Chem. 263:12500.
Toriyama et al. (1986) Theor. Appl. Genet. 205:34.
Tulpovi et al. (2019) Plant Genome 12:180065. doi:10.3835/plantgenome2018.09.0065.
Vaser et al. (2017) Genome Research 27:737-746.
Volkov et al. (1999) Nucleic Acids Research 27:e18.
Vrina et al. (2000) Genetics 156:2033-41.
Wagner et al. (1992) Proc. Natl. Acad. Sci. USA 89:6099-6103.
Wang et al. (1997) J Genet Breed 51:325-334
Wang et al. (1998) Acta Horticulturae 461:401-408.
Wick et al. (2017) PLoS Computational Biology 13:e1005595.
Woo et al. (2015) Nat Biotechnol. 33:1162-1164.
Zhao et al. (1998) Nat Biotechnol. 16:258-261.

1. A plant having a genetic modification(s) and an increased level of
i) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
ii) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's to 17,
when compared to a corresponding wild-type plant lacking the genetic modification(s), wherein the plant has enhanced resistance to one or more fungal pathogen(s) when compared to the corresponding wild-type plant.

2. The plant of claim 1, wherein the genetic modification(s) is an exogenous polynucleotide(s) encoding the polypeptide of part i) and/or ii).

3. The plant of claim 2, wherein the polynucleotide(s) is operably linked to a promoter capable of directing expression of the polynucleotide(s) in a cell of the plant.

4. The plant of claim 3, wherein the promoter directs gene expression in a leaf and/or stem cell.

5. The plant according to any one of claims 1 to 4, wherein the one or more fungal pathogen(s) is a rust or a mildew or both a rust and a mildew.

6. The plant of claim 5, wherein the rust is stem rust or leaf rust.

7. The plant of claim 5 or claim 6, wherein the one or more fungal pathogen(s) is a Puccinia sp., Blumeria sp., Fusarium sp., Magnoporthe sp., Bipolaris sp., Oidium sp., Gibberella sp., Cochliobolus sp., Exserohilum sp., Uredo sp. Microdochium sp., Helminthosporium sp., Monographella sp., Colletotrichum sp., Uromyces sp. or Erysiphe sp.

8. The plant according to any one of claims 1 to 7, wherein the polypeptide of part i) of claim 1 is encoded by a polynucleotide which comprises nucleotides having a sequence as provided in any one of SEQ ID NO's 18 to 26, a sequence which is at least 70% identical to one or more of SEQ ID NO's 18 to 26, or a sequence which hybridizes to one or more of SEQ ID NO's 18 to 26.

9. The plant according to any one of claims 1 to 8, wherein
a) the polypeptide of part i) of claim 1 comprises amino acids having a sequence which is at least 90% identical to one or more of SEQ ID NO's 1 to 9, and/or
b) the polynucleotide of part i) of claim 1 comprises a sequence which is at least 90% identical to one or more of SEQ ID NO's 18 to 26.

10. The plant according to any one of claims 1 to 9, wherein
a) the polypeptide of part i) of claim 1 comprises amino acids having a sequence which is at least 90% identical to SEQ ID NO:1 and/or SEQ ID NO:2, and/or
b) the polynucleotide of part i) of claim 1 comprises a sequence which is at least 90% identical to SEQ ID NO:18 or SEQ ID NO:19.

11. The plant according to any one of claims 1 to 10, wherein the polypeptide of part ii) of claim 1 is encoded by a polynucleotide which comprises nucleotides having a sequence as provided in any one of SEQ ID NO's 27 to 34, a sequence which is at least 70% identical to one or more of SEQ ID NO's 27 to 34, or a sequence which hybridizes to one or more of SEQ ID NO's 27 to 34.

12. The plant according to any one of claims 1 to 11, wherein
a) the polypeptide of part ii) of claim 1 comprises amino acids having a sequence which is at least 90% identical to any one of SEQ ID NO's 10 to 17, and/or
b) the polynucleotide of part ii) of claim 1 comprises a sequence which is at least 90% identical to any one of SEQ ID NO's 27 to 34.

13. The plant according to any one of claims 1 to 12, wherein
a) the polypeptide of part ii) of claim 1 comprises amino acids having a sequence which is at least 90% identical to SEQ ID NO:10, and/or
b) the polynucleotide of part ii) of claim 1 comprises a sequence which is at least 90% identical to SEQ ID NO:27.

14. The plant according to any one of claims 1 to 13 which comprises
a) at least two polynucleotides encoding the polypeptide comprising amino acids having a sequence as provided in in any one of SEQ ID NO's 1 to 9, a biologically active fragment thereof, or an amino acid sequence which is at least 70% identical to one or more of SEQ ID NO's 1 to 9, and/or
b) at least two polynucleotides encoding the polypeptide comprising amino acids having a sequence as provided in any one of SEQ ID NO's 10 to 17, a biologically active fragment thereof, or an amino acid sequence which is at least 70% identical to one or more of SEQ ID NO's 10 to 17.

15. The plant according to any one of claims 1 to 14 which comprises
a) at least two polynucleotides encoding the polypeptide comprising amino acids having a sequence as provided in SEQ ID NO:1 or SEQ ID NO:2, a biologically active fragment thereof, or an amino acid sequence which is at least 70% identical to SEQ ID NO:1 and/or SEQ ID NO:2, and/or
b) at least two polynucleotides encoding the polypeptide comprising amino acids having a sequence as provided in SEQ ID NO:10, a biologically active fragment thereof, or an amino acid sequence which is at least 70% identical to SEQ ID NO:10.

16. The plant according to any one of claims 1 to 15 which comprises i) and ii).

17. The plant of claim 16, wherein the polynucleotides are within 300 kb, 100 kb, 50 kb, or 20 to 30 kb of each other.

18. The plant according to any one of claims 1 to 17 which is a cereal plant.

19. The plant of claim 18, wherein the cereal plant is wheat, oats, rye, barley, rice, corn, sorghum or maize.

20. The plant according to any one of claims 1 to 19 which comprises one or more further genetic modifications encoding another plant pathogen resistance polypeptide.

21. The plant according to any one of claims 1 to 20 which is homozygous for one or more or all of the genetic modification(s).

22. The plant according to any one of claims 1 to 21 which is growing in a field.

23. A population of at least 100 plants according to any one of claims 1 to 22 growing in a field.

24. A process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence as provided in any one of SEQ ID NO's 1 to 9, a biologically active fragment thereof, or an amino acid sequence which is at least 70% identical to one or more of SEQ ID NO's 1 to 9,
ii) introducing the polynucleotide into a plant,
iii) determining whether the level of resistance to one or more fungal pathogen(s) is increased relative to a corresponding wild-type plant lacking the polynucleotide, and
iv) optionally, selecting a polynucleotide which when expressed produces a polypeptide which confers enhanced resistance to one or more fungal pathogen(s).

25. The process of claim 24, wherein
a) the polypeptide comprises amino acids having a sequence which is at least 90% identical to one or more of SEQ ID NO's 1 to 9, and/or
b) the polynucleotide comprises a sequence which is at least 90% identical to one or more of SEQ ID NO's 18 to 26.

26. The process of claim 24 or claim 25, wherein
a) the polypeptide comprises amino acids having a sequence which is at least 90% identical to SEQ ID NO:1 and/or SEQ ID NO:2, and/or
b) the polynucleotide comprises a sequence which is at least 90% identical to SEQ ID NO:18 or SEQ ID NO:19.

27. A process for identifying a polynucleotide encoding a polypeptide which confers enhanced resistance to one or more fungal pathogen(s) to a plant, the process comprising:
i) obtaining a polynucleotide operably linked to a promoter, the polynucleotide encoding a polypeptide comprising amino acids having a sequence as provided in any one of SEQ ID NO's 10 to 17 a biologically active fragment thereof, or an amino acid sequence which is at least 70% identical to one or more of SEQ ID NO's 10 to 17,
ii) introducing the polynucleotide into a plant,
iii) determining whether the level of resistance to enhanced resistance to one or more fungal pathogen(s) is increased relative to a corresponding wild-type plant lacking the polynucleotide, and
iv) optionally, selecting a polynucleotide which when expressed produces a polypeptide which confers enhanced resistance to one or more fungal pathogen(s).

28. The process of claim 27, wherein
a) the polypeptide comprises amino acids having a sequence which is at least 90% identical to any one of SEQ ID NO's 10 to 17, and/or
b) the polynucleotide comprises a sequence which is at least 90% identical to any one of SEQ ID NO's 27 to 34.

29. The process of claim 27 or claim 28, wherein
a) the polypeptide comprises amino acids having a sequence which is at least 90% identical to SEQ ID NO:10, and/or
b) the polynucleotide comprises a sequence which is at least 90% identical to SEQ ID NO:27.

30. The process according to any one of claims 24 to 29, wherein one or more or all of the following apply,
a) the plant is a cereal plant such as a wheat plant,
b) step ii) further comprises stably integrating the polynucleotide operably linked to a promoter into the genome of the plant, and
c) the plant of step iii) comprises a first polynucleotide encoding a polypeptide comprising amino acids having a sequence as provided in any one of SEQ ID NO's 1 to 9, a biologically active fragment thereof, or an amino acid sequence which is at least 70% identical to one or more of SEQ ID NO's 1 to 9, and a second polynucleotide encoding a polypeptide comprising amino acids having a sequence as provided in any one of SEQ ID NO's 10 to 17 a biologically active fragment thereof, or an amino acid sequence which is at least 70% identical to one or more of SEQ ID NO's 10 to 17.

31. A substantially purified and/or recombinant polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9.

32. The polypeptide of claim 31 which comprises amino acids having a sequence which is at least 70% identical, at least 80% identical, at least 90% identical, or at least 95% identical, to SEQ ID NO:1 and/or SEQ ID NO:2.

33. A substantially purified and/or recombinant polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's 10 to 17.

34. The polypeptide of claim 33 which comprises amino acids having a sequence which is at least 70% identical, at least 80% identical, at least 90% identical, or at least 95% identical, to SEQ ID NO:10.

35. An isolated and/or exogenous polynucleotide comprising nucleotides having a sequence as provided in any one of SEQ ID NO's 18 to 26, a sequence which is at least 70% identical to one or more of SEQ ID NO's 18 to 26, a sequence encoding a polypeptide of claim 31 or claim 32, or a sequence which hybridizes to one or more of SEQ ID NO's 18 to 26.

36. An isolated and/or exogenous polynucleotide comprising nucleotides having a sequence as provided in any one of SEQ ID NO's 27 to 34, a sequence which is at least 70% identical to one or more of SEQ ID NO's 27 to 34, a sequence encoding a polypeptide of claim 33 or claim 34, or a sequence which hybridizes to one or more of SEQ ID NO's 27 to 34.

37. A chimeric vector comprising the polynucleotide of claim 35 and/or the polynucleotide of claim 36.

38. The vector of claim 37, wherein the polynucleotide is operably linked to a promoter.

39. The vector of claim 37 or claim 38 which comprises one or more further exogenous polynucleotides encoding another plant pathogen resistance polypeptide.

40. A recombinant cell comprising an exogenous polynucleotide of claim 35 and/or claim 36, and/or a vector according to any one of claims 37 to 39.

41. The cell of claim 40, wherein the cell is a cereal plant cell such as a wheat cell.

42. A method of producing the polypeptide of claim 31 or claim 32, the method comprising expressing in a cell or cell free expression system the polynucleotide of claim 35.

43. A method of producing the polypeptide of claim 33 or claim 34, the method comprising expressing in a cell or cell free expression system the polynucleotide of claim 36.

44. A transgenic non-human organism, such as a transgenic plant, comprising an exogenous polynucleotide of claim 35 and/or claim 36, a vector according to any one of claims 37 to 39 and/or a recombinant cell of claim 40 or claim 41.

45. A method of producing the cell of claim 40 or claim 41, the method comprising the step of introducing the polynucleotide of claim 35 and/or claim 36, or a vector according to any one of claims 37 to 39, into a cell.

46. A method of producing a plant with a genetic modification(s) according to any one of claims 1 to 22, the method comprising the steps of
i) introducing a genetic modification(s) to a plant cell which increases the expression level of
a) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
b) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's to 17,
when compared to a corresponding wild-type plant cell lacking the genetic modification(s),
ii) regenerating a plant with the genetic modification(s) from the cell, and
iii) optionally harvesting seed from the plant, and/or
iv) optionally producing one or more progeny plants from the genetically modified plants,
thereby producing the plant.

47. The method of claim 46, wherein step i) comprises introducing a polynucleotide as defined in claim 35 or claim 36 and/or a vector according to any one of claims 37 to 39 into the plant cell.

48. A method of producing a plant with a genetic modification(s) according to any one of claims 1 to 22, the method comprising the steps of
i) crossing two parental plants, wherein at least one plant comprises a genetic modification(s) according to any one of claims 1 to 22,
ii) screening one or more progeny plants from the cross in i) for the presence or absence of the genetic modification(s), and
iii) selecting a progeny plant which comprise the genetic modification(s), thereby producing the plant.

49. The method of claim 48, wherein at least one of the parental plants is a tetraploid or hexaploid wheat plant.

50. The method of claim 48 or claim 49, wherein step ii) comprises analysing a sample comprising DNA from the plant for the genetic modification(s).

51. The method according to any one of claims 48 to 50, wherein step iii) comprises
i) selecting progeny plants which are homozygous for the genetic modification(s), and/or
ii) analysing the plant or one or more progeny plants thereof for enhanced resistance to one or more fungal pathogen(s).

52. The method according to any one of claims 48 to 51 which further comprises
iv) backcrossing the progeny of the cross of step i) with plants of the same genotype as a first parent plant which lacked the genetic modification(s) for a sufficient number of times to produce a plant with a majority of the genotype of the first parent but comprising the genetic modification(s), and
v) selecting a progeny plant which has enhanced resistance to one or more fungal pathogen(s).

53. A plant produced using the method according to any one of claims 46 to 52.

54. Use of the polynucleotide of claim 35 and/or claim 36, or a vector according to any one of claims 37 to 39, to produce a recombinant cell and/or a transgenic plant.

55. The use of claim 54, wherein the transgenic plant has enhanced resistance to one or more fungal pathogen(s) when compared to a corresponding wild-type plant lacking the exogenous polynucleotide and/or vector.

56. A method for identifying a plant which has enhanced resistance to one or more fungal pathogen(s), the method comprising the steps of
i) obtaining a sample from a plant, and
ii) a) screening the sample for the presence or absence of a genetic modification(s) which increases the level of
I) a polypeptide which confers enhanced resistance to one or more fungal pathogen(s), wherein the polypeptide comprises amino acids having a sequence as provided in any one of SEQ ID NO's 1 to 9, a biologically active fragment thereof, or an amino acid sequence which is at least 70% identical to one or more of SEQ ID NO's 1 to 9, and/or
II) a polypeptide which confers enhanced resistance to one or more fungal pathogen(s), wherein the polypeptide comprises amino acids having a sequence as provided in any one of SEQ ID NO's 10 to 17 a biologically active fragment thereof, or an amino acid sequence which is at least 70% identical to one or more of SEQ ID NO's to 17, and/or
b) screening the sample for the level of the polypeptide defined in I) and/or II).

57. The method of claim 56, wherein the screening comprises amplifying a region of the genome of the plant.

58. The method of claim 57, wherein the amplification is achieved using an oligonucleotide primer comprising a sequence of nucleotides provided as any one of SEQ ID NO's 43 to 55 and 62 to 68, or a variant thereof which can be used to amplify the same region of the genome.

59. The method according to any one of claims 56 to 58 which identifies a genetically modified plant according to any one of claims 1 to 22.

60. A plant part of the plant according to any one of claims 1 to 22, 44 or 53.

61. The plant part of claim 60 which is a seed that comprises the genetic modification(s).

62. A method of producing a plant part, the method comprising,
a) growing a plant according to any one of claims 1 to 22, 44 or 53, and
b) harvesting the plant part.

63. A method of producing flour, wholemeal, starch or other product obtained from seed, the method comprising;
a) obtaining seed of claim 61, and
b) extracting the flour, wholemeal, starch or other product.

64. A product produced from a plant according to any one of claims 1 to 22, 44 or 53 and/or a plant part of claim 60 or claim 61.

65. The product of claim 64, wherein the part is a seed.

66. The product of claim 64 or claim 65, wherein the product is a food product or beverage product.

67. The product of claim 66, wherein
i) the food product is selected from the group consisting of: flour, starch, leavened or unleavened breads, pasta, noodles, animal fodder, animal feed, breakfast cereals, snack foods, cakes, malt, beer, pastries and foods containing flour-based sauces, or
ii) the beverage product is beer or malt.

68. The product of claim 64 or claim 65, wherein the product is a non-food product.

69. A method of preparing a food product of claim 66 or claim 67, the method comprising mixing seed, or flour, wholemeal or starch from the seed, with another food ingredient.

70. A method of preparing malt, comprising the step of germinating seed of claim 61.

71. Use of a plant according to any one of claims 1 to 22, 44 or 53, or part thereof, as animal feed, or to produce feed for animal consumption or food for human consumption.

72. Use of a plant according to any one of claims 1 to 22, 44 or 53 for controlling or limiting one or more fungal pathogen(s) in crop production.

73. A composition comprising one or more of a polypeptide according to any one of claims 31 to 34, a polynucleotide of claim 35 or claim 36, a vector according to any one of claims 37 to 39, or a recombinant cell of claim 40 or claim 41, and one or more acceptable carriers.

74. A method of trading seed, comprising obtaining seed of claim 61, and trading the obtained seed for pecuniary gain.

75. The method of claim 74, wherein obtaining the seed comprises cultivating the plant according to any one of claims 1 to 22, 44 or 53, and/or harvesting the seed from the plants.

76. The method of claim 75, wherein obtaining the seed further comprises placing the seed in a container and/or storing the seed.

77. The method according to any one of claims 74 to 76, wherein obtaining the seed further comprises transporting the seed to a different location.

78. The method of claim according to any one of claims 74 to 77, wherein the trading is conducted using electronic means such as a computer.

79. A process of producing bins of seed comprising:
a) swathing, windrowing and/or or reaping above-ground parts of plants comprising seed of claim 61,
b) threshing and/or winnowing the parts of the plants to separate the seed from the remainder of the plant parts, and
c) sifting and/or sorting the seed separated in step b), and loading the sifted and/or sorted seed into bins, thereby producing bins of seed.

80. A method of identifying a compound that binds to a polypeptide comprising amino acids having a sequence as provided in any one of SEQ ID NO's 1 to 17, a biologically active fragment thereof, or an amino acid sequence which is at least 70% identical to any one or more of SEQ ID NO's 1 to 17, the method comprising:
i) contacting the polypeptide with a candidate compound, and
ii) determining whether the compound binds the polypeptide.


SEQUENCE LISTING (86 sequences)

SEQ ID NO: 1 - protein, 374 aa, Triticum aestivum cv. Hope
MDGLLNRSRGLAEGVAVMICPMLLALALNIVDLNDEVYGHGVPIALIAMAGFTLITGICSFMVCCFIQGFPRSSVILAIISSACLIMLAYFIAHFIIFKSVFITLGVLCGILLMLRTVCYLYDSSNGNVPEYTAIMQSVLDESHEFLTGVTGILFLGFQGLTLEGDVRTISRMGGEPMGYISFIFITLGVGSMLLAMTPPKSFGERKVVRLTLLLDYFMAAGIFLLLLVTMCKHTQLEMVALLVFVPPIICFSSLLFLVLFPVDGQDDEDPKPASLELTKVMFTGFLAVSIRTISNTSPNKLTGFFLLLSSMAIGFGLSWRLLSQNNTKSVLTTIDVSPVHVASAAKIASIFTHFCIVITTILLVAMASTASGK

SEQ ID NO: 2 - protein, 374 aa, Triticum aestivum cv. Hope
MVGLLNRSRGLAEGVAVMICPMLLALALNIVDLNDEVYGHGVPIALIAMAGFTLITGICSFMVCCFIQGFPRSSVILAIISSACLIMLAYFIAHFIIFKSVFITLGVLCGILLMLRTVCYLYDSSNGNVPEYTAIMQSVLDESHEFLTGVTGILFLGFQGLTLEGDVRTISRMGGEPMGYISFIFITLGVGSMLLAMTPPKSFGERKVVRLTLLLDYFMAAGIFLLLLVTMCKHTQLEMVALLVFVPPIICFSSLLFLVLFPVDGQDDEDPKPASLELTKVMFTGFLAVSIRTISNTSPNKLTGFFLLLSSMAIGFGLSWRLLSQNNTKSVLTTIDVSPVHVASAAKIASIFTHFCIVITTILLVAMASTASGK

SEQ ID NO: 3 - protein, 375 aa, Triticum aestivum
MICPVLLALALNLVDLNGEVYGHGVPIPLITMAGFTLITGICPLLACCFFERFLGLGVSPIPVTTSLATLSSCCLVVLACLIAQFIISKSIFVTVGVICGILVLVRTVCYYVHDTRNDDGREYTTDMHSVLDESHEFLTSVTGILFLGFEGLALNGHGQTGRKGEELVGYISFIVCTFGVCLMFLEMTPPPCFAVHRHGSQEERSEGEQQIVCLTLVLDCIMAVGIFVLLLVVMLKLTPLEFALWVLLPPAVSFGQLPVLVALKGNTQDEQESRPASLELTKATFTGFLAVSVSAISNASPSKLTRFFLLLSSMAIGFGLSWRLLSQINIRSGLAGCVSSAHVASAAKLASFCAHLCIVIATVLFVVMAGKASGK

SEQ ID NO: 4 - protein, 379 aa, Triticum aestivum
MLVGLLNRSRGLAEGVAVMICPVLLALALNIVDLNDEVYGHGVPIALIAMAGFTLITGICPFMVCCFLQGCPRSSVILAITSSACLVMLACFIAHFIIFKSIFITLGVICGIFLMLRTVCYYYYYLYDRPNGNVPEHTAIMQSVLDESHEFLTGITGILFLGFQGLTLDGDVRTISRMGGEPMGYISFIFITLGVGSMLLTMTPPKSFGERKVVRLTLLLDCFMAGGIFMLLLVTMRKHTQLEMVALLVFVPPIISFSSLLFLVLFPVDGQHDEDPKPASLELTKVTFTGFLAVSITTISNTSPNKFTAFFLLLSSMVIGFGLSWRLLSQNNTKSGLTNIDVSPVHVASAAKIASILTHFCIVITTILFVAMASTASGK

SEQ ID NO: 5 - protein, 379 aa, Hordeum vulgare
MICPVLLALALNIVDLNDKMYIHGLSIAMITMAGLTLITGITPLLVCCFSEGFPGSGVRHAPATTILATLSSCCLVVLACLIAQSIVSKPIFITVGVICGILVLLRTTCYYMYYTQNNDGRAYTIDMHNVLDESYEFLTGVTGILFLGFEGLALDGHVHTMGQKNGELVGYMTFIVCTFGVCLMFLEMTPPPCFAVDRHGSLEERRVGEEQIVCLTLILDCIMAVGIFVLLLVVMLKLTPFEVALWVLLPPVISFGQLPVLVALRGNTGVEQESRPASLELTKVTFTGFLAVSITTISTAGTASSRKLSGMFLLLSSMAIGFGLSWRLLSQANIRRGLASCVPSVHVVSAAKIASFCTHLCILIATFLFVVMAANASGK

SEQ ID NO: 6 - protein, 392 aa, Oryza sativa
MNRSRAFAEAVVIMVCPVLLAVALKKVDLKSQEHGRAVLIFMLVMAAFTLAFGTVPYLALSFSKRFSDHRWRLPAKATTWLAPSSCACLVGLACWIIHLILSARWAYAFPAMGAVFGLCIVVRTVSYCCRARGDPANLVAADERELPITRTALEDAMELERKLDESLELLAGVTALLFLALEGLALEGQINGGQRRLAAPMGVCFFACLFGVCFMLVETIPPPTPSRNDTGCRASIVRNLAEICDAFMALAIGAVMLSIMIVLVKLLALLLLSPLFLMLLVHAFDVAVGRDGGRDGDGDNVKPASLEVSKVTFTGFLTVAISARTTSRGPLSTSTEWFLIFAASAIVSGFAWRLLTHAKVGKSANVASFSTHLCIVLATVPFTVMAGQALLH

SEQ ID NO: 7 - protein, 399 aa, Zea mays
MAARDPMDAWMNRYRAFAEGVVVMVCPVLLAIALKKVDLKSEEHGGAVPTTMLVVAAVTLVAGICPYLVCCLSKRFFNNNGSSSGSPHAATMLLAPLSSTCLVALACWIVHLILDSWAFPAVGALVGLCSAIRTVMHFTTRAGQGDAEMEYCDRLESSLDFLVGITALLFLGLEGLALEGQINSSSGRLTTPIGTSFVVCVFAASLMLVETMPPRRLVRYLTETIDILTGFAVSLAMFFIMYPLMGLRALLLLAAPFLILMAYVFYVAIGKDDGNNNHQVASANGDEESSGVSSSRRSGAAADGDNKPASLELTKVTFAGFLAVSIPSISKGSVTIYSECFLHLAAAAVVSGLVWRLLTHYKKSQTTVATVADIASFCTHLCVAVAVIPFTIMAGNALS

SEQ ID NO: 8 - protein, 403 aa, Sorghum bicolor
MATSAIDPMDAWMNRYRAFAEGVVIMVCPVLLAIALKKVDLKSEEHGRAVPTTMLVVATVTLVAGICPFLVCCRFAKRFFNGSSLSSPHTVTMLLAPLSSTCLVALACWIIYLILHSWAFPVIGALIGLCSVIRTVTHFTTAARERNAATAMGCEEEYDCNKMESSLDFLAGITALLFLGLEGLALEGQINSTKAIIHDRLTKPIGTSFIVCVVCVGLMLLETMPPRKLIRNLTETIDIFTGFAVCSAMFFIMYALMKLRALLLLVAPFLILMMYVFYAATSKDKGNNNHRGASANGDEESPVISCSADGDKPASLELTKVTFTGFLAVSIPSISNGSVNMSTECFLHLAATAVVSGLIWRLLTHYKSQTTIATIADIASFCTHLCVAVAVIPFTLMAGKALS

SEQ ID NO: 9 - protein, 363 aa, Eragrostis tef
MAAGTSEMDAWMNRYRAFAEGVVVMVCPVLLAIALEKVDLKSKEHGLVIPVIMLVVAAVTLIAGIFPFLICRFPRERTGSPHAVTKFLAPLSSALLVVLACWIHFIILAKWSYTAIGAVLGASIVIPSGIYCYRRNDAAGGNVEQLENSLEFSAGITALLFLGLEGLALEGQITAMQDRLTKPLVASLFACIVGVCLMFVETTSLLIRNRNVIREMTRVFDFFMTCAIFFVMFFTIQELMKIQAVLLFAPPFLILMVLVYNNAISVQHGGNYELASLELTKVTFTGFLAVSIPSISKGSLNKGTHWFILLAAEAIVSGLGWRVLTHDKSAIFKKTAKVASFCTHLFIVIAAIPFTVMAGNALS

SEQ ID NO: 10 - protein, 406 aa, Triticum aestivum cv. Hope
MEWSQGSQQGKILSDAQTPPVPSGLPLVGKGEKSDVTSLCIIAFRFTLLSSVGNLLVVFHYDGTSKDTSELLTGTIEVAAYFYYLSTLAPSNVRPNARNFIDTGALVIMSYLLLLDINLSFFWLAILPTMAIVFITALYIKLNHHTGDSHGKVTPGSSNIDEKTIKTPMELRIIVVATFGALLVMDQLDDDTAGGFTISQFLLFLSSTVAALTCMVMKLPADRFLGSAPASELLQKTLLLLLLVTVHTLAAELLGEDVVLFCMPEVIPVLLWFSLNLDRPHHSPLTNIYKMKRHLKGLTIALGVLVVVPLFAYLVSSMDVSGLSYCTTIMVACGISGVLTYYIVFMLNYWHWPWQKQQAAAGSSSKDDVPSSCMLKFWADVLLIVAAVLLLLRYMVDVRLDLQQPL

SEQ ID NO: 11 - protein, 429 aa, Triticum aestivum
MGRSRASQRRQIMSKGGHARPPQPRSRLISKLEESDGALLCLIAATCTLSTSSVGNFQVFSLSDDGLTNKKYYELVTGTMEVAICYPYIFLKLHEFLPNLLPTPATPAPLKSWFACRPNVRIFGDTAVLVIISYLLLLGINLSFAWLAIFPVMAIVFIRALYMKLNRCRVTQGSSNVDEKSVKTPVELQIMVVATFGALLVMDQLDDDAAVGFSISQFLLFLSSMVAALTRMMMKLPADPFPGSAPASELLHKTLLILLLVTVHTVAVEWLGKDVVLFCMPEVVPVLLWCSLHLDRPRHSPLISVDKMKPYMKGIIALCALVAAPLFAYLVNSMDVSGLSGCTRIMVSCGVLGVLTYYLVFMLRHWPCPGQKQGGSSKDDVPSSGMLKFWAEALLMAATFLLLLRYLVSVRLGLQQSLVGTLCRNVQRL

SEQ ID NO: 12 - protein, 378 aa, Triticum aestivum
MEWSQGSQQGKILSHAQTPPVPSGLPLVGKVEKSDVTSLCIIAFRFTLLSSVGNLLVVFHHDGTSKDTSELLTGTMEVVVCFYYLSTLAPSNVRPNARNFIDTGALVIMSYLLLLDINLSFLWLAILPTMAIVFITALYIKLNHHTGDSHGKATPGSSNIDEKTIKTPMELRIIVVATFGALLVMDQLDDDTAGGFTISQFLLFLSSTVAALTCMVMKLPADRFLGSAPASELLQKALLLLLLVTVHTLAAELLGEDVVLFCMPEIIPVLLWFSLNLDRPHHSPLTNIYKMKPHMKGLTIAKGVLVVVPLFACLVSSMDVSGLSYCTTIMVACGISGVLTYYIVFMLNYWHWPWQKQQAAAGSSSKDDVPSSGVLACS

SEQ ID NO: 13 - protein, 432 aa, Hordeum vulgare
MGRSRGSQHREMVSHNENAHRSPPRSRLISKLEESDGQSLCLVAAACTLLSTVCNFQVFLLSDDDGFTNKEISEFVTGTMEFAICYSYIFLKLHELILPNFAPLKSWIKWGRNFRIFRDTGVFVTISYLLLLDISWRFVSLAIFPVMAIAFIGALYFKLDPHTGDISRKVTGRSSKVDEKTIRTPVKLQIIVVATFGALLVMDQLDDDTAMGFAISQFLLFLSSTVAALTRMMIKLPTGPFPGSAPAAELLHKTLLLLLLVTAHTLAAEWLGEDVVLFCMPEVVPVLLWYSLHLDRPHHSPLISVVKMKPHMKGLIALCTLVVAPLFAYMVNSMDVSGLSWCTRIMVSCGVSGALTYYLVFMLRSWHWPRQREPAAAGSSKDDVVSSGMLKFLANALLITAALLLLLWYMVSVRLGLETALVDTLRRNLQQL

SEQ ID NO: 14 - protein, 405 aa, Oryza sativa
MELELVYLYPVTLVPAVVGRFSILASPLLREFEEHNLQFTSIIPLFFFYSYISKFLSDLQGQPLPLVKLYARALHRFTSCSSSKSRASLRIYMDTAVVVVLSYLSLLKINLSYIWLAVFPTLTLAFIAALFNEELRGDRRAAVAEEDDGRRRKDHSLELKAMAVVPYWVLCAMGQFQGGDSFAVSQFLLFLGSTLGALALMAARLSQLAGAAPGLAPASELLRKATLVVLLVTAHAVAAELLGEATVALLCLPELAPALFWFTLHLDGGSSPAAAIDDIKSHRNALSVLAVAVVAYLATAMGERGLSVSTITMVSCGVSGLLVRCAVLVLGQWPGQAETAGSTAASLMEVVRVLKFWGNTLLAVAAMLLLLALLTAFRLCLQEPILPTVAKCFSDYIDKVPQMLV

SEQ ID NO: 15 - protein, 354 aa, Zea mays
MDSKKMYVCSLPLMPYVVFGFVGSQLPSAPNWQSYKVDDPNAPRMILLVILYIYVILLAKIKNDRIYIETATAVVISYVSLLQISWSYIWLVPFPIVAIGFIVALCKELSHEDEAGGSHSQEQSNEGEGEATTSSWEKVEGLAAVTVVPYWALLIMGLFHPDKFAVSQFLLFFSFMLGALTMMMTRLVETGRAIAHRGIVPASELLWKASLVVLLVAVHAVAAELLGENALLLCMPEILPVLLWLSVRLDTADDRIKLVLDSNLLPAALPAVLAMLAKGEPLLYWCRKALVSCVVSALLTFYVYFMLCQWPGGQRDRTLTGSSLQHALIHLKSWAKVLLKAVAVLLIFVCILSN

SEQ ID NO: 16 - protein, 414 aa, Sorghum bicolor
MDSKKVYVWSLPLTPEVILSFVSSQVALLMQSYKEHELYVTTVIIPLVILYMNACSFVNLGLPLPLSPLNFLPAKKVWIYIETAIVVVISYVSLLIVSWSYIWLVVFPIIAIGFIVALCSELSHQDQAGGSHSQEQSNEGEGDEATAPVGKKAEGLEAVALVPYWALCMMGHFHADKFTVSQFLLFFSFMLGALTMMMTRLVGLATVHRGIAPASELLRKASLVVLLVAVHAMAAELLGENALLFCLPEIVPVLLWLSVRLDCHDGAIITGADRIKLGMDSRVVNLLGAAAAAVLAAMLAAGYMDESVLYWCGKALVSCAVSALLTLYIVFMLCQWPGGQRDGTLVTVTAASLLQEAIQLLNSWAKVLLAAVATLLVLTTLAAFQLVLLQELAASATADQILQRLGQKFLQSII

SEQ ID NO: 17 - protein, 424 aa, Eragrostis tef
MDSKMVNMSSQPFAKEPVISLLIISLVTPLIQAKEDNQQYVTKIAPMLLGCFISLYVQFWSYFKRLTEYSTDFTSSNNNCIYMEAAGFVATSYVSLLLANWTNIWLAIFPIIAVGFILALCYELRHQRSNQPDDGEGNASSSGGVQGGQNEIKLAEVHKAAALVPYWLLCVMWQFHTAENFAVSQFLLFFSSMLGALTLMLKQLALTAGAPPGIAPASVLLRKASLVVLLVTVHAFSAELLGENVMLFILPEILPVILWFSISLNRRDSSYITADKIRAQRSVITVLGAAVAAVFAFLAFSMDEFVLSWCTKAMVSCGVSGLLVYHVVFILCQWPELDSTASSFVEAAFDLLKLWAYVLLNAAAGLLAFALVGVVRLGLHKQLLAQPKCYRQEPHILERDPAASAAGAGTIGKIIGTHKIILVV

SEQ ID NO: 18 - DNA, 1122 bp, Triticum aestivum cv. Hope
atggatggactattgaaccgttcccgtggattggcggaaggagtggcggttatgatatgcccaatgcttctcgcacttgctctaaacatagttgacctcaatgatgaagtgtacggacatggagtccccattgccttgattgccatggcaggctttactttgatcaccggcatttgctccttcatggtgtgctgcttcatacaagggttcccgagaagtagcgtgatcctggcaatcatttcgtctgcttgcctcatcatgctcgcctacttcattgcacacttcattatcttcaaatccgtttttatcacccttggagtcctgtgtggaattttgctcatgctgcgaaccgtctgctacttgtatgatagctcaaatggcaatgttccagaatacacggccataatgcaaagcgtactggacgaatcacatgagttcttgactggcgtcactggaattctttttctggggttccaaggtttgactctggaaggtgacgttcggacaataagtcgcatgggcggggaaccaatgggttacatcagcttcatcttcatcactctaggtgtgggatcaatgctcctcgcgatgaccccacctaaaagttttggagagagaaaagttgtgcgtttgactctcctccttgactacttcatggcggctggtatcttcctgctattgttggttaccatgtgtaagcacacgcagctggagatggttgctctgttggtctttgtgcctccgattatatgcttcagttcacttctgttcctggtcttgttcccggtggacggacaagatgatgaagaccccaagccagcgtcactggaactgacaaaggtcatgttcaccggattcttggccgtttcaataagaaccatcagcaacacttcaccgaacaagctcaccggattcttcttactattatcctccatggctattgggtttggactctcatggaggctcctttcacagaacaacacaaagagtgttctcactactattgatgtttcacctgttcatgttgcttctgccgccaagattgcttccatttttacccacttttgtattgtaatcaccacaattctacttgtcgcaatggctagtactgccagcggtaaa

SEQ ID NO: 19 - DNA, 1122 bp, Triticum aestivum cv Hope
atggttggactattgaaccgttcccgtggattggcggaaggagtggcggttatgatatgcccaatgcttctcgcacttgctctaaacatagttgacctcaatgatgaagtgtacggacatggagtccccattgccttgattgccatggcaggctttactttgatcaccggcatttgctccttcatggtgtgctgcttcatacaagggttcccgagaagtagcgtgatcctggcaatcatttcgtctgcttgcctcatcatgctcgcctacttcattgcacacttcattatcttcaaatccgtttttatcacccttggagtcctgtgtggaattttgctcatgctgcgaaccgtctgctacttgtatgatagctcaaatggcaatgttccagaatacacggccataatgcaaagcgtactggacgaatcacatgagttcttgactggcgtcactggaattctttttctggggttccaaggtttgactctggaaggtgacgttcggacaataagtcgcatgggcggggaaccaatgggttacatcagcttcatcttcatcactctaggtgtgggatcaatgctcctcgcgatgaccccacctaaaagttttggagagagaaaagttgtgcgtttgactctcctccttgactacttcatggcggctggtatcttcctgctattgttggttaccatgtgtaagcacacgcagctggagatggttgctctgttggtctttgtgcctccgattatatgcttcagttcacttctgttcctggtcttgttcccggtggacggacaagatgatgaagaccccaagccagcgtcactggaactgacaaaggtcatgttcaccggattcttggccgtttcaataagaaccatcagcaacacttcaccgaacaagctcaccggattcttcttactattatcctccatggctattgggtttggactctcatggaggctcctttcacagaacaacacaaagagtgttctcactactattgatgtttcacctgttcatgttgcttctgccgccaagattgcttccatttttacccacttttgtattgtaatcaccacaattctacttgtcgcaatggctagtactgccagcggtaaa

SEQ ID NO: 20 - DNA, 1125 bp, Triticum aestivum
atgatatgcccggtgcttctcgcactggctctcaacttagttgacctcaatggcgaggtgtacggacatggtgtccctataccgttgatcaccatggcaggctttactttgataactggcatctgtcccttgctcgcatgctgcttctttgagcgattcttcggcctgggcgtaagtcctatccccgtgaccacgagtttggcaaccctttcgtcttgttgcctcgtcgtcctcgcctgcttgattgcacagttcattgtctccaaatccatttttgtcactgttggagtcatctgtggaatcttggtcttggttcggaccgtctgctactacgtgcacgacacacggaatgacgatggtcgggaatacacaacggacatgcacagtgtactggatgaatcacatgagttcttgaccagtgtcactggaattctttttctggggttcgaaggtctggccctcaatggtcacggtcagacgggtcgcaagggcgaggaattagtgggctacatcagtttcatcgtctgcacttttggagtgtgcttaatgttcctcgagatgaccccacctccatgttttgcggtacataggcatggtagccaagaggaaagaagtgagggagagcaacaaattgtgtgtttgactctcgtcctagattgcatcatggcggttggtatctttgtactattgttggtcgtcatgcttaagctcacaccgcttgagtttgccttgtgggtcttgctgcctccggctgtaagcttcggtcaacttccggtcctggttgcattgaagggaaacacacaagatgaacaagaatcaaggccggcgtcgctggaactgacaaaggccacattcactggattcttggccgtgtcggtaacagccataagcaatgcttcacccagcaagttgaccagattttttctactattatcttccatggctattgggtttggtctctcatggaggctcctatctcagattaacataaagagtggtctcgccagttgtgtttcatctgcacatgttgcttccgctgccaagcttgcttccttctgcgctcatttatgcattgtaatcgctacagttctattcgtagtaatggctggtaaagcaagtgggaaa

SEQ ID NO: 21 - DNA, 1134 bp, Triticum aestivum
atggttggactattgaaccgttcccgtggcttggcggaaggtgtggcggttatgatatgcccagtgcttctcgcacttgctctaaacatagttgacctcaatgatgaagtctacggacatggagtccccattgccttgattgccatggcaggctttactttgatcaccggcatttgccccttcatggtgtgctgcttcttacaagggtgccccagaagtagcgtgattctggcaatcacttcgtctgcttgtctcgtcatgctcgcctgtttcattgcacacttcattatcttcaaatccatttttatcacccttggagtcatctgtggaattttcctcatgctgcgaaccgtctgctactactactactacttgtatgatagaccaaatggcaatgttccagaacacacggccataatgcaaagcgtactggatgaatcacatgagttcttgaccggcatcactggaattctttttctggggttccaaggtttgactcttgatggtgacgttcggacaataagtcgcatgggcggggaaccaatgggttacatcagcttcatcttcatcactctaggtgtgggatcaatgctcctcacgatgaccccacctaaaagttttggagagagaaaagttgtgcgtctgactctcctccttgattgcttcatggcgggtggtatcttcatgctattgttggttaccatgcgtaagcacacccagcttgagatggttgccctgttggtctttgtgcctccgattataagcttcagttcacttctgttcctagtcttgttcccggtggatggacaacatgatgaagaccccaagccagcgtcactggaactgacaaaggttacattcaccggattcttggccgtgtcgataacaaccatcagcaacacttcgccgaacaaattcaccgcattcttcttactattatcctccatggttattgggtttggactctcatggaggctcctttcacagaacaacacaaagagtggtctcactaatattgatgtttcacctgttcatgttgcttctgccgccaagattgcttccattttgacccacttttgtattgtaatcaccactattctatttgtcgcaatggctagtactgccagcggtaaa

SEQ ID NO: 22 - DNA, 1137 bp, Hordeum vulgare
atgatatgcccagtgcttcttgcgttggctctcaacattgtcgatctcaacgacaagatgtacattcatggtctctccatagccatgatcaccatggcaggccttactttgataactggcattacccccttgctcgtttgttgcttctcggagggtttccctggctcaggcgtaagacatgcgcccgcaacaacaatcctggcaaccctttcgtcatgttgccttgtcgtcctcgcatgcttgattgcacaatctattgtctccaaacccatttttatcacggttggagtcatctgtggaatcttggtccttcttcgaactacctgctactacatgtattatacacaaaacaacgatgggcgggcatacacgattgatatgcacaatgtactagatgaatcatatgagttcttgaccggggtcactggaattctctttctagggttcgaaggtctagcactggatggtcacgtccacacgatgggtcaaaagaacggggaattagtgggctatatgactttcatcgtctgcacatttggtgtgtgcttaatgtttttggagatgaccccacctccatgttttgcggtagataggcatggtagccttgaagaaaggagagtgggagaggaacaaattgtgtgtttgactctcatcctagactgcattatggctgttggtatattcgtactactgctcgttgtcatgcttaagctcacgccgtttgaggttgccctatgggtattactaccaccggttataagcttcggtcaacttccagttctagtcgcgttaagagggaacacaggagttgaacaggaatctcgacctgcatcactagaacttacaaaggtcaccttcactggattcttggcggtgtcgataacaactatcagtaccgcaggcaccgcttcgtccagaaagttgagcggaatgttcctactattatcttcgatggctattgggtttggtctttcatggaggctgctgtcccaggctaacataaggagaggtctggccagttgtgttccatctgttcatgttgtttctgctgccaagattgcatcattttgcactcacttatgcattttaatagctacatttctatttgtagtaatggctgctaatgcaagcggcaaa

SEQ ID NO: 23 - DNA, 1176 bp, Oryza sativa
atgaaccggtcccgcgccttcgcggaggcggtggtgatcatggtgtgcccggtgctcctcgccgtggccctcaagaaggtcgacctgaagagccaggagcacggccgcgccgtgctgatcttcatgcttgtcatggcagccttcaccctcgcattcggcaccgtcccctacctcgctctctccttctctaagaggttcagcgaccacaggtggagactccccgccaaggcgaccacctggctggctccctcgtcgtgcgcctgcctcgtcggcctcgcgtgctggatcatacacctcatcctctccgcgcgctgggcttacgcgttccccgccatgggcgccgtgttcggcctctgcatcgtggtccgcaccgtcagctactgctgccgtgcacggggtgacccggctaatctggtagctgccgacgagcgagagctacctatcaccagaactgctttagaggatgccatggaactggaacgcaagctggacgagtcgctggagttgttggccggcgtcacggcgctgctgttcctggcgctggaagggctggcgctggagggccagatcaacggcggccagcgtcgcctcgccgcgcccatgggcgtgtgcttcttcgcctgcctgttcggcgtctgcttcatgctcgtcgagaccatcccgccgcccacgccgagcaggaacgacacgggatgccgcgccagcatcgtgaggaatctcgccgagatctgcgacgccttcatggcccttgccatcggtgcggtcatgctgtcgatcatgatcgtgctcgtgaagctactggcgctgctgctgctatccccgctcttcctgatgctgctggtgcacgcgttcgacgtcgccgtcggccgcgacggcggcagagacggcgacggcgacaatgtgaagccggcgtcgctggaggtgagcaaggtcacgttcaccgggttcctgaccgtggcgatatctgccaggacgaccagccgtggcccgctcagcacgtccaccgagtggttcctcatctttgcagcgtccgccattgtttcgggcttcgcttggaggctcctgacgcacgccaaagttgggaaaagtgccaatgtagcttccttctccacgcatttgtgcatcgtgcttgcaacggtgccatttacagttatggccggacaggctctgttgcac

SEQ ID NO: 24 - DNA, 1197 bp, Zea mays
atggctgccagagatcccatggatgcatggatgaaccgttaccgtgctttcgcggaaggagtggtggtgatggtctgcccggtgcttcttgcgatagcgctgaagaaggttgacctgaagagcgaagagcatggtggtgccgtcccgaccaccatgctcgtggtagcagcggttactctagtcgccggtatttgcccctacctcgtctgctgtctctccaagagattcttcaataataatgggagctcctcaggttccccacacgctgctaccatgttgctggcacccttatccagcacatgtttagtggcacttgcctgttggatcgtacatctcatcctagacagctgggcgttccctgcggtcggcgccctcgtcggactctgctccgcgattcgtaccgtcatgcacttcacaacccgtgcgggacaaggagatgctgaaatggagtactgcgatagactggagagctcgcttgatttcttggttggtataactgcgcttctgtttctgggactggaaggcctcgctctggagggtcagattaacagctccagcggtcgtcttaccacgccgatcggcacaagcttcgttgtctgtgtttttgctgcgagcttaatgctcgtggagacgatgcctccacgcagactcgtgaggtatctgacagagaccattgatatcctcacgggttttgccgtctccctggctatgttcttcatcatgtatccactcatgggactacgagcattgcttctgctagcagctccgttcctgatcctcatggcgtatgtcttctacgtcgctatcggcaaggatgatggcaacaacaaccaccaggttgctagcgcgaacggcgatgaagaatcgtcaggtgttagcagcagccgcaggagtggtgcggcggcggacggtgataataaaccagcgtcgcttgaactgacaaaagtaacattcgcagggttcctggcggtgtcgataccaagcatcagcaaaggttcagtcaccatttattccgaatgtttcttgcaccttgcagcagcagctgttgtctcaggcctcgtgtggaggctcctaacgcattacaaaaagtcacagaccaccgtcgcaactgttgcagatattgcttccttctgcacccatctctgtgttgcagttgcggtaattccgtttacgataatggccgggaatgcactctca

SEQ ID NO: 25 - DNA, 1209 bp, Sorghum bicolor
atggctaccagtgccatagatccgatggatgcatggatgaaccgttaccgtgctttcgcggaaggtgtggtgattatggtctgcccggtgcttcttgcgattgcactgaagaaggttgacctgaagagcgaggaacatgggcgtgccgtcccaaccaccatgcttgtggtagcgacggtcaccttagttgccggtatttgccccttccttgtctgctgtcgtttcgccaagagattcttcaatgggagctcgttaagttccccacacactgttaccatgttgctggcgcctttatccagcacatgtttagtggcacttgcctgttggatcatatacctcatcctacacagctgggcattccctgtgatcggtgccctcattggactctgctccgtgatccgtaccgtcacgcacttcacaaccgctgcgagagaaagaaatgctgcaactgcaatgggatgtgaggaggagtacgactgcaataaaatggagagttcacttgatttcttggctggtataactgcactgctgtttctgggactggaaggcctggctctggagggtcagattaacagcaccaaagcaataatacacgatcgtcttaccaaaccaatcggcacaagcttcattgtttgtgttgtttgtgtgggattaatgctccttgagacgatgcctcctcgcaaactcataagaaacctgacagagactatagatatttttacgggttttgccgtctgttcggctatgttcttcattatgtacgcactcatgaaactgagagcgttgcttctgctagtagctccgttcctgatcctcatgatgtatgtgttctatgcggctaccagcaaggataagggcaacaacaatcacaggggtgctagtgctaacggtgatgaagaatcacctgttattagctgtagtgcggacggtgataaaccagcgtcacttgagctaacaaaggttacatttactggattcctggcggtgtcgataccaagcatcagcaatggttcagtcaacatgtctaccgaatgtttcttgcaccttgcagcaacagctgttgtctcaggtctcatatggaggctcctaacgcattacaagtcacagaccaccattgcaactattgcagatattgcttctttctgcacccatctctgtgttgcagttgctgtaattccatttacattaatggccgggaaggctctctca

SEQ ID NO: 26 - DNA, 1089 bp, Eragrostis tef
atggctgccgggacttctgagatggatgcttggatgaaccgttaccgagctttcgccgaaggagtggtggtcatggtctgcccagtgctgcttgcaattgcactcgaaaaggttgatctgaagagcaaggaacatgggcttgtgatcccagtcatcatgcttgtggtagctgctgttaccttaatagccggtatttttcccttcctcatctgccgttttcccagagaaaggacaggatcccctcacgctgtgacaaagtttttggctcccttatccagtgcactcttagtggtacttgcctgctggatacactttattatcttggccaaatggtcatacactgcaattggtgctgtacttggagcaagcatcgtgattcctagtggcatatactgctaccgtagaaacgatgctgcaggagggaacgtagaacagctagagaactcacttgagttctcggctggtataactgcactgctgtttctaggactggaaggcttggcgttggagggtcaaatcacggcaatgcaagatcgtcttaccaaaccgttggtcgcaagcttgttcgcttgtattgttggcgtgtgcttaatgttcgttgagacaacttcccttctcataagaaatcgcaacgtgatacgtgagatgactagggtctttgatttcttcatgacatgtgccatcttcttcgtcatgttcttcaccatccaagaactcatgaagatacaagcagtgcttctatttgcaccaccattcttgatcctcatggtgcttgtatataataatgccatcagtgtgcaacacggtggaaattatgaactggcgtcacttgaactgaccaaagtaacatttaccgggttcctggccgtgtcgataccaagcataagcaaaggttcgctcaacaagggtacacactggttcatactccttgctgcagaggctattgtttcaggtctgggatggagagtgctgacacatgacaagtcagccatctttaagaaaactgcgaaagttgcttccttttgcacccacctcttcattgttattgctgcaattccattcacggtcatggccggaaatgctctctct

SEQ ID NO: 27 - DNA, 1218 bp, Triticum aestivum cv. Hope
atggagtggagccaaggatctcagcagggaaaaatattgtcagacgctcagacacctccagttccttcaggattgccattggttggcaagggtgaaaaatcggatgtaacgtcgttatgcataatagcattcaggttcacactgttgagcagtgttggcaatttactagtggtatttcattatgatggtaccagcaaagacacaagtgagctgctaactggcacaattgaggttgctgcttacttctattatttgagcacattagcgccttcgaacgtgcgccccaacgcacggaatttcatagacacaggcgctcttgttattatgtcttatctattattactggacatcaacttgagctttttctggctcgccatcttgcccaccatggccattgttttcataacagcgctttacatcaagctcaatcatcatactggtgatagccatggaaaagttactccaggatcttctaacatcgacgagaagaccatcaagacaccgatggagcttcggatcatagtggtggcaactttcggggctctcctagtcatggatcagctcgacgacgatacagctggtgggttcaccatctcccagttcctcctattcctgagctccacggtggcggcactgacgtgtatggtgatgaagctgcctgccgaccgttttttgggcagtgctccggcgtcagagttgctccaaaagaccttgcttctgctcctactggtgacggtgcacacattggccgcggagttgttgggcgaggacgtggttctgttctgcatgccagaggtcatcccggtgctcctctggtttagccttaacctcgaccgcccgcaccacagcccgctcaccaacatctacaaaatgaagcgacacctgaagggactcactattgccctaggtgtactggtggtagttcctcttttcgcgtacttagtgagctccatggatgtatctgggctctcgtactgcacgacgatcatggtggcatgtggcatctcaggggttctgacctactacattgtattcatgctaaactactggcattggccgtggcagaagcaacaagcagcagctggcagcagcagcaaggacgatgtgccttcctcatgtatgctcaagttttgggcagatgttttactgatagtggcggcagtgctcctgctgctaaggtatatggttgatgtccggcttgatctccaacaaccacta

SEQ ID NO: 28 - DNA, 1287 bp, Triticum aestivum
atggggcggagccgagcatcccagcgccgacaaatcatgtcaaaaggcggacatgctcggccgccacagcctcgttcgcgattaatcagcaagcttgaggaatcagatggagcattattatgcctaatagcagcaacctgcacactgtccaccagcagcgtcggcaatttccaggttttctcattaagtgatgatggtcttaccaacaaaaaatactatgagttggtaactggcacaatggaggttgctatttgctacccttatatatttctgaaattgcacgagttcttgcctaatctgcttccgacgccggctacaccggcgccattgaagtcgtggttcgcatgccgtcctaacgtccggattttcggagacacggccgttcttgtgattatatcttatctgctattactgggcatcaacttgagcttcgcctggctcgccatctttcccgtcatggccattgttttcatacgagcgttgtacatgaaacttaatcgctgtagagtaacacaaggatcttctaacgttgatgagaaatccgtcaagacacctgttgagcttcagattatggtggtggcgacgttcggggcgctcttggtgatggatcaactcgacgacgatgcagctgtggggttctccatctcccaattcctcctgttcctgagctccatggtggcggcacttacgcgaatgatgatgaagctgcccgctgacccttttccgggcagcgcaccggcgtcagagttgctccacaagaccttgcttatcctcctgctggtgacggtgcacacggtcgccgtggaatggctgggcaaggacgtggttctgttttgcatgccagaggtagtcccggtgctcctctggtgcagccttcaccttgaccgcccacgccacagcccgctcatcagcgtcgacaagatgaagccatacatgaaagggatcattgccctgtgtgcactggtggcagctcctcttttcgcatacctggtgaactccatggatgtttctgggctctccgggtgcacgaggataatggtgtcatgtggcgtcttaggggttctgacctactaccttgtgttcatgctacgccactggccgtgtccggggcagaagcaaggtggcagcagcaaggacgatgtgccttcctccggcatgctcaagttttgggcagaggctttactcatggcggccacatttctgctgctgctaaggtatctggtttctgtccggcttggtctgcaacaatcactggttggtacactctgtcgaaatgtgcaacggcta

SEQ ID NO: 29 - DNA, 1173 bp, Triticum aestivum
atggagtggagccaaggatctcagcagggaaaaatattgtcacacgctcagacacctccagttccttcaggattgccattggttggcaaggttgagaaatcggatgtaacgtcattatgcataatagcattcaggttcacactgttgagcagtgttggcaatttgctagtggtatttcatcatgatggtaccagcaaagacacaagtgagctgctaactggcacaatggaggttgttgtttgcttctattatctgagcacattagcgccttcgaacgtgcgccccaacgcacggaatttcatagacacaggcgctcttgttattatgtcttatctgttattactggacatcaacttgagctttctctggctcgccatcttgcccacaatggccattgttttcataacagcgctttacatcaagctcaatcatcatactggtgatagccatggaaaagctactccaggatcttctaacatcgacgagaagaccatcaagacaccgatggagcttcggatcatagtggtggcgacttttggggctctcctagtcatggatcagctcgacgacgatacagctggtgggttcaccatctcccagttcctcctattcctgagctccacggtggcggcattgacatgcatggtgatgaagctgcctgccgatcgttttttgggcagtgctccggcatcagagttgctccaaaaggccttgcttctgctcctactggtgacggtgcacacattggccgcggagttgttgggcgaggacgtggttctgttctgcatgccagagatcatcccggtgctcctctggttcagccttaatctcgaccgcccacaccacagcccgctcaccaacatctacaaaatgaagccacacatgaagggactcactattgccaaaggtgtcctggtggtagttcctcttttcgcgtgcttagtgagctccatggatgtatctgggctctcctactgcacgacgatcatggtggcatgtggcatctcaggggttctgacctactacattgtattcatgctaaactactggcattggccgtggcagaagcaacaagcagcagctggcagcagcagcaaggacgatgtgccttcctcaggtatgctagttttcctttgttttgtggtctttgtaggtgtattggcatgttcatga

SEQ ID NO: 30 - DNA, 1296 bp, Hordeum vulgare
atggggcgaagccgaggatctcagcaccgagaaatggtgtcacacaacgaaaacgctcatcggtcaccacctcgttcacgattgatcagcaaactggaagaatctgatggacaatcgttatgcctagttgcagcagcctgcacactgctcagcactgtctgcaatttccaagtatttctattaagtgatgatgatggttttaccaacaaagaaattagtgagtttgtaaccggcacaatggaatttgctatttgctactcctatatatttttgaaattgcacgagttgatcttgcccaactttgcacccttgaagtcgtggatcaaatggggtcgtaacttccggattttcagagacactggcgtttttgtgactatatcctacctgttattactggacatcagttggagattcgtctcgcttgccatctttcccgtcatggccattgctttcataggagcactgtacttcaagctcgatcctcatacaggcgacatcagtcgcaaagttacggggcgatcttctaaggtcgacgagaaaacaatcaggacacctgtgaagcttcagatcatagtggtggcaacattcggggcgctgttggtgatggatcaacttgatgatgatactgctatgggcttcgccatctcgcagttcctcttgttcctgagctccacggtggcggcgctgacgcgcatgatgataaagctgcccactggccctttcccgggtagtgcaccggcggcagagctgctccacaagacattgcttctacttctactggtgacggcacacacattagccgcggagtggctgggcgaggatgtggttctgttctgcatgccggaggttgtcccggtgctcctctggtacagccttcacctcgaccgcccacaccacagcccgctcatcagcgtcgtcaaaatgaagccacacatgaaggggctcattgccctatgtacactggtagtagctcctcttttcgcatacatggtgaactccatggatgtttctgggctctcctggtgcacgaggattatggtgtcatgtggggtctcaggggctctgacctactaccttgtgttcatgctacgctcctggcattggccacggcaaagagaaccagcagccgctggcagcagcaaggacgatgtggtttcctccggcatgctcaagtttttggcaaatgctttactcataacggcggcattgctgctgctgctatggtatatggtttctgtccgacttggtctggagacagcattggttgatacactacgtcgaaatctgcaacaactc

SEQ ID NO: 31 - DNA, 1215 bp, Oryza sativa
atggagttagagctggtgtatctgtatccagtgaccttggtgccggcagtcgtcggccggttcagcatccttgcctcgccgttgctgagggaattcgaggaacacaacctgcaattcaccagcataatcccgctcttcttcttctactcctacatcagcaagttcctctccgacctgcagggtcaaccactgcctctagtcaagctctatgcccgggcgctgcaccggtttaccagctgcagcagcagcaaaagccgtgctagcttgaggatctacatggacactgccgtcgtcgtggtgctgtcctacctgtctctgctcaagatcaacctgagctacatctggctcgccgtcttccccaccctcacgctcgccttcatcgccgctctgttcaacgaggagctccgcggtgacaggagagccgccgttgctgaggaagacgatggcaggaggaggaaagatcactcgctcgagctcaaggcgatggcggttgtgccctactgggtgctatgcgcgatggggcagtttcagggaggtgacagctttgccgtctcccagttccttctcttcctcggctccacgctgggggcgctggcgctgatggcggcgcggctgtcgcagctcgccggagcagcaccgggcttagcgccggcgtcggagcttctccgcaaagccacgctcgtggtgctgctggtgacggcgcacgcggtggccgccgagctgctgggcgaggcgacggtggcgctgctctgcctgccggagctcgcgccggcgctcttctggttcacccttcacctcgacggtggcagctcgccggccgccgccatcgacgacatcaaatcacacaggaatgcgctctccgttctcgctgtcgccgtcgtcgcgtacctcgcgaccgccatgggtgagcgtggcctgtccgtcagcaccatcacgatggtgtcttgcggcgtctcagggcttctggtccgctgcgctgtgctcgtgctcggtcagtggccggggcaagctgagacggccggtagtactgccgcctctttgatggaagttgtccgggtgctcaagttttgggggaacactttgctggcagtggcagctatgttgctgctgcttgcgttgctgactgctttccggttatgccttcaagaaccaatccttccgacagttgccaaatgtttcagcgactatattgacaaggtcccgcaaatgttagtt

SEQ ID NO: 32 - DNA, 1062 bp, Zea mays
atggattccaagaagatgtatgtttgctccctgcctttgatgccatacgtcgtcttcggattcgtcggctcccaactgccgtcagctcccaactggcagtcctataaagtggatgatccgaatgcgccgagaatgattcttctcgttattttgtacatctacgtcatcttgctggctaaaattaagaacgacaggatctacatcgagacggcaactgccgtggtgatatcttacgtgtcgctacttcaaatcagctggagctacatctggctcgttcccttccccattgtcgcgatcggcttcatcgttgctctgtgcaaggagctcagtcacgaggacgaggccggtggatcacattcacaggagcagagcaatgaaggtgaaggcgaagctactacgtcttcatgggaaaaggtagaggggctggcggcagtaacggtggtgccgtactgggcgcttctcatcatgggcctcttccatcctgacaagttcgccgtctcccagttcctcctcttcttcagtttcatgctgggggcactgaccatgatgatgactcggctggtggagacggggcgggccattgcccaccgcggcatcgtgccggcgtccgagctgctttggaaagcctcgctcgtggtgctgctggtggcggtgcacgcagtggcggcagagctgctgggagagaacgcgttactcctgtgcatgccggaaatcctgccggtgctgctgtggctcagcgttcgccttgacactgctgacgacaggatcaagctggtcctggacagtaatctgctccccgcagcactacctgcagtcctcgccatgctagcgaagggcgagcccctgctctactggtgcagaaaggcgttggtgtcttgtgttgtttcggcgcttcttaccttctacgtttatttcatgctgtgccagtggccgggaggacaacgcgatcgcacgctcacaggttcctctttgcagcacgcgctcatccatctcaagtcttgggcaaaggttttgctcaaagcggtggctgtgctgctaatatttgtatgcattctttccaac

SEQ ID NO: 33 - DNA, 1242 bp, Sorghum bicolor
atggattcgaagaaggtgtatgtttggtccctacctttgacgccagaggtgatcctcagttttgtgagttcccaagtggcgttactgatgcagtcctataaagagcacgagctgtatgtcaccaccgttatcattccgctagttattttgtacatgaacgcgtgctcattcgtcaacctgggattgccattgccactgtctcctctcaacttcttgcctgctaagaaagtctggatctacatcgagacggcaatcgtcgtggtgatatcttacgtgtcactactgatagtcagctggagctacatctggctcgttgtcttccccatcatcgccatcggcttcatcgtcgcgctgtgtagtgagctcagtcatcaggatcaggccggcggatcacattcacaggagcagagcaacgaaggtgaaggtgatgaagctactgcacctgtggggaaaaaggcagaggggctggaggcagtagcactggtgccgtactgggcgctttgcatgatgggccactttcacgcagacaaattcaccgtctcccagttccttctcttcttcagtttcatgctcggggcactcaccatgatgatgactcgcctggtggggctggccacggtccatcgtggcatcgcgccggcgtccgagctgctccggaaggcctcgctcgtggtgctgctagtggcggtgcacgccatggccgcggagctgctgggagagaacgcgctgctcttctgcctgccggagatcgtgccagtgctgctgtggctcagcgttcgcctcgactgtcatgacggcgccatcatcactggtgccgacaggatcaagctgggcatggacagcagagtggtcaatctgctcggcgcggcggcagctgcagtgttggcggccatgctagcggcgggctacatggacgagtccgtgctctactggtgcggcaaggcgttggtgtcttgtgctgtttcagcccttctaaccttgtacattgttttcatgctgtgccagtggccaggaggacaacgcgatggcacgctagtcacagtcacagccgcctctttgttgcaggaggcaatccagcttctcaactcatgggcaaaagttttgctggcagcggtggctacgctgctagtattgacaactctagctgctttccaactagttctactgcaagaactggcggcctctgcaacagccgaccaaattcttcaacgactaggtcaaaaattcctccagtccataatt

SEQ ID NO: 34 - DNA, 1273 bp, Eragrostis tef
atggattcaaaaatggtgaatatgtcatcgcaaccttttgcaaaagagcctgtcatcagtttactgattatttccctagttacaccgctgatacaggccaaggaagacaaccaacagtatgtcaccaaaatagctccgatgcttttggggtgcttcattagcttgtatgtccaattttggtcctatttcaagcggttgacggagtactctactgacttcacaagctctaacaataactgcatatacatggaggcagccggttttgtggcaacgtcttatgtttccctacttttggctaactggaccaacatctggcttgccatcttccccatcatcgctgttgggttcatcctcgcattgtgctatgagcttcgtcatcagagatcaaatcaacctgacgatggcgagggtaatgcgagcagtagtggtggtgtacagggaggtcagaacgagattaagttagctgaggtgcacaaggcagcagcattggtgccgtactggttactctgcgttatgtggcagttccatactgcggaaaacttcgccgtctcccaattccttctcttcttcagctctatgctgggtgcactgacgctgatgctgaagcagctagcgctgaccgccggagctcctcctggcatcgctccagcttcggtattgctgcggaaggcctcacttgttgtgctgctggtgacggtgcacgccttttctgccgagctgctgggcgagaacgtcatgctcttcatcctcccagagattttgccggtaatcctgtggttcagcattagcctaaaccgtcgtgacagctcgtatattaccgccgacaagatcagagcccagagaagtgtgatcactgttcttggagcagcagtagcagctgtgtttgcattccttgcgttctccatggatgagtttgtgctctcctggtgcaccaaggctatggtctcttgtggcgtttccggacttctagtttaccacgttgtttttattctatgccaatggccggaactggatagcacggcaagttcctttgtggaggcagcgttcgacctactcaagctttgggcatacgttttgctgaacgcggcagccgggttgctggcgtttgcacttgtgggtgttgtcaggcttgggctgcataaacaattgctggctcagccgaaatgttaccgtcaggaaccacatatactagaaagagaccccgcggcatctgccgcgggcgctggcacaatcgggaaaataattggtacgcataagataattttagtagtat

SEQ ID NO: 35 - DNA, 2207 bp, Triticum aestivum cv. Hope
ggatcctgtccagatctcaaccacacagttttgagatggtacgtactaattcctctttatctcttcaacacagtctactgtaattcctttttgtttgacagccgaaagtagtagcggccgagatcaagaacaccactacagcatttttttgttgataatttggtttacgtttgttaacacatgggaagcagccatcactttgttgaacaatctcttagtatattatagatgctggcaaaaactaagtttgaaaacggaaaacatacacgatgctggtaactgccaaaactaagtcagttgatcccctgaatatagatttgttagatgtctagcttgaacaaagtaaacctacaaatatattgtgacctcaagaacttccttatcttatatgcataattatgcgataaccattgtattggagaaagtctgctttttgtccccgaactaaaaataatggttttgggttcttaaattgaaaaaccctgccaatttggctcttgctttatgaaaccctctcaattccgttgatggtgatatgacaagggttttacacacatgcaacccagtcaaaaaagtgaatttaatgaaactaaattatctgtgcatccaaagacaagaaactacagaaattactaaaataaaagaaactattatagtaatataggaaatacattaaaaaaatataaccaaagaagaaattttagaatcaacctggcaaactaggtaattagagcaaactttgaaataatttctgaacattaacttcaaaagaaacttttgattatcatttatgtatatgtactattttaggtattctcaaatttgctagtttcatagacaaataatgtttaaaaaaattcgaaatgttcggattgcagttttttctccaaaattcaaacaagcataaatatattatttagcttctcattttttaattttttatttaatattttaatatacttttcagtagattttttcactctaaacatatttttacttgttgctaattacttggccatgacgtcatgttgggggttctaacattgatttaggatggactattgaaccgttcccgtggattggcggaaggagtggcggttatgatatgcccaatgcttctcgcacttgctctaaacatagttgacctcaatgatgaagtgtacggacatggagtccccattgccttgattgccatggcaggctttactttgatcaccggcatttgctccttcatggtgtgctgcttcatacaagggttcccgagaagtagcgtgatcctggcaatcatttcgtctgcttgcctcatcatgctcgcctacttcattgcacacttcattatcttcaaatccgtttttatcacccttggagtcctgtgtggaattttgctcatgctgcgaaccgtctgctacttgtatgatagctcaaatggcaatgttccagaatacacggccataatgcaaagcgtactggacgaatcacatgagttcttgactggcgtcactggaattctttttctggggttccaaggtttgactctggaaggtgacgttcggacaataagtcgcatgggcggggaaccaatgggttacatcagcttcatcttcatcactctaggtgtgggatcaatgctcctcgcgatgaccccacctaaaagttttggagagagaaaagttgtgcgtttgactctcctccttgactacttcatggcggctggtatcttcctgctattgttggttaccatgtgtaagcacacgcagctggagatggttgctctgttggtctttgtgcctccgattatatgcttcagttcacttctgttcctggtcttgttcccggtggacggacaagatgatgaagaccccaagccagcgtcactggaactgacaaaggtcatgttcaccggattcttggccgtttcaataagaaccatcagcaacacttcaccgaacaagctcaccggattcttcttactattatcctccatggctattgggtttggactctcatggaggctcctttcacagaacaacacaaagagtgttctcactactattgatgtttcacctgttcatgttgcttctgccgccaagattgcttccatttttacccacttttgtattgtaatcaccacaattctacttgtcgcaatggctagtactgccagcggtaaatgatcgactccagttggcaacaaaactactggtacc

SEQ ID NO: 36 - DNA, 2207 bp, Triticum aestivum cv Hope
ggatcctgtccagatctcaaccacacagttttgagatggtacgtactaattcctctttatctcttcaacacagtctactgtaattcctttttgtttgacagccgaaagtagtagcggccgagatcaagaacaccactacagcatttttttgttgataatttggtttacgtttgttaacacatgggaagcagccatcactttgttgaacaatctcttagtatattatagatgctggcaaaaactaagtttgaaaacggaaaacatacacgatgctggtaactgccaaaactaagtcagttgatcccctgaatatagatttgttagatgtctagcttgaacaaagtaaacctacaaatatattgtgacctcaagaacttccttatcttatatgcataattatgcgataaccattgtattggagaaagtctgctttttgtccccgaactaaaaataatggttttgggttcttaaattgaaaaaccctgccaatttggctcttgctttatgaaaccctctcaattccgttgatggtgatatgacaagggttttacacacatgcaacccagtcaaaaaagtgaatttaatgaaactaaattatctgtgcatccaaagacaagaaactacagaaattactaaaataaaagaaactattatagtaatataggaaatacattaaaaaaatataaccaaagaagaaattttagaatcaacctggcaaactaggtaattagagcaaactttgaaataatttctgaacattaacttcaaaagaaacttttgattatcatttatgtatatgtactattttaggtattctcaaatttgctagtttcatagacaaataatgtttaaaaaaattcgaaatgttcggattgcagttttttctccaaaattcaaacaagcataaatatattatttagcttctcattttttaattttttatttaatattttaatatacttttcagtagattttttcactctaaacatatttttacttgttgctaattacttggccatgacgtcatgttgggggttctaacattgatttaggttggactattgaaccgttcccgtggattggcggaaggagtggcggttatgatatgcccaatgcttctcgcacttgctctaaacatagttgacctcaatgatgaagtgtacggacatggagtccccattgccttgattgccatggcaggctttactttgatcaccggcatttgctccttcatggtgtgctgcttcatacaagggttcccgagaagtagcgtgatcctggcaatcatttcgtctgcttgcctcatcatgctcgcctacttcattgcacacttcattatcttcaaatccgtttttatcacccttggagtcctgtgtggaattttgctcatgctgcgaaccgtctgctacttgtatgatagctcaaatggcaatgttccagaatacacggccataatgcaaagcgtactggacgaatcacatgagttcttgactggcgtcactggaattctttttctggggttccaaggtttgactctggaaggtgacgttcggacaataagtcgcatgggcggggaaccaatgggttacatcagcttcatcttcatcactctaggtgtgggatcaatgctcctcgcgatgaccccacctaaaagttttggagagagaaaagttgtgcgtttgactctcctccttgactacttcatggcggctggtatcttcctgctattgttggttaccatgtgtaagcacacgcagctggagatggttgctctgttggtctttgtgcctccgattatatgcttcagttcacttctgttcctggtcttgttcccggtggacggacaagatgatgaagaccccaagccagcgtcactggaactgacaaaggtcatgttcaccggattcttggccgtttcaataagaaccatcagcaacacttcaccgaacaagctcaccggattcttcttactattatcctccatggctattgggtttggactctcatggaggctcctttcacagaacaacacaaagagtgttctcactactattgatgtttcacctgttcatgttgcttctgccgccaagattgcttccatttttacccacttttgtattgtaatcaccacaattctacttgtcgcaatggctagtactgccagcggtaaatgatcgactccagttggcaacaaaactactggtacc

SEQ ID NO: 37 - DNA, 2953 bp, Triticum aestivum cv. Hope
acgaaggccaattgattgactggcggccatcttggaaaacatctgcccgcaaagtgtatatataagaatcaatggagtggagccaaggatctcagcagggaaaaatattgtcagacgctcagacacctccagttccttcaggattgccattggttggtaagtctatctcgactctcttttgatgtccaagttggtaatacatccagtatcttgtatgcacgacggagttttattttcaaataaatctgagtgtgtcgtccaacctactgctcgattaggttttgcttgccttcctgaaggtgttgcttttgaagaacctttatcattgtccttgtattgtcaagagatggttggtgtcgatggtcactgttgtatatcatcaatcatggtttcctttgattcgggttttttttctccacctaggcatagcttcggtcttatatgactttgctctctgccggggtgatcattttatgttggctgtgtgcatggtagttatgtagaggccaggtgtatgctcattatgattgtatcgctcgatgctacattatgagtcaataaaagcgtcctttatcaaaaaaaaacttactgctggatcagggtatcgctagtctctccatacaatgacgaccatcaatgttgctggccatattacgtatgatagaatacataaatatgcacaaatcatttaaattgattaaatttttggagtgcccagaattcagggcatgtgaatctttaaatgattttcaagaaagttgtgtactttcatttgtataattacattcatgtatataaatgtacagttagatacttgaaagttctaaaagataaattgaactatattccctctgtacggtgaagagtgtacatctaaaaattttaggacatatcatggagttatgtaaaaaatgcattggaaaggtgcaagccatcatctctctcctctttaattactcaacccccaatgagctaagtgcatgtagcaattaagaagatgatgtgtagattgctattagtcttgattaccgtgtaatgagagagaagtattttttctactttaaaatgcattggaaagatagaagtacactctttgtggacaaattttgaagctagatgtacacttttcactggacagagggagtactggctatcaattcatgattttaaagtcttggagccaggctcataggtctgatcggcacatttttcctgtttcaggcaagggtgaaaaatcggatgtaacgtcgttatgcataatagcattcaggttcacactgttgagcagtgttggcaatttactagtggtatttcattatgatggtaccagcaaagacacaagtgagctgctaactggcacaattgaggttgctgcttacttctattatttgagcacattagcgccttcgaacgtgcgccccaacgcacggaatttcatagacacaggcgctcttgttattatgtcttatctattattactggacatcaacttgagctttttctggctcgccatcttgcccaccatggccattgttttcataacagcgctttacatcaagctcaatcatcatactggtgatagccatggaaaagttactccaggatcttctaacatcgacgagaagaccatcaagacaccgatggagcttcggatcatagtggtggcaactttcggggctctcctagtcatggatcagctcgacgacgatacagctggtgggttcaccatctcccagttcctcctattcctgagctccacggtggcggcactgacgtgtatggtgatgaagctgcctgccgaccgttttttgggcagtgctccggcgtcagagttgctccaaaagaccttgcttctgctcctactggtgacggtgcacacattggccgcggagttgttgggcgaggacgtggttctgttctgcatgccagaggtcatcccggtgctcctctggtttagccttaacctcgaccgcccgcaccacagcccgctcaccaacatctacaaaatgaagcgacacctgaagggactcactattgccctaggtgtactggtggtagttcctcttttcgcgtacttagtgagctccatggatgtatctgggctctcgtactgcacgacgatcatggtggcatgtggcatctcaggggttctgacctactacattgtattcatgctaaactactggcattggccgtggcagaagcaacaagcagcagctggcagcagcagcaaggacgatgtgccttcctcatgtatgctcaagttttgggcagatgttttactgatagtggcggcagtgctcctgctgctaaggtatatggttgatgtccggcttgatctccaacaaccactatagttggtagactatgtcgaaatttgcgatgactatgctagtatagtcctagtactccctacttgtgtatgtatgttgttattttttcaccatatatacatcccaattatgagcatggtagctacctaagtaaaagtacattacatgtcctagtgtactggaaatctaagtgtcttgttgggtttattatatcttttgtactatgtgttagctttatcaatttgaacggaagaagtatatgttaatgacatctaatctgaaacaagatatctctgatgctaatgctctaattgctatgatggatactattgaaaatttcacggatttgacaatagttgaatttactctgagaaaggacctgagaaaccacttgacctctctgctcaaacagcaaaaggtctatttgcagagaaggggtaaaattaaatgggtcaccatcggtagtgaaaatttagagtttttctcattatgtggctactgtgcaacataggaataacagtattgcctccttaattaataatgttggagaagaaaaatatcacacgaggacaaaacaactttgttattagctacatatgaacacccaagatcattttgcc

SEQ ID NO: 38 - DNA, 25 bp, Artificial Sequence
  Oligonucleotide primer
cattcgtgcgtaagcatggactaat

SEQ ID NO: 39 - DNA, 25 bp, Artificial Sequence
  Oligonucleotide primer
cattcgtgcgtaagcatggactaaa

SEQ ID NO: 40 - DNA, 29 bp, Artificial Sequence
  Oligonucleotide primer
atgcattcgtacatttttctgccactgat

SEQ ID NO: 41 - DNA, 18 bp, Artificial Sequence
  Oligonucleotide primer
gaaccgttcccgtggatt

SEQ ID NO: 42 - DNA, 24 bp, Artificial Sequence
  Oligonucleotide primer
cggatttgaagataatgaagtgtg

SEQ ID NO: 43 - DNA, 21 bp, Artificial Sequence
  Oligonucleotide primer
gggaacggttcaatagtccat

SEQ ID NO: 44 - DNA, 21 bp, Artificial Sequence
  Oligonucleotide primer
gggaacggttcaatagtccaa

SEQ ID NO: 45 - DNA, 22 bp, Artificial Sequence
  Oligonucleotide primer
catgacgtcatgttgggggttc

SEQ ID NO: 46 - DNA, 20 bp, Artificial Sequence
  Oligonucleotide primer
cctttttgcgacaagtcgag

SEQ ID NO: 47 - DNA, 21 bp, Artificial Sequence
  Oligonucleotide primer
tctctcaccatgtggctctga

SEQ ID NO: 48 - DNA, 20 bp, Artificial Sequence
  Oligonucleotide primer
cagatccgaaggcaaaacat

SEQ ID NO: 49 - DNA, 20 bp, Artificial Sequence
  Oligonucleotide primer
aagacatcaaacggcaggag

SEQ ID NO: 50 - DNA, 20 bp, Artificial Sequence
  Oligonucleotide primer
tactgatagtggcggcagtg

SEQ ID NO: 51 - DNA, 24 bp, Artificial Sequence
  Oligonucleotide primer
ggtagctaccatgctcataattgg

SEQ ID NO: 52 - DNA, 21 bp, Artificial Sequence
  Oligonucleotide primer
ttcccgagaagtagcgtgatc

SEQ ID NO: 53 - DNA, 25 bp, Artificial Sequence
  Oligonucleotide primer
tgagctatcatacaagtagcagacg

SEQ ID NO: 54 - DNA, 29 bp, Artificial Sequence
  Oligonucleotide primer
acaggatcctgtccagatctcaaccacac

SEQ ID NO: 55 - DNA, 29 bp, Artificial Sequence
  Oligonucleotide primer
tcaggtaccagtagttttgttgccaactg

SEQ ID NO: 56 - DNA, 21 bp, Artificial Sequence
  Oligonucleotide primer
gctagtactgccagcggtaaa

SEQ ID NO: 57 - DNA, 20 bp, Artificial Sequence
  Oligonucleotide primer
cattgccgaaaaatcctgtt

SEQ ID NO: 58 - DNA, 18 bp, Artificial Sequence
  Oligonucleotide primer
gaaccgttcccgtggatt

SEQ ID NO: 59 - DNA, 26 bp, Artificial Sequence
  Oligonucleotide primer
gattgccaggatcacgctacttctcg

SEQ ID NO: 60 - DNA, 23 bp, Artificial Sequence
  Oligonucleotide primer
gtgaaaaatcggatgtaacgtcg

SEQ ID NO: 61 - DNA, 23 bp, Artificial Sequence
  Oligonucleotide primer
cgttcgaaggcgctaatgtgctc

SEQ ID NO: 62 - DNA, 20 bp, Artificial Sequence
  Oligonucleotide primer
acgaaggccaattgattgac

SEQ ID NO: 63 - DNA, 20 bp, Artificial Sequence
  Oligonucleotide primer
ggcaaaatgatcttgggtgt

SEQ ID NO: 64 - DNA, 21 bp, Artificial Sequence
  Oligonucleotide primer
tctctcaccatgtggctctga

SEQ ID NO: 65 - DNA, 20 bp, Artificial Sequence
  Oligonucleotide primer
cagatccgaaggcaaaacat

SEQ ID NO: 66 - DNA, 44 bp, Artificial Sequence
  Oligonucleotide primer
gaaggtgaccaagttcatgctggaccctactctggaagacctaa

SEQ ID NO: 67 - DNA, 44 bp, Artificial Sequence
  Oligonucleotide primer
gaaggtcggagtcaacggattggaccctactctggaagacctag

SEQ ID NO: 68 - DNA, 23 bp, Artificial Sequence
  Oligonucleotide primer
acctcgtaggtcttccagagtag

SEQ ID NO: 69 - DNA, 20 bp, Artificial Sequence
  Oligonucleotide primer
gtgatacagggggcgcagag

SEQ ID NO: 70 - DNA, 21 bp, Artificial Sequence
  Oligonucleotide primer
gtggcttccgaaatagagtag

SEQ ID NO: 71 - DNA, 89514 bp, Triticum aestivum cv Hope
ctaataacatgactttcagaacaggattaccgtaccactctggtgcggaccctactctggaagacctacgaggttctgtagtaacttgatctgaagtttcatgatcatcatcattagcttcctcactaattggtgtaggaatcactagaactgatttcagtgatgaactattttccaattcgggagaaggtacaaattaccttatcaagctctactttcctcccactcacttctttcgagagaaactccttctctagaaaggatccattcttagcaacaaatgttttgccttcggatctgtgatagaaggtgtacccaacagtttcctttgggtattctatgaagacgcacttctccgatttgggttcgatcttatcaggttgaaacctttttcacataagcatcgcaactacaaactttaagaaacgacagcttaggtttattgctaaaccatagttcatacggtgtcgtctcaacggatttagatggtgccctatttaaagtgaatgcagctgtctttaatgcataaccccaaaacgatagtggtaaatcggtaagatacatcatagatcgcaccatattcaataaagtgcggttatgacgttcggacataccattacgctgtggtgttccatgtggcgtgaactatgaaactattccacattgtttaaaatgaaggccaaactcgtaactcaaatattcttctctacgatcagattgtagaaactttattttcttcactctaaaattctttgaacttttcaaatgtttcagacttgtgcttcattaagtagatatccgcatatctgctcaaatcatctgtgaaggtcagaaaataacgatacccgccacgagcatcaacactcattggaccacatacatcggtatgtattatttccaacaagttagtagctcgttccattgttccggaggacggagttttagtcatcttgccaaaaaggcacggttcgcaagcatcaaatgattcataaccaagtgattccgaaaatccatctttagggagtttcttcatgcgctttacaccgatatgacccaaacggcagtgcaacaaataagttgcactgtcattattaactttgcatcttttggcatcaacattatgaatatgtgtatcactatgatcaagattcaataaaccatcaccattgggtgtatgaccatagaaggttttattcatgtaaacagaataacaatttattctctatcttagatgaataatcatatcgcaataaacatgaccttatcatatttatgctcaatgcaaacaccaaataacatttatttaggtttaacactaatctcgaaaatatagggagtgtgtgatgatgatcatatcaatcttggaactacttccaacactcatcgtcacttccccttcaactagtctctatttattctgtaactactgtttcgagttactaatcacagcaaccgaacaagtatcaaatactcaggggatactataaacactagtaaggcacacatcaataacctgtatatcaaatatacccttgttcaatttgccatccttcttatccaccaaatattcagggcatttccgcttccagtgaccatttcctttgcagtgtaagcactcagtttcaggctatggtccagctttgggcttcttcgcaggagtgacaactttcttgtctttccacttgaagttccctttctttccctttgcccttctcttgaaactagcggtcttgtcaagcatcaacacttgatgctctttcttgatttctagcttcgtcgatttcatcatcacgaagagctcgggaatcgtttttgtcatcacttgcatactatagttcatcacgaagttctagtaacttagtgatggtgactagagaattctgtcaatcactatcttatctggaagattaactcccacttgattcaagcgattgaagtacccagacaatctgagcacatgctcactagttgagcgattctcctccatcttttagctatagaacttgttggagacttcatatctctcaattcgggtatttgcttgaaatattaacttcaataccggaacatcttatatggtccatgacgttcaaaacgtctttgaagtcccgattctaagacgttaagcatggtgcattaaactatcaagtagtcatcatattgagctagctagacgttcataacgtctgcatctgctcctgcaataggtctatcacctagcggtgcattaaggacataattcttctatgcagcaatgaggataaacctcacatcacggatccaatccgcatcattgctactaacatttctcaacacaattttctctaggaacatatcaaaataaacatatgaaagcaacaacgcgagctattgatctacaacataatttgcaaaatactaccaggactaagttcatgataaatttaagttcaattaatcatattacttaagaactcccacttagacagacatctctctagtcatctaagtgatcacgtgatccaaatcaactaaaccatgtccgatcatcacgtgacatggagtagtttcaatggtgaacatcactatgttgatcatatctactatatgattcacgtttgacctttcggtctccgtgttccgaggccatatctgttatatgctaggctcgtcaagtttaacctgagtattccgcgtgtgcaactgttttgcacccgttgtatttgaacgtagagcctatcacacccgatcatcacgtggtgtctcaacacgaagaactttcgcaacggtgcatacccggggagaacacttcttgataattttagtgagagatcatcttaaaatgctaccgtcaatcaaagaaagataagatgcataaaggataaacatcacatgtaatcaatataagtgatatgatatggccatcatcatcttgtgcttgtgatctccatcttccaagcaccatcgtgatcaccatcgtcactgacgcgacaccttgatctccatcgtagcatcgttattgttacgccatctattgcttctacgactatcgctaccgcttagtgataaagtaaagcaattacaaggcgtttgcatttcatacaataaagcgacaaccatatggctcctgccagttgccgataacttcggttacaaaacatgatcatctcatacaataaaatatagcatcacgtcttgaccatatcacatcacaacatgccctgcaaaaacaagttagacgtcctctactttgttgttgcaaattttacgtggctgctacgggcttagcaagaaccattcttacctacgcatcaaagccacaacgatagttcatcaagttagtgatgttttaaccttcgcaaggaccgggcgtagccacactcgattcagctaaagtgagagagacagacacccgccagccacctttaagcacgagtgctcgtaacggtgaaaccagtctcgcgtaagcgtacgcgtaatgtcggtctggccgcttcatctcacaataccgccgaaccaaagtatgacatgctggtaagcagtatgacttgtatcgcccacaactcacttgtgttctactcgtgcatatgacatctacacataaaacctggctctgatgccactgttggggaacatagtaatttcaaaattcctacgcacacgtaagatcatggtgatggcatagtaacgagaggggagactattttccacgtaccctcgtagaccgtaagcggaagcgttaagacaacgcggttgatgtagtcgtacgtcttcacgatccgaccgatccaagtaccgaacgtacgacacctccgagttcagcacacgttcagctcgatgatgatccccggactccgaatcagcaaagtgtcggggatgagttccgtcagcacgacggcgtggtgacgatgatgatgttctaccgacgcagggcttcgcctaagcaccgctacgatatgaccgaggtggaatatggtggagggggcaccgcacacggctaaggaacgatcacgaagatcaacttgtgtgtcatggggtgccccctgcccccatatataaaggagcaagggaggaggaggccggccctaggaaggggcgcaccaagtgtggagtcctactaggactccctagtcctagtaggattccacctcccatatggaataggaaaagaggaagggaaaaggagaaggaaggaagggggcgcccccttccctattccaattcggaccagaccaaggggaggggtgcggccacccttgaggcccttttccttctttcccgtatggcccaataaggcccaatacgtattcccgtaactctccggtactccgaaaaatacccgaatcactcggaacctttccgatgtccgaatatagtcatccaatatatcgatctttacgtctcgaccatttcgagaatcctcgtcatgtccccagtctcatccgggactccaaactccttcggtacatcaaaactcataaactcataataaaactgtcaacgaaaccttaagcctgcggaccctacgggttcgagaactatgtagacatgaccaagacacgtctccggtcaataaccaatagcggaaccggatgctcatattggctcccgcatattctatgaagatctttatcggtcagaccacataacaacatacgttgttccctttgtcatcggtatgttacttgcccgagattcgatcgtcggtatctcaatacctagttcaatctcgttaccggcaagtctatttactcgttgcgtaatacatcatcccgcaactaactcattagttgcaatgcttgcaaggcttaagtgatgtgcattaccgagaaggcccagagatacctctccgacaatcggagtgacaaatcctaatatcgaaatacgccaacccaacatgtacctttggagacacctgtagagctcctttataatcacccagttacgttgtgacgtttggtaacacataaagtattcctccagtaaacgggagttgcataatctcatagtcataggaacatgtataagtcatgaagaaagcaatagcaacatactaaacgatcaagggctaagctaacggaatgggtcaagtcaatcacatcattctcctaatgatgtgatcccgttaatcaaatgagaactcatgtctatggctaggaaacttaaccatctttgattcacgagctagtcaagtagaggcatactagtgacactatgtttgtctatgtattcacacatgtattatgtttccggttaatacaattatagcatgaataataaacatttatcatgaaataaggaaataaataataactttattattgcctctagggcatatttccttcagatccgcccccaccgtcgccgtcgtcgccggccacgcccctgccccggcccgccgtggtcgtcgccgacgccctcgccgccgccgtcgccctcgccggccgcccccgttgtgagccgccgccgtcgaggctctgtgttcagtgctttttttcatacatacatgtgtatttttttgttccttgcattttttcatatatataattgatgtatatatgtatgtggtagctatatgatgaatggatgtggctatgtgtgtatgaatataatgttcatagcattttgttgtttatatatatattttcatatatgtattaattttttatttaggttgttagtagatatcgattttaggttagtgcaatttaattaggttagtgtagaggaaaaagtaaaataaggaaaaggaagaaaagaggaagaaggaagaaggaagaagaagaataaaaagaatgagaggaaaaaggaaaataagaagaggaagaaaggagaagaagaggaaaggaagaagaggagaaataaataagaagaggaaaaaagaagaaaaagaagaggagaagaagaaaggaatagaggagaagaagaaaaatagatttctattttttcttcttctcctctttttttttatttttttctacttcttcttccttcttccttcttcctcttttcttccttttccttaattatttttctttctggagggagaagaagaaaaagaagaggagaagaagaaagaaaggaatagaggagaaagaagaaacttcaacacgaggggggtaccgataccccctccccgataacattattttcccatgtatatgtatgtcgcgtcgttgtcaatataaccccctcgagataacttcgacatgaggggcggtcgatatatataccccctctcgaccgtgataacttatacaacggcagcaccccctcgtccctcttgctcgaccaaaactctcgaggacacccaaaccctagagagaaaacgatgttggtctcctaccccctcccgccgcgcccctacccgacaaattaactctcttgaggccacccaaatttaccaagttaaaagagcgttgtcgtcgaggccaccccaaacccttgaagcgttgtgtcgaggcgaccccaaaccctagagaagcagcgtcgaggccactaacataattccttattgtgattagctagctagttctacgtttgccgctaatatatatatatatatatccatttgtactatatttgaataataattgacatgttgtaaatatttgcagaaactatagagcactcccgagacgagaagaaacagatgcgatgttgggggacataatcgcacaaggaagtgatgtcgttgcgtcatttctcaacgacaccgatggtcagaaaggactgggtgaagaagagggctatgttcatgatggctccaacccattaatgccggtacaagtagagggctatgttcatgatggctccggtgacccaatgaaggtacaagaaggaaaccgtgctgactgctccggtgaccgaaccgagtccggccggtatatatatatattagttaagcccgtgctgactagttaattgatgcattcattgttttcgtatatgtacacatattaattaactctcgtttttcttcctttttctagccctccggatcaagcacaactttgataaggagacgaggcccgaagaaaaatttgagctcgaattgaaaaatatacttcaaattcacaaactgttcatgacttacaaaatagtttaatgactcaaaaaatgttcgctaattcaaaaaatgattatgcatttcaaaaaaaaagttcgtgaattacaaaagttgtttcacgaatttccaaaaaatgttcgtcaactctagaaatgttcacctgttaaaggaaaatgtttctaaaatgttcacaatttttaatttttttttgcaaaccatctaaaatgttcatgaattcaaaattttgatgatattagaaaattttaaaaaatatgtaaaaatgttgatgaatttgaaaaatgttcatgattccaaatatgttcacgagttttaaaaaatgttcataattttcaaaagttattcacgatttcacgaaaatgacagtgatactgtatctcggtgatgcggagacatgtggtcatggccgtcggagattatgattttttttttctcccattacaatgcacgggccattttgctagtaataaataaagcatattgaatcatatggttagttaaacttattcgacattgctccctagctatacaattcagagaacaagaattagataaatttaaacaggtacaattcccttaatgtgaaaattctggtcatacaatttagagaatgagaattacacacttaagatactgaacgggcagtgacgaacgatctcacaccggagctgtgagcccaatagcacgcgtgctacatttagagcaacgcacaataggtgatcgatccagttatgcacactagtgatttctgttgagtaaataagtaattttttgattaatctaattcagaaataaatcatgaatatggcaataacaatattaatctcatgctgatatctagccatgcaaggtcgtattacgcacacagcagatatctacgtaaacatcaagcatgcatggtaaacacagaaataagcaggagagatagacaaattatacccgccggtcggcaaggtcattgtggcggtggtggcggcgactttggtctcgggagccttctttttggcttcggtggacatggtgatgtcaatggagatgaggacgtagtcaaagtaatggatgaagatgaacaatagcgagcagtagggtatgaaacgcttcccaaaaatgtaagcgcccatctcccggtgcaggatggcatggcggggttacggatgcctgcactcccataccgccgtacgtgcggtggacatgatgggattgccggaaggagcgtctgagagcgaaagaatggtgggtgtgtgagaggcattcatatagagcttttctcatgtttcagtacttgagtcataataacattcagttgaagtgtgccaacagaaagtgtattcttcatcattgtaaggtgttgcttctatacaaaagaaaaaggaaagaccaaataaaagatgcacagcggaggtgccaaaaaagtgaggatgagagaagaagcaacactattatcctttttagtatatgcaaaagtgcttcaaagtagcaccgctttcatgcattgtagagtacataggtttcatcagcctcacgttatatactagtagggaacctacattatagatcttttcttatatatttcgatgacgagtgtcctcgaacgatcccggtcttcatgagcaagcaagttggatgcaccctcaattagtcaataagaaagatttcatatattcatagctatgtgtccatcctagttgtacgatgaccactactcctagcgtcgacatcgatcataaggccttcctcattgcctaatggttaatcgttgatacaagactttttgttattaaaaccccaattaatactctattttccctttaagcgagacacaggcttgcgctcaactttgtaaaggtagttgagtggttgactaacacaaacgattttgctattgtacctgatgatctagttggacttgcattccggaaaaccacttgtgtatggtcttgccaccgccatggctggtgatttatactggcatcccgcatagtaccccatgccatgcacgccaagggagcaagagacgaattgttccgtgctcgactataccaaggcatagtgtacatgttgaagcctagcaagtggtaagtaatgtccacctaggcggcatagttgggatagatcataggagatccataatagaccacaatatagtatgccaaatagtcaaggtcgatcataatgtgtggcggaagagctgttgtcggagctcataatctgcaatgtaccatgcaatgaggtcagtggtgaagctggagacgccatcgatgtcagtcattgttgtagatccgatattgggcgatgaagacagcgaggttgaagtcggtgaatgcatgattgatgatgttggtgttgaagatgaagtcaatggtgaagacctaggcgatgtagatgacaatgtctatgaagatgaagctgatggtgaagatgtaagccatatagatgacgaagatggtgaaggtcatggtacctctgatccgatttggcggtgtgaggaaccccatggtcagcgccagctatcaagcgtgtttctgaagttcctaaaaatatgatgatagtcacacgtagatttgtttcgtagggggacataacttcaagttctagtgtgcgagtttaagaagaaattgtttaagaagggaaagagaaaatttgaagagaggaaaatgctagcaaattagtcaggttcgagtctactagtgtgcgagtttccagctgatccaaggtatgaaacttacaatgatagggcttaacttcttttatagaatgactcttcagatgtgcgagatagccatacatcttccaagcataattttggattttgggtatatcaagaccgaactgattattctattattaatgatgtggttgattaataatataatacgttgtgttcaccaactcagaaatagttgtgtttcccgtttggacccaatgaattgtcattgcagagcacatatcaccaaactctttggatgtaatatctgttttgctacacgcagagcactgggtaatgtagaagatttgacaacgtgttcaacatcagcgaaaagtttagttctaaaacaaatgaccattggcacaattcaaacatagggcatcaatgtgactatatcaaatgaaacaactggtgcgaaccaacctgtggttggatggttagaaggacagtggatgttggtgctcgcgttttcctgcatttatttcagaccttccgtcgatgtgcgtttactgagaggacacgctaccgttgactacgaaggcactgtgatgacttcgtcaatctcaagatgatgtgcccgcttagtctctcagaggtgtttatatgggtagggtgtgcgtgcatacgtgggtgagtgatgtaagctactttgattgtagtgtggtaaaaaaaaaagaaacaacttgcaactcttttgaaggagctctagagtgtttttgatgttggatctaatgagctagtgatgcatttcctgcaccgtaacgttttggttcatggtaaaccttagcatttttcaggctgggccgggccaggctgggttttaggctgtcaggctgggcaggaccgagcccaaccggaggtcccaaaaggcactcttttaagcccaaattacgaaaaaaatccaaagcctgacgtgaatgcttgttttcattatcacttatgtgttagagcccggcctgaaaaaccgaaaaataaaagctcaagcccaacacaaattttcttgtgtctctggcagcattccatcccaccacccgcgatagctatgtgtgatccatcctgattccagataggaatcgcctacgggcgacatagtagtggcccggggcattagctgcgtcgcgctcgctgcattcgatcgtttgcgctcgatcgattcggtctctttttctctttaggtttttctgttgtttttattcctttttctttgttttttgctggttttcttttatttccttcattatgttttctgttttatttgtttttgccatggttttctttggtttttctttttccttctttatactttgttttcctttgtttctttctatgattatttttgtttcatagtctttttttcatttttatcttttaaaacatgtatactttgtcatgcacattgtacatttttcgtatacatgtaaaacattcttttatacttatttaacattttcaaatacatgtttaagatttttacggaaacatcttttgaatactgtttttcaaatacgtattaaaacattttttccaatacatgttgaaacatttttttgaatgattaatttttttgaactgtgcaaacatttttctgaatcatacaattgcttatgaaaatatcataaatacattttttaaagtttatgaatatttccttatacatttttttgaatgtacgaaacatttttgaactacaaaaatagtttttttatattgtatcattttttaaatggaatttgtatttttttgaaatgtgtgaatttttttattgaccccaaaaaaattcatgcacataattaacatttttggtacagatgattaacattcagtcatttttcattcacaatatgcattttttttgtatggacccgaaatattttctatacatgtttaacatttttcaaagaaaatattataaaaattcaaatttgtatgaaagtgtataagttttttaggggtatgaaagtgtatatgtaatatatatactaccgtgatgctgcttgtggttactgggccgggctaggtcgggctccctccatgcgagccgtgcctaggcgttgcagtaagcggcacatagccgcgccctcccaccacccacgctcgtccgaaccttatcttctccccatcccctgcttctcctccggccccgtcactgcctcaccctgccgcattcccagagctgagccggccgccggcgccgcttgccgcccacaaaagtcttgccagaccacggccgcggtcttgccctctcctacgcagaagcgagccaccacggacgaacgtgccgtagccagccatggccgccgcctgccgtttcgccgcgccgctgggcctcgcgccgctgcctcgcggccgcgccgtcgccaagggcatcgctttatcggtaggctccaccaaggccgtcccacggggcgtcgcgacgaaggcggcaagcgggacgtccggccgcgaggagacgccggcgatcgtccgccccctctactgcactggcgtaggtacttggtgcctgctaattctgttagtaccaaagatatgggcttctgagcttaaatccggcattgaagcggtgcttgatacaaatactgtagtttcattgtactgacagaatcattctctgttgtcattaaaatttgaaattttataatttagatatatagaagaaagttatccctctgccttcaaattatcatgttgtggccttctggtctctctgaagaaacttatgtgttgcttgttactgtaatctttgttggctgagagttttcaagctggggaactgtaacccccctgtgccatctaattttgctaacaatacaacgagattgcgagcatattattttgttggtcgtggtgccaaacaaagtcattgttttcgtactattagtgaagtaggagttcttaagcacttagatgtagagtttctgccacttcaacataaactagactcaaccgtgcgcttgagctgattgtttgttcacacatgttgtgatctcttttattctcgcaccttttttggttacagatataattaatgaccttgtttcagttgcaaaaccaatctcatttcggacttgtttccttatttccaaggaaggcagatcatcaagagagcagaggaggtttttctttcaacacaagtaaaaggtaaaagttttagatctcgaatttttgttgtttcactgatgtttttactgatttatgtgtatatgatgtaccatcttcacatcagaaacttcgggtcgccatcctgataaggatctaaagggaaatatgaacttgctgcagcaacttggtatacttccttgtggtgctacggcatggttgagtttcgcacagtcagcacagtctagtgaaggagtaaagttaaatatggtttacgaggttggggagctgtttgagctgggaattcagctttcttatctgctcatactacttggtctgcttggaactggtacattttttgtcatccgccaggttcttgtccgcagggagcttgatctttctgcgaaagacttgcaggtatgcatcctgctaacatttctacatgcattcagactaccatgggtctatattatccctctgattttgttcttcagtttaatgtagagattatccctctgactttgttgttcaaacctgtcgacatgagttcttgataacatcgtactgtaattactgaatactctgaagtatagaacttcagtttaaggtagagattatcaatgatggcaacactgattgtgatgtacatgtaacaccacatttttactattgttattatgttaaatttttgactgtacattttcagtagtttgctgaatttgtcttgcttgatatagtttagttaggcaagagcaatatctaggcgagtgacgtgcgggcatgtaaagatatttttcaaagaaatttgtattgttctgaaattatctaacataaaaggtaaattctcctcatttagaaaatagttagtctcagtttctccttgaattgccagagttattgttgatgacctggctgacatccgacctctaccaaaggaacatcctaacatggcatctacagtatttgttctcaggatcttacattatttagtatgactaattcttgtggtttcctcaagcctaagtcatttttgttacttccatggaacgtatgggcattcaactagtcttaaaaaaaatatgatcttgaagtctggacatccctttcttgtggtcaatctttccaattttttgtttcagaaatgagaaagcacttggttagcgctgccttgttgtattcattacatgcgcaaatccttatatcatggtagcacaggcccaatccttgtactgacaagttaatttgtcttcatcgaagttctttgctgatgttgctcgcttcataatgaacgttgtattcgcacgctatgtcttgtatggtgtagatgaatgtggtgtacttacatcaaacatacttaattatcttaattataggaacaagtgagaagtggtgagggaagcgctactgaactatttgagcttggagcagtcatgctccggagaaaattttaccctgcagctatcaaatatctacagcaagcaatccaaaaatgggatagagatgagcaagatcttgcacaggttagatatccacaacaactagctagtctccctccgttcacaaatataagatgttttggatatttcaatatggactacatacggactgaaatgagtgaacagacacagtaaaactaacctatatacatccgattcagcattttcttagagcatcttatatttgtgagcggaaggagtatttgttttctgcattctaaaatctgtccgcaggaaatttaccttttaccaatgcaatgacatcatgcattgctgctagcctacataatacacgctgttcttagaaacagtgttgctggtaattaggtatcatgttcatttagaaactttttccttcttttaaccaaaggcgcagtttgccgttcaagaactactgaaatttacctgtgttttattatactgttttgtgaaagttgacatagattcttgctgtaagtatctgctattgaacctatatatatgtgtgcttttgggctgtgtgccttatccactaaaaaccaatacggccacccatgtcctgtctcttctctctgaaacagttttaccgtgatgaccaggtgtacaatgccctgggggtgagctacaagcgggagaacaaactggacaaggcaatcaagcaattcgagaaggccgtggagctccaaccaggctatgtgacggcatggaacaacctcggcgatgcatatgagcagcagaaggacctcgcgtcagcgctcagggcgttcgaggaggtattgctctttgatccgaacaacaaggtggcaaggccgcgccgagacgacctcaagtcgcgtgtgggcatgtacaagggggtgccggtgaagacgacggacaagcgatagccgccacgggtggacgaattcgtgcctcagttttggacagtagcatttgtaaacatgatgagaagttgtatgtagcatattggcggcgtcatccgtccgatctggccattgtagaatgtttcgaatattagcgcaaaaaaaaagtagaatttgcttttgaggtccgctccaagcccataacagctattttttgcgcttgctaacaaataaattatttctatcgatagtgtatcagcttatgcaccacaacggtgaggtggttaagttttgcccaattttaaagccccttcatagcccttattatttttttgaaggtaggctagctcaagaagaatcaccatttgagtcaacgaagttatcaaccggtcaggaattacaagatacagaatcctgagacccagagcttgcaacacaataaacacccaaaaactgaaaacagcagcttaacttgcagatggtgccggcctccccacgccctaaggagcaatcaggagtagatgctaccagagcgagcacgcatgtgaatctgtgatagcatcaggagtacagaagaccaaccacgagggaaccaatagagaatttagcccttgcccttcggcggcaaacacccccacagatgagatgggagagcctcctgacggctgcgtggacttgtgtggaatgaggatggtaatcccggcgggtagtgttggagctcttcatataggaggcctcctcgggtctccttcgttgatgtagccttctctctttcatgagtcaaatccacacccaaatgatgtcttgactgccaaggatcccgtaggaatgacccgggacgaaatcggagcaagatcaggaaagttctggccagtttaggaagcagtgtggaggtcatatgaccatccatacgagcggctcgtatggaggccatctttctcaatggacttcgggagctgttccgtattttgatcgcatttctttatacgaactccaattttgatgttcttgggctcgttggattgctagcaaaaaacccgatgcgtcagaggccttgtatcctcattttggacactttgataaatatcaaatcatcaaactttccaaagggccttgatctagtgtctgaaacttctccggtttgattgatgtgccaagtccatgaacatgccaatacacctacaaggaccaaaaaagaaggaaaactaataaaaacaaaaaaaaaaagttttgcaatgaatttggtaaaacgagtaaatacccaaaactatgcattggggacatgtatgagtgctggatctgtccggtgggagatggtagcgcgggatatcttcaaccgttttgaatggcggtcatgtaataggataggcgcttagtttacctacctctctttattatgccagccggttgtggcttttgggccttgttttattttgctagctctttgtgagccagtatcctttgttgcagcactttgcaagacattgttgaactactttatttatttataaatgtggccgtatgcatcgttctgatgcagaggccggggaatcccttttcgaaaaaaaacatgtatgagtgctagatggatatgataggaggcattttggcactaaaatatactctaaaaagtatgcatagggtttcgcagtggctgaattttgggtactatgtaccaatatgttgtctctttgaagctgttatgatccattccgtttatttaccacatacacaccatttcaagagaaagttgaataatattcgatttagtgttaccattatgtatggtaacaaactcatagacaaaatccgagccaaaactttggtggatcgttcactggccagcggagttctcagaataacatcaagaaaggtgatgaagccaaatttacatacgtacactagcgctaagctgaagcaccatgcaaaaatgcaaacacatattttggtatgatcttattcaactttcaactaagttcctctggacaattttatccgtacgggctatcgagtctgttgtatcaaataccatggggttgagctactccataactcttaccaagatcttcatctctccataaaaagtgcctcctatatttattcaactatacaataatttctttgtaaagttacatgaaactcatgatgaaatgtgggcacagaagacaaaattgcattcaccacaagtatcctaccatcataatgtaggtattgggagcaagcattcaatctcttctgaatcctttcaatgacataaagtaagaattccatccttaatttagtaacaccttaaagggatacccaaataggtgactggaaattacacaatttgacatcctaaaataacagaaagcacatgttcttcactatcaaatactacctccgtccgggattattagtcccctgagcgcctgagtctcgtccgtaaatattagactgtgatttgttttccaaggcaaaattaatgacgcttcgtctctcctgttggccaagcacggtgacgcagccgcgatctacgagccggcgaggccgcatgcacgcgtgcgcgaggccagccaatgcatgcacggtagttattgccaagtgcatgccaccaattaaatctattgccaaagaagtatccagccaattactatcgagcacgcactagtagaaaacagggctatcgttcgggtctggccagcccattagtcccggttcttcaagaactgggacccatgggggtataagacccggttcgtgagcccggggggccagccggggcctcgtgggcattggtcccggttcgtatggaaccatttgtcccggtttcacgcacgaatcgggaccaatggtcctcgctccggcccacaaccattggtcccggttcgtggcttgaaccgggacagaaggggctttagtcccggttcatgccaagaaccgggacaaataacttgcctatatatacccaccaccgcggcagagcactccacacagtgctctgttttttcaattcggcgagcagagggcatttgggtgctctagttcacctcctatgcacatgaggtattcgatgaaatgcccgagccacactagttaagctttctcctctcgaagctcgacctcggagctccattttttctgagatttgtctagatttagcggtctgtcacgccccgtccccgttttcaccaccgttgatcgcccccgccgatctcgtcgccagcaccaccgtggtgagcctcttgttcttaccttctttctgatacttgtctgattttcttactttagatagatatttgtctgattttcttacttttgacacacataattatatataatgcacgcagatgaaccggcaatggatgtatggtgacagacacacctccgagtacattaagggcgtgcataattttctcgaagtggctgaggcaaacaagttgaatggttttatgtgttgtccatgcactaaatgtgggaatacgaagtcttactctgaccggaaaatccttcacacccacctgctttacaagggtttcatgccacactataatgtttggatgagacacggagaaataggggttatgatggaagacggcgaagaagaagaggacgatgacaactatgtgccccctgaatacagtgatgctgcaatagggggagctgctgaagatcaagaggaaccagacgacgtgcccaatgatgctgcaacggggaagctgctgaagatcaagaggaaccagacgatgtgcccgatgataatctctgccgggtcattatcggtgcaaggacgcaatgcgaaagtcaaaaggagaagctgaaattcgatcgcatgttagaggatcacaaaaaagggtgtaccccaattgcgaagatggcaacacaaagctaggtaccgtactggaattgctgcaatggaaggcagagaatgttgtgcctgacaaaggatttgagaagctactgaaaatattgaagaagaagcttccaaaggataatgaattgcccgacagtacgtacgcagcaaagcaggtcgtatgccctctcggattggaggtgcagaagatacatgcatgccctaatgactgcatcctctaccacggtgcgtacgaggatctgaacgcatgcccggtatgcggtgcattgcggtataagatcagacgagatgacactggtgatgttgacagcgagccccgcaggaagagagttcctgcgaaggtgatgtggtatgctcctataataccacggttgaaacgactgttcagaaacaaagagcatgccaagttgatccgatgacacagtgaggaccgtaagaaagacgggaagttgagagcacccgctgacgggtcgcagtggagaaaaatcaagagagagtactgggctgagtttgcacgtgacccaaggaacgtatggtttggtttaagcacggatggcattaatcctttcggggagcagagcagcaatcacaacacttggcccgagactctatgtatgtataaccttcctccttggatgtgcatgaagcggaagttcattatgatgtcagttctcatccaaggccctaagcaacccggtaacaacattgatgtgtacctaaggctattagttgaagaacttttacagctgtggaatggaaacggtgtacgtgttgggatgatcacagacaggaggaatttaacctgcacgcgttgctgtttgtaaccatcaacgattggcccgctctcagtaacctttcaggatagacaaacaagggataccacgcatgcatgcactgtttagctgacaccgaaagtatatacatgagaagctgcaggaagaatgtgtaccgggccatcgtcgatttcttccgaccaaccatcaatgtcgaaagaaaggcaaacatttcaaaggcgaggcagatcaccggaagaagcccgccatgcgtaccgatgatcatgtactttctatggtcaatgatttacacgtaatctttggaaagggtcccggcggactagctgttctgagtgacgttgagggacacgcacccatgtggaagaagaaatctatattttgggacctaccctactggaaagacctagaggtcccctcttcgatcgacgtgatgcacgtgacgaaaaacctttgcgtgaacttgctaggcttcttgggcgtgtatgtgaagacaaaagatacaactgaggcacgggaggacctgcaacgtttgcacgaaaaagacggcatgcctccaaagtagtatgaaggtcctgccagctacgctcttaccaaagaagagaaggaaatcttctttgaatgcctgcttagtatgaaggtcccgactggcttctcgtcgaatataaagggaataataaatatggtagagaaaaagtttcagaacctaagtctcatgactgccacgtgattatgacgcaactgcttccggttgcattgagggggcttctaccaaaaaacgtgcgattagccattgtgaagctatgtgcattcctcaatgcaatttctcagaaggtgatcgatccagaaatcataccaaggctaaggagtgatgtggtgcaatgtcttgtcagtgtcgagctggtgttcccaccatccttcttcaatatcatgacgcacgtccaagttcatctagttgacgagattgtcattctgggccccgtatttctacacaatatgtacccctttgagaggttcatgggagtcctaaataaatatgtccgtaaccgtgctaggccagaaggaagcatctccatgggccatcaaacagagaatgtcattgggttttgtgttgacttcattcggccttaagaagataggtctccctaaatcgcggtatgaggggagactgactggaaaaggcatgcttggaggggagacaataatatgcagggacggatattcttggtctcaagcacactacacagttctacagaactctaccttggtgaccccttatgtcgatgaacacaagaacagtctgcggtccaaacagccggagcagtgcgacgactggattacatgtgaacacatcaggacttccagcagttggttggaaacgcgtctcagaggtgaaaacactgtttgtgaggagctgtgctcgttgtccgggggaccatcttcgaatgtaatgatttggaaaggatacgagataaatgggaatacattttacacgatcgcccaagatcaaaagagcaccaaccaaaacagcggtgtccgctttgatgcagccaccgacaggggaaaggacacatattatggttacatagtggacatatgggaacttgactacggagtatattttaaggtccctttgtttaaatgcaactgggtcaatctgtcaggaggcggggtacaggtagacccacagtacggaatgacaacagtggatctgaataatcttgggtacattgacgaaccgtttgtcctagcctatgatgtggcacatgttatctatgtgaaggacatatctaccaaaccgaaaaaaagaaaaaataaggaagcgaatacatcatacgatgagccaaagcgccacatagttctttcaggaaaaagggacatcgtgggagtggagggcaagatagacatgtccgaagattatgaaaagtttcatgaaattcttcccttcaaagtcaaggctgacccaagcatcctgataaatgatgaagattatccatggttacggcgcaataaggaaaggacacaagcgaagaaaaagtgaagactttgtccacaactattatgatgataccatgccaactttcaaccttttcgtagttcatttgaaatgcctattgtaacagacaagtttccgtatgaaatcttgatacttcgaaagagattgtccgttttgtacatgaagtgcatccagtttttgccgtaacccactcaactttcttgcacatgctatgtggatgaaatgatgataccatgccaatttccaaccttttcagagttcatttgaaatgcttttcaatttcagggtcttatagctcaaaataattagtaaatgcatgaaaaataacaaatgaagtcagaaggggttgaaaattgatgatgtgcatggtgcattttgaacacacaaaagtcagtagtacaaataagtttaaaaaatgaaatccatgtgtaacagatgagtttctgtatgaaaccctgatacttcgaaagagattgtccattttgtatacgaagtgtatccagtttttcccataaccctctcaacattttagcacatgctatgtgggtgaaatgatgataccatgccaactttcaaccttttcagtgtacatttgaaatgctttacaatttcaaggtcttatagctcaaaaaaaaaccagtaaatgcttttataaaactctaatagcaaaaggaatcaactaaaaagcttttataaacctctagtattttttaaactaaaattatataaaatttatgcaactaattatcaaagtattttctgttgaaaacatgaaaagcagaaataattatcataaagaattttttgttagaaactttagtagcaaaaataattttcataaagatttttttgatagaaactaaaataacaaaaattgtttttgaatatactgataaaacgcagtaatattaaatagcaggaaaaagaatcactcaaaatatatttttatagtgaagttattcacaaactagtgattcacacaaatttcaaagaattaaaatttaaactattcaaatttggaaactaatggagtaacagaaagactataattattctgaactaaaagaaaaaagaataaaaaaataaagcaaaaatcaaaagaaaataagtaattcaacaaacaaaaaaactggaaaaaaataaaaaatgctgcctaatgggccacacggcctgcatacaactagaaacccatctgtacatgggccgggatgcaggcccgcaggcccaataggcccaacacggcagtgacaagggtgggcaactaatgcctgcatattagagaggagctcagcacctgagctgcaacgcaacttataaacaggtgctgagctctctcagctagcgaggtgggactaaacttcccaccgtaccgcactgtgcaaggcctttagtcccggttggtggcaccaaccgggactaaaaggtgcattggtcccggttggtggcaccaaccgggcctaaagggggcattggtaccggtttgtggcataccaatgcccccttaggccggttggtgccaccaaccgggaccaaaggccttctgcttcccgccctttgggctgctgaaaagaggcctttggtcccggctggtgccaccaaccgggactaaaggtcgcattagtcccggtttgtctcacgaaccgggaccaatgcgtcgtctatataactggcactcgtcaaaatttcgttcagttcgttcttccccgccccgacgccgccacgctgcccgtctccgcctcgccgtcgccgtcgtcaccccgctccatcccgcgccgcccagacgccgtcccgcgctgccccgacgccgtcgccgccccgcgacgccgccggccctgcctcgcccgtcgccgcgccgcacctcaccgtcgcccgtcaccacgccgcccttcacgcatcatcactgtcgccgtccccagcccgtcgccctcgccgcgcccctcacgccgtcgccatcccgtgaggttaattatatgaaatttactaatttaattagatgtgtagttaatttagatgtgtagtaatttagatgtgtagttaattgagttagattttgttagatttttttgtagttcatagatttttttagatgtgtagttcatagattttttttgtttgattgttttttttttcatattgtgtaatttgttcatattgtgttagattttttttgtgttaatagattttttgggtgatattattgttgaatatgcatgtttgtatgttcatatatatgcaaagatcgaatatgtcaattttttatgatttttctgttcataaaatttttatgatttttttctgttgtaattttgttcttagaattttaatgattttttgttcatagaattttctttgtcaatttatttatatgttcatattgtgtatgtataggaaaattgtgtatgatcaaagtcatttatgaatatgttcatatttagaagagaggaaaaaggaaaataagaagggaaagtgtttaatataattagttagtaaaataataaaactagttaaactagtttatttttagtaagtactactttattttatttatagtaagtgcttaatatatagttgaactagttgatttaataaactactttattttagtatagaagtagtttatttttagtaagtactactttatttatttatagtagtgcttagtagttgaactagttgatttaataaaaccactttattttactatatatagaagtagttcccgcatcgacgtcgacgatgcctatcccgcatcctcgtcgtcgactcggcggtggaggccagcttgataagggccatgtccgggactgggctccgccgggctggtattgcgaggtgctaccttccggggggcgtaggttggtgaggagccagcccgttgttgacccgatccttgtttggtggcggtcgcgtgggccagtgatgatgccgaggcttgcagacaccacggaggtggtacggcaccgtgtcagcgaggaggacgagcacgtacgtccgtcgctacatggttgcgttggagggcaggttcgacaatacctggcaggttcttcagggatctcactaaagttatgatcctgtgatggttccttatctttgggtgtccaccgcccgcgaagatacccgtcgggcgctacggttctagctgtgttagcgatgctatatgcatgatagtattcgaagagtattagtgataatattcgacgatgtacggacacaagagatgatgtacttttgcttataattgaatgcctgctaatttgattactactttattttacgatttggttttgcttattgaatgctcatattggaaaaatactcctactttgaatgctaaaattggagagcactatgcagaaatttaggagccccctccattatgcacgccgtcgtggggtagcttctccttcattcccgtgtgtgctaaacaatttggtgtaataatgcactcgggaatgaaaggaggagctaccatcaccgatagtcaacccgtgattaataataatgatctaatttagttttttagtacatatatttaattgtacaagtttaatatttgaattatgaacataggccgaaaatgtcgtactcggacgacgaaaactgaccgggggagtgcgattggtgccacgacgaccgaggtatgtgcgataggttccttgagctggacgaagatcggcgcttcagcattaagctcgaggagaccttcgatgttgaaacggtacgcaacgacgacaatagtttttttcataattaagcacgacttcaactattttaacgtgtatttttcatcttttccaatttgactagcttatcccatgctatgcaagacgctatgtcttggagaggatgggttttgaagaccatgaaagtatggaaaccaaaaaaatccacctaaggacccatcatggtgtggattttgaagtaaagctgtacaattctgagagtgtaacccattttggttgcaaaaattgggaagcactttgaaagatgtatggttttgatgagggtatgcttgtcaccatggatcttggtgatcctacaatcgagcaagacaatatggacatttgggtccttgttgatacacttccaattcttcccccatgtgagcttctcaaacatagttattaagtactttatattatttatttcaaaatagttgacatcttatttccattgacaacttattttcattcttcaaagaatgtgcggaagatggtagacaaaacccactacaccgatggctccgaattaacttataaggagaaaaatcatctgatcgcattttgtactgatcttgagaattacagtatctacaatcgaactcctcaacattatggtcaatacatgccactagtgcacgtgttgaactacggtaactaccatggagataccccggtaagatttttttactattacgacatccgtgcatctttttgcatacttctaaaactagtacatgattgctaactacgaagttattactatgtttttcaacagataatcacgaatgatagtgtgcctcatccgatgtatacgcacggtcgccttgatgttttgaacatacaaccaggtcgtcctacgaatctcaactgtccataccggatttctaaaagaagtggagacatgacaatcaaagaatggaaaaaatgtatggacagtcgtaaggagcttcttggaagcaaaaggaagcgaagcacaagaattggagataggatgatctccattcttcgtaatggagagtcagggtctatattgttttatgctattttaccttaagggtgtttaggtcctacctgctactggtgatcatgtgctaagaacaattatgtagggttgggttcgatgactatgaggatgatgatcgtatgacttgttattaataacaagtagaagttgtatgatgatgcatgattagtaggacttgttattatatatgatgatatgtatgatgcgagcatgaagagttattatatatcagcgggtgaaatgaacatagtaatagcgttggtaaaccaagcaggaagatataagagaggacacttctctctattagctagctaataacaacctaaattaacccccaaaacccctaaaccagccactttcaaaaaaaacctcagctccagccagctgctgacgcgtggaagccttttgatcccggtttatgtcaccaactgggaccaaaggcccccctgcctgggctgcccgcagtgggcacgtggaggcccatctgtccggttcgtgtaagaaccgggaccaaagggggagggcattagtaccgatcttttagtcccggttcttaaaccgggacaaaaggcccttacgaaccgggacaaatgccctgttttctactagtgacgtaaacacagccacctgaattgctgcaattaattcttaactaaacgcgtgcatgcagttaactatggttgtcttggtcttggagttttggtctgtgggactaataatcccggacggaggtagtacattaataggagtcatactggatttttcataattaacctttagtatagaaaccacattagattacattgatacgaatattcataaacccaagcaatgcaatcttgtatggttctggttttaatgaaaccgcatgggttaacatggatcaactccgtaaatattttttgcaacctattagccaacaatttagataggagcttaatagtgcaactgaaaagagagataagtctataatcatcgacacatgaaaccacacgctgccccaaccttttcgggaataagtgtaataaaagagctattgatgctttgcaatcataaaagccctcaatagaattgatcacataagtataaatatttgttaattgtggtcaaacacctcctaataaggtatgtattggagccatcaggataaggagatttgttggagggtgaaagtatcttcaaggaaggttaatcttgggtgtttagttagcaaatcaagaatttctggaggaatggcaaaatgatcttgggtgttcatatgtagctaataacaaagttgttttgtcctcgtgtgatatttttcttctccaacattattaattaaggaggcaatactgttattcctatgttgcacagtagccacataatgagaaaaactctaaattttcactaccgatggtgacccatttaattttaccccttctctgcaaatagaccttttgctgtttgagcagagaggtcaagtggtttctcaggtcctttctcagagtaaattcaactattgtcaaatccgtgaaattttcaatagtatccatcatagcaattagagcattagcatcagagatatcttgtttcagattagatgtcattaacatatacttcttccgttcaaattgataaagctaacacatagtacaaaagatataataaacccaacaagacacttagatttccagtacactaggacatgtaatgtacttttacttaggtagctaccatgctcataattgggatgtatatatggtgaaaaaataacaacatacatacacaagtagggagtactaggactatactagcatagtcatcgcaaatttcgacatagtctaccaactatagtggttgttggagatcaagccggacatcaaccatataccttagcagcaggagcactgccgccactatcagtaaaacatctgcccaaaacttgagcatacatgaggaaggcacatcgtccttgctgctgctgccagctgctgcttgttgcttctgccacggccaatgccagtagtttagcatgaatacaatgtagtaggtcagaacccctgagatgccacatgccaccatgatcgtcgtgcagtacgagagcccagatacatccatggagctcactaagtacgcgaaaagaggaactaccaccagtacacctagggcaatagtgagtcccttcaggtgtcgcttcattttgtagatgttggtgagcgggctgtggtgcgggcggtcgaggttaaggctaaaccagaggagcaccgggatgacctctggcatgcagaacagaaccacgtcctcgcccaacaactccgcggccaatgtgtgcaccgtcaccagtaggagcagaagcaaggtcttttggagcaactctgacgccggagcactgcccaaaaaacggtcggcaggcagcttcatcaccatacacgtcagtgccgccaccgtggagctcaggaataggaggaactgggagatggtgaacccaccagctgtatcgtcgtcgagctgatccatgactaggagagccccgaaagttgccaccactatgatccgaagctccatcggtgtcttgatggtcttctcgtcgatgttagaagatcctggagtaacttttccatggctatcaccagtatgatgattgagcttgatgtaaagcgctgttatgaaaacaatggccatggtgggcaagatggcgagccagaaaaagctcaagttgatgtccagtaataatagataagacataataacaagagcgcctgtgtctatgaaattccgtgcgttggggcgcacgttcgaaggcgctaatgtgctcaaataatagaagtaagcagcaacctcaattgtgccagttagcagctcacttgtgtctttgctggtaccatcataatgaaataccactagtaaattgccaacactgctcaacagtgtgaacctgaatgctattatgcataacgacgttacatccgatttttcacccttgcctgaaacaggaaaaatgtgccgatcagacctatgagcctggctccaagactttaaaatcatgaattgatagccagtactccctctgtccagtgaaaagtgtacatctagcttcaaaatttgtccacaaagagtgtacttctatctttccaatgcattttaaagtagaaaaaatacttctctctcattacacggtaatcaagactaatagcaatctacacatcatcttcttaattgctacatgcacttagctcattgggggttgagtaattaaagaggagagagatgatggcttgcacctttccaatgcattttttacataactccatgatatgtcctaaaatttttagatgtacactcttcaccgtacagagggaatatagttcaatttatcttttagaactttcaagtatctaactgtacatttatatacatgaatgtaattatacaaatgaaagtacacaactttcttgaaaatcatttaaagattcacatgccctgaattctgggcactccaaaaatttaatcaatttaaatgatttgtgcatatttatgtattctatcatacgtaatatggccagcaacattgatggtcgtcattgtatggagagactagcgataccctgatccagcagtaagtttttttttgataaaggacgcttttattgactcataatgtagcatcgagcgatacaatcataatgagcatacacctggcctctacataactaccatgcacacagccaacataaaatgatcaccccggcagagagcaaagtcatataagaccgaagctatgcctaggtggagaaaaaaaacccgaatcaaaggaaaccatgattgatgatatacaacagtgaccatcgacaccaaccatctcttgacaatacaaggacaatgataaaggttcttcaaaagcaacaccttcaggaaggcaagcaaaacctaatcgagcagtaggttggacgacacactcagatttatttgaaaataaaactccgtcgtgcatacaagatactggatgtattaccaacttggacatcaaaagagagtcgagatagacttaccaaccaatggcaatcctgaaggaactggaggtgtctgagcgtctgacaatatttttccctgctgagatccttggctccactccattgattcttatatatacactttgcgggcagatgttttccaagatggccgccagtcaatcaattggccttcgtgcaggtgcagctatgtacttggtcgatgaaatctaccactgcaacacaactgtatactcatcttttacgatatcatgcatatattataatgcatgaacaaatcttgctcgattgcaatgatgcatgtaaatttatttgataaagggatagaaactggtattgttcagtcttcagtgtagttttcttgattcttattaagagttcgagttaaataatgtttgttatttacacctaagaatcatattgattgatggaatgcatcaatgcattgttgatatttgttcggacagaggatacaactctattaccatctttgtgggaataaatgaggttgttaactcttgctaggtacaaatatattagttgaaaaatcttgaaaatttatattaaattgctaaaatttgttggtataaagttgtacgtacgatgtatatgtctgaaatttctcatattatttgtgaacaatactaaaggccagctctttcaataatttaagaaataagctgcccaatcccgaattctcccataactcacccctcccatttcgtagggcttctaaagtagaggcccagcaaatcagaggcacgtcttattattccagtaggagtgggggcggcctatttttcaagcagcaaatagaattggctctaagtagttttgccctttccgaactatgagggcaaaagttgggaagaattattaaaaattaaaacaatatcttacgtgtgaactatgaggtcttctttatgactgcaattaagattaatttacctaaaatataaaatctgtgaaagcattttcagtaataacattcttatgtcaatgctcatatacacccaacatgattttccagctgtgtcgatgtctgcggctgctaaaccacaaagcccttcctccatgtcggcgcccgaatgacctctttgtcctcacggactgttgcctcttatttattggtggttacgtaagatcttgctaggccagcagtttctcatcatccatgtcctcactgtgcgcaaggttcttgcttgcaaataccgcaccgacatgagggccaggaaaggattacgagttatctataaaaatattctgaatgtgttcactttttttcagaagttgcttaaaacatccggcctctgcatcattgtgaggaacgcaaccatttttattacttgttcagcagagtacaaaagtacagcaacaacgatggaaccattacaagagacgaaagacctttatttggatattttcatatcatatttatttgaaatattcttctattcaattttttctgaatgcttgaccatttaattcttcttttcgctcgtaggaccatttttttttaaaattaacctaagccagccccttttctttaaagacattttttgttacacttctgcttcggtagaccccctcttcgtaagaaagggcatgatggtaatatattaaatcatacctcaactacaagtattttgggtctgccaatatttttttctgattttaaatcgcaaaaaaagtgagcgtgactcaaaccggggacattctcgaacaaatatagccacgataaccaagcagccaccatgaactgttgtgtttacattatcgttcttgttcttttcttctgtcctctttctttgtttttttttctttttttttacctttttctccttttttttcaaatgcttttattcggtttttctttttctttttcctgttcctttttgttcttatgcaagttgtagaacgattttcgacaccttatgcaagttgcgacacgtccgacaatttatcgcccttttttgaccgagaaaactccaaaaaatataaagtttatagaaaaccaaaaaaccttggtatggtgccttgaatggttcatacggcaatgaaaaaaaatttaaggtcatctgacggatgtcgtcgaacaacatgctctcaaacggagtcttctacctaccctaacaccctacgttgaacatggtgattttttctacaggaatacaagaaatgttctcaccaattaaaaaaatagcttcaattttttggttcatactttgttgaatttttttgaaattccaaatttgttactgtttttgataaaatgttcaaaaataaaaaatgttcgtgttcaaaattgtgttttccctttttaaaaatttcgtgtttttgacattatgagcaattttgaaaatgttcgcgttttgaaaaaaaatgggcgtagtttcagaagttgttctgttccttcggaaaagtttgtattttcaaaaaataatgtcaaaaaactacaaacaattttcaaaatacaaacttttcaaaattgtttgtagttattttctagatccagaaaataatgtcaaaaaacgatctagaaaattggaagagagagcgctacaggtatccaggcatctgcctcttcataaagcatcagggcatgtacaacgcggacgcttagaacgagcgccaggattagattcccggccaattagcagttattagcagttaattggttgactcctgatgttctatgaggcgtcgatgcaacatcaggcgtcgaacgcatgcacgtctttctgcctgctctaagcgcctgattgcttaatttactccatgaacttagcgcctgccgttgtaagcctgggcgcctaggtctttttactgtttcataatctttatttttttctagtaagcgcctctgtaaacagctgccattgtagatgcccccaatgctaacgctgagactaggatttcattccctagcgcatctgtaagtatcaccttctacatggaccaaggataacagattgatgtttgttaggccaccttcatcgcaaggagtcttgcataggcgcttagggacaattttttccccgtttagcacctacgatagtgtctttggctgcaacgctaaaagttgctcgccgacgctgcatgagttaacaaacaacaatccccaagactcctcgctctccaacgctaatttctgaggataccagattttttaagactttagccttatgggtagcacccgccgctagtaaactcgggtgcaagaaaatatctttatgaggtgccggaagacaactaatgcaccaacgcaaagagaaatctaccatcgtgcttgctaatagcgtctgcattgtagatggccttatagaaggcaaagacggaggccaaaaacattataacgcaaaaccagttaagagaaacatttgacggtcgtgcttaagcgttgttcacttcatgaaggcgtggttgctgaaaaatcttctcgttgtccttgtggtgtcaaaagatgggtagtgtggatatggtcattgatgtagtttgtctatcgctcttttgatcgctttgtatttttttcttattcttgcttcggcatagtttttggtgttatatgactatgctatttgcctgggtgtttatgtgtgtgcatgtgttgatcttgtctgtgtacatccaaactatgcatagacctaggagtgcactcgttgtttttgtatccccttgatccatcattccgagtaaataaaatctaccctttgtcaaaatagacccagacacatacgggtgctaccgtgccaacttattatgccatctccaacgggacgctagcggaaataatccaagtaatttggatattgtgttatgtattttcaccccaaatttatattgtcctctttcgttaagcatctaaataagggtccttccaatggagagaagtcttagtgttctttctttctcccggaccatcttcgacatgcatgatcatcaattaaccccttcacatactctttgcttatataagtgaacacaattactattcatagtggggagtatcatatactagtatcatgcatatgatactagtggatgatactacatatgcatgagcagaagagctcgcccaggtaataaatggagtcaaataccaaaaggacagcacaagctagccagctagcacatagagcaagtaagagctagtgcaaagcacagaaaacagtacagccagcgtgcgtaccttgttagctgtcgatgcacgatggacatggaagtatatatggtagctaccttgctgcaagcgtcctgatatatagtcgatttgtgtactcggcatacacgccacgtgtcatatagcgattggaaaactattttatagaaggcagcacttaacttcctcggcttatccgaagcaccaccaccacacaacttttccaagatgccacaatagtccacaatttgtatgtcactgtacatacaattactgtttactaattttcgtttgaattatgccgtacgataagttctggcctgatgtcgactttcccttgtaggactggctgaagttgcctgtgctcttccccaccgatggatgccattggttgccaccacgccataatttgcaaactgagcaggatccacccccgccaagtctagtattgctggctgaattggcgtcctcgaggtcctgacctggagctagtatcactgccattgccacgtcgagcgaggatatttggctcatcatggcatgccaccaccatgatagtagacctgtttggtctcaatgggaagcacgggccatggcctgtagctagagtgctcaaaggtgggcaggggcggtctggtgcctgacccaatcctcgcgccgcaactgctcgtgggtgcatacaccgcaggagatgttggcctacatcctgcagagggggtagatgaaaagttccacgaacaaccctcacaagaaaagagagagccatggacaagctagcagaagactcagcaaggcaaaggtgttttggttccgggcactccttgacacatgttcatttttgccatcaaacttgattttgatttcatcctcgtttgatacgttgaatctctctttattggtactatatacacgttggatcttttgttaatttttatttccttccttgcattttctttttgttcatgagaatgagatggctaagatctggttggtgaatttctcacaaaagaagaagatctcgttggtgaaatctgcatgcatgcatatctcttatcgtttttcatgaagttctccaggttcatcgtcagtctgtccttgataatccaaatgatttcttagattggtcttctgtgttcactggctccatactaacgaaaataggaaatttctagtaatactttgttgtctagtcaagtgaaagtattacgaagaatattgtttttgaaatgccgccacggggggtggacactgcccaagcacctgatattttgaagaatatgcagaaggaacaaaatacatcaaaccagtgaaacatgtatcatgcagtgtaacttgttttaatatatgaaacctttgcgaaactttaatgaagtatacgtccggaacagtgcataccactcgaatgaaccatatcgagagaaaaccaagtttaagcgcagtgttatgcattttctaatcatccgtaatttgttatcatgcttcacaaattttattcatgtttcatatagttttggttgtgctttatgagaaaaccagaactacaaagccaatggagttgctaaattttggtatatgtatcacatgatgttttctaaatacatgtttcaggaaggattgcagacatgttccatcattttcctggacaaggttaaacctatgcagatacaataagctatcaagttttccttcagaactctcagcaacaaaaagttttccgtcggaatgtttctttataatgttttcaaatatttacaaagaatgttaattgtttttaagtaagataaaaagaatctaaacctcgtggataaaatgcaaccataagcaaaaaataaaaccaaggaggatgaatccctggcctatgtatcaatagatgcatacaatcattttttattaattattcaataaatacctgatagaaacatacatcagagaacacgaagccatcacccatacccaccaacttgacaatggggaatctcatcacgccccaccatctagaactggggtcattgacaatccattcagatgacgtaccaagggcatacacccggtgcaatagacccaaagtatacaaaaaatgtacatgctttataagccgccgccatcatccaaacactgacccatctctaggaaagagatctgcattatcattgtgaggctggccagctgtcgacgccctcttgagggaagacgaactcaaaagcggcaccatcatccgatctaggagacccatatcaagggttcccctcggaacaggccgagctggagatgatgattgcagtaattattccttgaataacgtaacaacgaataaacactgccatcgccccatgaccgaagtcggagcacggttttcaccggcggccacatcttcccatctagcagccagctagatcactaccaagccccgcataccggctctgcactaggccccaacagcgccaccgaatgaccatcggtcgtacagacaacggatgagacagtagcaccaccgccggcaggcaccaccacgccgctatcttatgccgtcgccaaaagtgaggcaccgcacggtggaggtttagctccgggggcacgttcctgcacgacagggtgccccgcaccacccatctgcgacgcaagaggaacgcctccgccactaagctcaggcggtttcatccagccgcggcggaggtgagggaaagaagcgtttttgggttcttcctgattgacgcgagctctgtgtgtgagttttttttgggggggggggggtaacgtggctaaccggggatttgcatctcgtgaaatatattacaatgaaataacagtttggcgaatcttttacaacggggaaggacttcaaaggaacatgcttgatattctggtatgttttctgaaaaacagtagccgatccagatctacaaaggagtggttcggggtctttgatctctccctctctctggtctggtctccctcacttttatttggtggccttaattctctatataatctagtgcagggcacgccagactggtctatagtgactgcaaaccacaaaaaagttacaattacgtatggacaaaagaaagtaaacaacatacttgtggaataagagaaccggtgagaagtagggtgggataaccgataagcctgagcaccgagttggatttccggggataacacgcggagtcgactggtgaggatatgacggagtagaccaggaaagacgaacgagtggtagccaagtattggaagcagctatggccccatctcgattccagagagttcccacctgatccacaacttgcaacgagtggcttggggcttactcgttttgtggtctatattgtctctccataatatctattccctctttcccataatgtaaaatgttttttgacacaacactagaggaagtatataccatcacatagcgttttaaaaaaaagaatctactccatccgttcctgaatatgacttgttttagagattacatatggactacatacggagcaaaatgagtaaatctagtctctaaaatatatccatatacatctgtttgtagtccgttctgaaatctcttaaaagacttatatttaggaacagagcgagtataaactaataaatgcaatcatatctactccctccatcctttaaagagcgtactttcaactttgttggagggtcaaactatctcaatgtttgaccgagtttgtgcaaaaatatatcaatgtttatgaaaccaaataggtatatgatgaaaataattttatcatgaatctaatactactaattcgatggcataaatgttggtgcattattgtataaatacggtcaaatataaaaaggtttgactttccaacaaagttggaagtacactcttcaaaggacggagggagtatgatacgacctttatgataatttgcattacaaaaataataacatagattactgtcatatgtctgatactttattcgttctgaagtacttgtcgtggttttagtttaaatttaatctaaaaccacgatagattttcgggaacggagggagtaatagtctaacttacttcctactatgactagctttgccatcacgaaagtgtcaatttggctggtgggacacgacaagcatcaagtcataaggtaatgttcgtgatcgtgcctattaatgtgttgcatgcctaataaattgcacactttcaaaccacaaaaatcatggaatcaaaataatcattatatatgaattacaaaatggaatcaaaatagtgttcacacggttacaaacatgtgggaaagcaacatgcatggaagaaggacataggtacaactgcaaccggccgggtcagaagtctttgggcgtgaaacggtatgcacaggagaccagcacgcatatccaagcaaattttatcagtcaaccatataattattccaagaaaattattcggaaagactatagacttatcactcaaccacaataatgtataagttattttcagaaaataaaatcaagagagactatgtattactactattcaaccaagttatcgtattagttatcccgaaaaactaaatcgaagaagcatgcgttgctgatttgtgatacgtggtttggaccataattcctttctgttgtttgaagctagtaacgtcaggctgttagatactacctccgtcctggtttattggtcctttttatagtttgtgccaaattttgactaaagatttaactaacaaaatgttaatgcatgtcaagtaagattatctcattggattcatatttgaacatagttttcaaaaatataatatttggcgacatgcgcgaacattttgctagtttaatctatggtcaaaatttgacacgaaatacaatggggaccaataaacccggacgcaggtagtattgtacaagaccttttggtggtgttcaggaaagctaggcaacccctactgtttctagaataacttgatgacccaatacgtccttgtcgcaaaaaccaatgacattcatgttgtgttattttacacccatcctttgtcaggctaggctagtccctttggtagcatcacatgaccgcatgtcgaccaagttaacaccaaaacggaccaaccaagcaacatgactcttgcctgaacagatggagaaccagccgacaacggacgacacgacaaaaatgttaagcacccgaaacagggaagacccgaaacagggaagcaatgacgcgccaccgatcgatagagtaggaggtgtgtaacgccccggtttcgttgcgccaggtgtctgccagttattcgccgtcgttgccatgtcatttgcttgcgtgttgcattttatcatgtcatcatgtgcatttcattttgcatacgtgttcgtcccatgcatccgagcattttccccgttgtccgttttgcaatccggcaatcctatgtcatccggcgtcctctttttgcctcttctcttgtgcggatgttaaactttctcggaatgacccgaggtttgccaagcggccttggtatagcaccgatagaccgcctgtcaagtttcgtgccatttggaggtcgtttgatactccaacggttaaccggttaaccgcaaaggcctcttttgttgcagcccaacacccctttcaaagtggcccaaaacccacctaactcccctccatgctctcggtcgttcgatcacgatcgtgtgggcaaaaaccgctcctcatttggactcttctagctccctctacctataaaaactagcccctcccccgaaattccggatcaaatcctaccctaaccatagcttcttcctcctccagccgccggacgcgtccgcactgcaccggacacgtccgcccgccgccgcccggccaaccaccgccacgtggcctcccgccaccgccccgcgccgccggcccgcggagcccatctggggcccgcacgggcccgatccgcgccgccgccgcccgagcgctcctccgcctgccccgccgccgttcgccgtctggccgcccgccgcccggccgcgtcgccgccgccggctgccgcctccgtccgccgcccgcgccggccgcggcctcctcctccctgccgccgcctccgccgccccggtgcgcctctaccgccggcgcctctgcgcccccgcgcccgagcccgtccgccgccccgagcctgcctctgcatcatttcatttcattccattgcatcattttcttttgagctcatcttgatgcccgaaatgctgttggaagagtgcatgttgatgttaatctgctgaaactgttataacttgctcatttgtcatttttgccattattgatgtgtgcatcctatgaggttgatgtctacatatgttttgatctatgctatgccatctttccagaggtgccaaccatgtatttttgtgagttgtgtgctgagtgcatcgagcttgttaagtagggcacttgctgttgctgttttcctggcagattctgttatattttgttgctatgtaaacatgcttctacaagtcatttcgtgcataatctggagatgttctataaacatgttttgatctacatgtcatgctctatccatccatgcccctggttgcatttatagcttgctgtagattattcatatcttgctccaaagttgcttgatgatgttgctgtcagcctgttaacattaagttcaatgtttccatgattgtttgctagtgatccatgtaccctatgaacttgctcttgccatgcttagattcacaaacatgtcttattactgttggctgccttgccatgccatgtattgctctgtggtgagtggttcaagctcacgaacaagcctacttattgttgttcctgccatgtatgaatctgtaatataacttgccatgtttacatgggtgccatcatattttatgatcctttttggctcatggtcagtaagggacttttgatctatgcttttagtagattcatgccatgcctttgtttgccatgatagcttcctgtaacatgttgtttgatagctctaaacattgcaacctgatgttatttcctgataagtcaaactgttataatttgcaatcttcccatgtcattttgagcatgttctaatagtttctggagatagctcagtgttcatgttttgttgtcctttacctgtacttcatgcccatgtcttttgttattatgttggggtgctgtagcatattgtttttatgcttgcaatatgcctacttgctgttatggacagattgttgctatgtcttatatagtgtatgtgttgaaccgttgctccgttttgagtgtgctctatatgaaacttgcttgattttgcatgtagtttcatattatcatgttgcatccttgttttgaggtgtttgcttgatgtttgtatgcattttgcatcaatgccatgtttaacttgttttgctcatatcttctaggctgtagctccgaatctaatgaactttatatgtaacttgactagaatctcgtgtagatcatcttggtgcatcttaacttgttgtttaacaacttgaacataaggtttattcagatctggaccaatttcgaaatttgcatatgaggacttaccggaattgttatatgttgttcccggcctcatttaaacttgccttgatgtgttgttcttgtttgcatcatctcttgccatgagtagcttcatgtagcttggtcatgcatcattcttgttgtgcatcatgccttgttcatgtgtggtgtgtttacctatgttgtgtgcttcttcttgttagttcctgtttcgttgcgatcgtgaggattcgttcgtctacggttggttcggcttcatccgttcgtcttcttcatggactcgttcttcttccttgcgggatttcaggcaagatgaccgctaccctagatctcactactatcgttgctatgctagttgctcgttctatcgctatgctgtgctacctattatctgtttatcaagccatcccatattaccatgaacctctaacctttgaaacctttcctatgcaaaccgttgtttggcatgttaccgcttttgctcagcccctcttatagcgttgctagttgcaggtgaagttgaagatttctccatggtggacaggattttggttgggatatcacaatatctcttatattattaatgcatctatatatttggtaatgggtggaaggctcagccttatgcctggtgttttgttccactcttgccgccctattttccgtcataccggtattatgttcccggattttgcgttcctaacgcggtcgggtgatttatgggaccccccccccgtgacagttcgctttgaataaaactcctccagcaaggcccaaccttggttttaccatttgcctcaccaccacctacttttcccttaggagtaatgaacccaagggtcatctttattatagcccccccgggccaatgcttgtctaagtgttggtccaaactagagcaccgtgcgggaccattccttggcaacttggattcgtcggtacctgtacgcttcgcttatccggtgttgccctgagaacgagatatgtgcagctcctatcgggattgtcggcgcatcgggcggctttgctggtcttgttttaccattgtcgaaatgtcttgtaaaccgggattccgagactgatcgggtcttcctgggagaaggtctattccttcgttgatcgcgagagcttgtcatgggctaagttgggacacccctgcagggtataaactttcgagagccatgcccgcggttatgtggcagaagggaatttgttaatgtccggttgtagataacttgacaccagatccgaattaaaacgcatcaaccgtgtgtgtagccgtgatggtctcttctcggcggagtccgggaagtgaacacggtttctgggttatgtttgacgtaagtaggtgttcaggatcacctcttgatcattgctagcttcacgaccgttcctttgtttctcttctcgctctcatttgcgtatgttagccaccatatatgcttagtcgctgctgcaacctcaccactttaccccttcctttcccattaagctttgctagtcttgatacccatggtaatgggattgcttagtcctcgtggctcacagattactacaacaacagttgcaggtacaggttatgcgatgatcttgacgcgagagcgatgcttgcttgcttgagttcatcttctgcttcttcttcgatcaggggataggttccaggtcggcagcctgggctagcagggtggatattgtttgagtttctgtttatgattcatccgtagtcggatgttgttcttgtatgttgtattgttgtattcgtgtggcattgtatgccttttgtatgtacccccatctattatgtaatgttgatgtaatgatatccaccttgcaaaagcgtttcaatatgcgggtctatccttggtgggaccttcgagttccttttggatagggtcgcatattgacgtacgtgacaaggtgtgccagacataacctaagtcggcgcacgccaacagccgaataggccgtagagctagccattggatagtccctggcacgtgagccaaaggtgtgcccgtggccacctcggctgctgcccaatacccttgccttttgtgctctgccctgagcattggtgctagggctcggtgtttcctcggcacatggaggcatcatgtctgcgtgcgacgggtgcgccctagatggaatgtggcatgtgcggcatacatgcgcagattacgcccatgatgtcgggcacaccatacatgattagaccaattgcagccaagaagtcaaataacttatgaacaatgtgttttataaatgacccgcaaggggttctgtttccatagaataagcacaaatacaactacactaagaagaatatgcgcaaccattttcgtatgctgtttcacgtgcgtgctatacctacctattttccatgacatgcggtttgcggactcgtgtagggacaccttcgtgtggtgtttgtggttgcccatgtatgcatggctcgtgcattggtcagcatgggccaaacgaggcggattcggcatatctgtgtcgttcgtttcgtgtagggctctttgtgttatgtgaggggggtacacgcaacttgtttgctagatcttgcatgctcggctttgccgtatggagttgtgaggggcttggctggtcggcctagtttgtgtgtgtgctcgaatgttctatgttactcggctgccatgtgccccccttggcctcgatgtgttgtgttgtttctttagttttgtgggggcatctgatgtttggtccaccgggtagaggcaatcatcttgcatggagcctgcctgacttttccttgcgtgtgccggacggctcgtggtatttcttggaccacagccagctatgccgcccaacacttggatgggtcttcagacagacacatttggacacactgcaagctgacatctttttggtcagataaggtgggctcattctattggtctgttcccgtatgtcttctgttggttcttgcatatccatctgtctagtgctagtgcggttgcagatcctacaacatgttcagagtgcatgcaacatgaagggcaaaaacctaccagcaatgtgtgatgtttgttgatttcagtggaaccgcacaaaaagactagaagttttatccaccatgagattccatgaatgttcaaactgataataaactcaatccattctacaagcatatgaatttgatttatggaacgggaaaacctcatcaaagtttgctcattgatgctatcaggtagcaattccacaagaatgaacaagagttaatcagtctgaaccccagcaaattaaagtttgcacatgtgttcgatcagagaagcgtggatcaatttttgtttatttgtacaccttctcacttattcggctctcatctgcaaataatcgagatacagaaacacgcacgttggacttatatacgggagtctaatgaggtggttgtgaatgttgcctacggctttgaaccagacctgtcagtttgcatgccactaatacaatgagccttcgaatatcaagttaatgctacagtcaaagtggtgaaataatcatatgatctaagtaaagtgcctacacaacacaaagattcatgattgtagttcccatggtgcggcaaaacataatcaccactagcagcgaccacacccacgattgcatttcccacacagtggaaaaacatacccactgccaccgccgaccataccccccagtgcagttccccacaggtgagaaaacataaacaccgacaccgcccactacacaccaattgtgatccatcatcttcgtctcccccactacaaactaagaacccctacatccaatgatgtctgagaagaagaagaggagtgaggcttttccaatggccaccctaggggtgaacatgcatgagaagttcgacacggagtttaccaaggcgttcaccatgcgggcccatgacatgaccatggtgaaggtgatgtacatcaacgagctagagtcggtgaagcgctacatgtacacattctagcggtacctacaggaggatgtgctttgctacacctacgtaaaattctaacgtagttttacgtgcgggctggcttggcacaactgaattggaagaaaaactcgaccgggccccaccccattaaaatcagggggagggtgattggttgagggaggaaagaacataagttcatgttaataacttgcgtagatgtagcattattgcaggaggataatcacaaggtggtgggactcaacctcgtgtacacatgtccacatccttgtacgaaccaacagatcattgttattcagcccaattacacttgattgccagactcccacggatgctctcattttacatgagtcatctaaatatagcatgtacgatcttacctgtctaatcaacaaatatctttatgtacaagtaaattatgtttgaagaatccaatgacgatgaggtcatattcttgtgtttactattcttacacaagcacacgagacaagatctcccgtttccatacgcacatccagctgggttggccctagcaggtccaccctactgctagggttggcatggcttgcttgcatggttgccgtcactattctgggtgtgagggccgtctctgttggtgccgcctcttctattcggggcattgccacctcgaccttgtcacttccttctctcccactcccactctgagagctggagacacccatcgccggatttctcacagccatcctcgaagcggtcctcgacaacccgcagccggcacgtgagctccttgatagagcgctcacgcaagtcaagaaatgtatcgatggagactggtacctgtgcaaagcgagatgggatgacgcgcagaaacttttgcatgatccgcatgtcatccatggagtcggcgagcgagtggacctacgtcacaaggctggagatttgcatggtgaagtcatagacaagctcaccttccttgaaggtgatgttatggaagtccacccacaacttatgcgtgtttgcctcatgcatgttgttgctgctgaggcgctgggttcggaacgctgcccacacctcattggtgcccgtctgcgccatgagcatgacatatatctccggcttggttgagtggatcaatgccactatgtcatgccgatcctgcttcctcgtgaccaagtcgtcatccatgctgacacggaggctcgccgcatggaggttgcattctatgatcaacgaccactcgttgtagttcatgcatgtcaacatgggaaaggtccggcacaccaccgctgcttgctatcttgtgcacaatctctattagcatgacattgccaatgaagccatgtcgggccccctgttgtgccctcgccactcccaaggaggcggtgacttggactcgttatgtgatggttcttatttggaactacccaagacgaagcattgctgctctccttcaacacattccatggctaccgaaatctggacactctggtaccaattgttagcttagatgaagctttcccggtcaccagagtggacattcccgtagagggaaaagtcagcttcgaagagagcgagccagagaaaaggcgagagagaaacagagaagttatattttggctgcaatctctccactccacattggagagttccattactcttatctatctgtacaaggcaatagaaacttactacatctgagctagcaacttcctagactatgaccactccatgacacaacatgcatgggagcagtggagtatcactttactctatgatgccacttatgtcatggtttttgaatagatgtgcgatatggggtcgccccatgtgtagtcaacacatggtcccccacctggggccaccccatgtgcagtttgccaaatggtgtttgcccggactgcccaaggagcttgccgatagacgggattgaatcaaacattctatagatggagatccctgaataagacaagatagggtattatcatcaagttgcccaacctttcagttcgttgaagcattatcattgtaccccaaaagttatgtaaattttagaaaataaattaaaaagatgtggttacctctgactggaaagctgcgattctaattggtcctaacgtaacatcaccatataggggaaccaacatataaagacaaaggaaatgccaggacaagtactaggaatgagctggagacaaagactggcatgttaacaaacattaccgccaacctacaaagacaactaaattattccgtaggaaaaaaatgaaagtgtaagatttggcagcattcaacatctaggacatacacagttgctatcgtaaattcagttttgtcgcttcggtgcttcctcaaatattttcattgaagacgaggatagcctaaacccagtgtccaattaatttacaggggaagtgtcaataccaggtccagtttacatttgttcagagtggttgactgagcttggtgctttgttgcggcaaggcccaattttctcatttggcctagggatgcatagaacaacatgcaagcccacatgggctggagcttatggagcggtatgcaggtcctggaatgtgtgctgaaaatatacgttcttcttatatatttgtattaagttgggatgttataagctttccatgtttcataattgtttatgtttgtattgacttatgtgtgtgtgtgtgtgtgtgtcccaaaaggtgtgttgtttctgtaaaaatgtagaataagtgaaaaaatggggaaaatattatagaaaagctgtatcatttattaatcccagaatgattcaagatcaacttttattacttacatgtctatgtgtaactaaagggttgacttaatttcaaaggcgattttattattgtatatatttttgaacaacacctcttcaattgcagattgccatgccaataattgagatggtactttgggtatagttcacaaaggaagggattgttatagttgactaatttctttgctccatttcactgttgtgccaaggccatccactgaatatggtagccaggactaccaccactattgccatggaggatgaagaggaagattgctccattatccttgatcttttcaaggtgtaagggcatctccaaggcggaccctcaaacctttcgcaagcgtccggaccgcactgtccaggagcagtttgccatccaacgtggtcctatatcggtccgccgagtggtccagagcatgcttttccggctaactggaaccaaacattggggggggtgcgggagtccgaatatccgtctggccaatgaaaaggcaccgtgcaccatctctactctgcgtccccactcagaagactttatactaattgtgttacatttggcagaaggtagtgcggaaggtgtttatgcatttggtggaaggtactcattgtgcttgattaggcggttgtttctagattaggtggaacttagtaacttcatttggcgaaagctagctttccgccaaatgcttatgagtagcatttggcagaagctagcttccaccaaatgcttatgagtatcatttagaggaaactagtagcttagctctgaaacagattacaaatcatagttgctattttttaaaggcaaacatttgcggtatagggttgaatggatggctcccttatcctgtttcaagacaccggtcgggacggattttcggacaaataccatgtccttctgcgggtcggcgttggagatgccctaaaggtgtacaacagacttgtcgggtcggacggtttgctgctttcctgggcttaccaataacctcatagtggtctatacatatacacacagagagaaagatttccgataagttaggcacttgctagctactcctgcggtaaagattcttcttgatatagtatgatgactccaatggtccaagtcatccaacaccagtcagcatatagcagtggcagtgccatggattcaaaccttgtgttgtcaagctttacaaatgaaatattttgtagataaacgaaacaggaacacaaaagatcatactttaattcaaacatatttgttgtagcagcctttttcaaatcatacatattttatcttcaacatgtttttatatataaaagatttaaacatgtttatggtacaacacttttgacaaatgtatccatctaccattattaaacttaagacctcaaagcaaggttttgctaaaagaaacaagatctgaatttcgtaatacgagaatagaaaattgcaagagaaagaagccagaaacttacaaacagaaggtgaagggccttgtcctaaatagtcgacggtagctggaggtgcagtactacaatgagtcagagactgccggagcagcggaggaggccgtgctcacggtggtgattccgggcagcggccacctcggcggttggtgagcagcatgtgcatccatgcatgctttgctccactgctcctccctacggcgtagtgcccaattggccattagatgaaaaaggagagaggctgaaggaaaggaatagtcacgggatttgaggagatgattaggaaattacgaagacgattaggaaacttaataatgacgtctgaagcttccttttgctatttggccagccaaatctcacgcaaaactgatacgaactgggcgtgtgttagttaatttccagaacctgtttattttgtcgtggggtacgaagtaacggactcaaggtagtcttccatactaaattcctgtgtagtcgcttgaccacacagttatagtgtggattcgccagtgcatatagggacatttggaaaaaaattgcattctgaaagtaacgctttgatagtagctccaacagtacatttgtttccacgatacactaattaagtggcccccatagactttctagtacttttcaagaggaaagggttcacatgtgtccatagactattgagtgggctccctttgtacctgttagcgcatttaacctccttttttgtcttgcaggggcatataacatgctaactaacctatatgtctaatggtcaatttaacatattaacctccatgtcaagcttcatattgcatatatgtcgttcattctgtgcgttattttatcatagggaacgatggtactatttcgtaaaaataatttggattattaataaaaataattagtgggatgtcggtgcaaatatgcgaaccactgcgctagctaaccatataagagaaactgagcagaaacagagattgttgccgttgggcgcagaaagtagtgtgttgaccctacgaacaattcatttagagaattccaccctatattaaatggaatcaaatagttgtggaacaccaaggcctaactacgaattaacaaataatacgtgatgagcgtctggacgataaattaattgttaactagtctatgggcttctatatttgatctaatcttctattgattgagtctcacatcattgaattcctttacgccagctctaatttcctctcgggcgaaacatggccatgttggaaatgttctctacatggaataacggaattttattatgctattttcatgttcatgattatctttatacttcaatgctataattgcaatggttctagaatatgagattggaaggaaaactcagttgcatgtgggaaaacaaaaacacgaagtgaatgcctagtctctcctctaaaactagctcatacattgtagatgatcatgttttcctgattatgggcattgacatgccgacaataagaagaacacattgttggttgaacaattttgttggatagacacaaattatgattaacagcgagatcacatcgtaacttagttattagaccatgtaatatcataggccttcatgccggatggttaatttggtgttgccaaacgctttccgcaatagagagatcataaaggtagcttacgggtacactgaaatagtacaccaagcttattcatgattcaaaattggggttcgcacctccaacgatggagagatactctccggtgctactctgtactacgacatccattatcatctggtcagatgtaagcgtgatatcggtcactggaatatcagaatacgaaaatgaggaaagtgagcaaaaccggtaacactagtacatcagtggggtaagcaaaatgactaaagtgagcaaagtggccactagttttttgctattagcagtgtgtcttagaactagcatgccgtagctatgctggcaaattattggatcgacgttgtttgtgccgcgacaagagtctcacgtgccactaataaagtatttattggtggtatgtcactgcaagggacgccactacaaagtggaaccccaaaacaggggccgacatatgctactggtggtgtggagtattatttaaagaaactatcgagatacaaggagaacttggatatatttgtagagaaactatttattcttaaaataacttttagataccattttcgaaattggtttaaattatgtgctcggtagagcccggtctcacgctctctctctgtctctctctctctctctctgtgtgtgtgtatctctctctcttctattgggccacaagatgccgagaggtgtgactggtagccaaaactggtttttcatccatgcgcgttcgtcattttcgtttcaaaagaaaggacaaatcaacctagtcttctccagtctatgcaaggaaggttctactgtatatttttccgtgctacaaggctgctaataattgactcggtctagtcgctggctctttctgctgccacttccattctttgagattttatctaagcatctactttaaatattccacatctgtaactagcacctcagcagttccaccctgtgagaagagcagggacagtaagctctcagttcttcacctcctctctcgtctctctttttcagttattcattaattcccaagtttgaggtggtacgtactaattcctctttatccctaccgcccagatcccttcttggatctcttctgtccagatctcaaccacacagttttgagatggtacgtactaattcctctttatctcttcaacacagtctactgtaattcctttttgtttgacagccgaaagtagtagcggccgagatcaagaacaccactacagcatttttttgttgataatttggtttacgtttgttaacacatgggaagcagccatcactttgttgaacaatctcttagtatattatagatgctggcaaaaactaagtttgaaaacggaaaacatacacgatgctggtaactgccaaaactaagtcagttgatcccctgaatatagatttgttagatgtctagcttgaacaaagtaaacctacaaatatattgtgacctcaagaacttccttatcttatatgcataattatgcgataaccattgtattggagaaagtctgctttttgtccccgaactaaaaataatggttttgggttcttaaattgaaaaaccctgccaatttggctcttgctttatgaaaccctctcaattccgttgatggtgatatgacaagggttttacacacatgcaacccagtcaaaaaagtgaatttaatgaaactaaattatctgtgcatccaaagacaagaaactacagaaattactaaaataaaagaaactattatagtaatataggaaatacattaaaaaaatataaccaaagaagaaattttagaatcaacctggcaaactaggtaattagagcaaactttgaaataatttctgaacattaacttcaaaagaaacttttgattatcatttatgtatatgtactattttaggtattctcaaatttgctagtttcatagacaaataatgtttaaaaaaattcgaaatgttcggattgcagttttttctccaaaattcaaacaagcataaatatattatttagcttctcattttttaattttttatttaatattttaatatacttttcagtagattttttcactctaaacatatttttacttgttgctaattacttggccatgacgtcatgttgggggttctaacattgatttaggatggactattgaaccgttcccgtggattggcggaaggagtggcggttatgatatgcccaatgcttctcgcacttgctctaaacatagttgacctcaatgatgaagtgtacggacatggagtccccattgccttgattgccatggcaggctttactttgatcaccggcatttgctccttcatggtgtgctgcttcatacaagggttcccgagaagtagcgtgatcctggcaatcatttcgtctgcttgcctcatcatgctcgcctacttcattgcacacttcattatcttcaaatccgtttttatcacccttggagtcctgtgtggaattttgctcatgctgcgaaccgtctgctacttgtatgatagctcaaatggcaatgttccagaatacacggccataatgcaaagcgtactggacgaatcacatgagttcttgactggcgtcactggaattctttttctggggttccaaggtttgactctggaaggtgacgttcggacaataagtcgcatgggcggggaaccaatgggttacatcagcttcatcttcatcactctaggtgtgggatcaatgctcctcgcgatgaccccacctaaaagttttggagagagaaaagttgtgcgtttgactctcctccttgactacttcatggcggctggtatcttcctgctattgttggttaccatgtgtaagcacacgcagctggagatggttgctctgttggtctttgtgcctccgattatatgcttcagttcacttctgttcctggtcttgttcccggtggacggacaagatgatgaagaccccaagccagcgtcactggaactgacaaaggtcatgttcaccggattcttggccgtttcaataagaaccatcagcaacacttcaccgaacaagctcaccggattcttcttactattatcctccatggctattgggtttggactctcatggaggctcctttcacagaacaacacaaagagtgttctcactactattgatgtttcacctgttcatgttgcttctgccgccaagattgcttccatttttacccacttttgtattgtaatcaccacaattctacttgtcgcaatggctagtactgccagcggtaaatgatcgactccagttggcaacaaaactactatgttttggttatagtactgaatatatatgatattgatctttctgcataatgtttagcaccagcacacatatgttgagtaaagtatatcaaaactattggccaagtcaagtatgtattgaaactgtaaaattggtcaatccagatactactagaagtacattaaatgagccaacacatgacttagcaaggtgaataagtcccatacaagcagatgtggcatgttagtactccctccgtttttatttacttcgcatattagctttctctgggtcaaacttcctaaatttgaccaagtttctacaaaaaaattaacatccacagtaccaagtcaataccattagaatcatcattcaatatattttcataccatacatattttttattgtgaaagtttagattgttttagatgaacttggtcaaactttataaactttgacttaggtcaaagctaatatacggataaataaaaatggagggaggaattaggcgcggaaaactcccaccaatgatgtcataaattactaaaaaattcccaaccatgcagcgaattatgatggttgaaaatagtagggctttagaccccggcctatgtatccttccatgaaatggctatttgacatgagatggcaattttatgacactggtgtcacatcgtctccctattggcctttgtagaaactgtgaaattggtcaatacagatacttgcatgcatccggcttattgctcggttggcctttgtagaacttatggtgaccctttgctgatatgctttctaaattagattgaatattctcatgaagtatttggcaaacccatttaacaaaataaaaaatagtgtggtcagcagccacgacgtttcacatcgttgagggatcctacaccactgctgcagtaggtgttctgcttgtgggcactagatgcacatacacgtcccttctcacgaagaggatacctccaccgtttccatggacggcggcctatttccatcaaaatcgccttggtgtgcgctgcatgccctgatccctctgaaggggcggcactagctgatttgccgccggtagttttggcttgttcccactcctaaccgtggcggagcaccaagctggagttttggacgatgacgacgacatgtcacgactcctctgggctcgattcttctgaggagggaggagtggccaatccaatggtttgacaatccaattagtaatttgaccaagtttctacaaaaaaattaacatccacagtaccaagtcaataccattagaatcatcattcaatatattttcataccatacatatttttattgtgaaagtttagattgttttagatgaacttggtcaaactttataaactttgacttaggtcaaagctaatatacggataaataaaaatggagggaggaattaggcgcggaaaactcccaccaatgatgtcataaattactaaaaaattcccaaccatgcagcgaattatgatggttgaaaatagtagggctttagaccccggcctatgtatccttccatgaaatggctatttgacatgagatggcaattttatgacactggtgtcacatcgtctccctattggcctttgtagaaactgtgaaattggtcaatacagatacttgcatgcatccggcttattgctcggttggcctttgtagaacttatggtgaccctttgctgatatgctttctaaattagattgaatattctcatgaagtatttggcaaacccatttaacaaaataaaaaatagtgtggtcagcagccacgacgtttcacatcgttgagggatcctacaccactgctgcagtaggtgttctgcttgtgggcactagatgcacatacacgtcccttctcacgaagaggatacctccaccgtttccatggacggcggcctatttccatcaaaatcgccttggtgtgcgctgcatgccctgatccctctgaaggggcggcactagctgatttgccgccggtagttttggcttgttcccactcctaaccgtggcggagcaccaagctggagttttggacgatgacgacgacatgtcacgactcctctgggctcgattcttctgaggagggaggagtggccaatccaatggtttgacaattgggttacgacgtcaaacaacagccggaaaaccggtggctactaggattggctcccttaccatcgctccttagcagggccctgtgtttattctcgcaagctgagccatgtccacagcattgtaagcgtacaagaggtgcccaatgatccgggcctacgtcagatggatctggagaaggggctttctggggaagaaaaggggtggtagcgggtatatcccggggggggtaggttactaccgttcgatccaataggatcggatgtttgtattggccaatcggtttgaaggcagggtggaccaatcaaaatggaggatgggcctagtgctgaatccataaccatgtttgtcgactatacatacaagaacattcggctatgcctttagaaagatatgttttacacttcttctttatttgcttatttttctttacttctttatttctttgtttaaatcgttgtttccttgtttacttacttctttgtttttatttttatttcctttcacttattttattttatttcttgtatttttctttcctgtttttttagctaggggctctccactcctctcacccctgttaaatttgaattatatccgttcaatgacaagaattttacataatggcttaaatatagcaaatgatacccccaaaaataccaaattttgacttggaatatgtgatgctcatgtttagtgtagaaatagtttcaagttcaaacgatatggagcaatcaacacctcaccttaaaacctgactcagtccttctcgaaatcacaattctaataatttttttgaaaagttcatcgaagtccctaggaatcatataatgatcgcatatgccaagtttcaagttatccagactttgtttcctgttgtcagaattaagaaaacaaataacctcatgttagaggtcgattctttccacccttgtgtttgaaattccttttgtttcttggataaggtttaaacatagactcattacataaattggattttcagcaactttttaaggcacttgcagttcaaatttgaaatgtatccattaaatgccttgaaatgcactttttgcaaggtaagagttggtttatatcagatctatcataggattacaacaactatgttggacagatacaagatcttgtggggagtatcccagccataccagaagttttaacataatgtaccaaggcgtcactaagagcattctgattcctactaatcttgtcacatttaatctcttgatcaccaaaaacatcacggatatcttgaatgtttccagcatgcattgaaagcatcttcccggatgtaatttcgttcactgcttgagcagagtctgactcaacaattatgttccaccattgtagggcgagcaggagaccctcatgtaaagcttctaaatcagctttgaaagcacatgtgcagttacatagacttaggcatgcaatgaaagtgatgttcccctactagtctggaaggtctatactagaccccaccgataaatctacatgcatataagatccgtaaatgtttagttccgccgagccggcttctggcccacgcaagttagacatccatcattcacatttttcctgtatacacttgatcgaaaggctcccgggcatgctctcattttatttgaatcgtctaaatataccatgtacggttttatccttctaatcaactaatgtctttatgtacagctaaatcatgtttgaagaaccaaccgcctatgacgtgtatgacgtccagttcttgtgcttacttcttacacaagcctgagaaaaagatcttctgttcctgcgattttttcctctaaaaataaatataagttgagcatgtcttttatcatacgcgcatccagctggttccagtatcgatcgtacggtgtggatgctgaccatggattctatagattgagatccctgagtaggacaagatccgccgttatcatcaagctgccaacctttcgttttgataaagcattatcatcgtaccccaaacattgtgcaaattttagaaacaaaaacttaaaaatatgtggttacctgtgactagaaagcggaggttctgattggccagttggtccaaacgtaacatcaccatgtacgggaaacgacatgtaaagacaaaggaaaatgccggggcaagtgctaggaatgagctggatacaaacactgggacattaagaaaaatcacttctgccgcgtctatgaagaccactaaattgttccattggagaaaatgacagttaagaaatggacagcattcagcatccgggacgtacgaatagctgtgattcgttcagttttctcgatttggtgcttcctcgaatcttcttcattgaagatcagcataaactaaacccaaggtccaattaatttacagtgggaagttcagcgccggcacccatggaacaacatgcaagcctacatgtgcttaagcccatggaacggcatgcaaatccaggagagtgtgcgtgtgtgccggtaccaccagtacggtgattcagctcccggtacagcgcccgtatgaaaagaatggccatgacaggcaagatggcgagccagatgaagccttatctagatgacaacaattgtccgcaacgcctgccctgatctgggcggtggctgtccggctgttgttcggggcctggccgttaacttgagaaaggcaatatatatatatatatttttttgaaaagatgataggcaatagttgcggcgttgtgacatcaacggcggaggtacaaagcggtgatgtgatacagggggatgttacagctattttttttcgagggtacgcaaattgcgtaccttggctttataaaagaagagaaataagacatacaacaacgtctaccactcgttgcgaacaacgaaggcgaccaactccacacccggttcgaacaaggacaaccaaacacaacaacacagctacgacacaaaaagcctacccaacaccaagcccctcgccgcgtctcggccgacattgaagacctccgaaggacagcaactccagcttccttggaatagccagcgacgaccgtacatggcgctggaggagaaagatccggacagcggtgaacaccgaaggagcgcatcatgggacctcgagtctcccagcacaatgctcccaacagagtaacaacctcaaacacattcccatcatgctgatcctacgaatcaaggctttcgccccgagacaactgccgatacgaggtcccgaggaagatggcgttgcactcgacgaagcctccagaaggtgaatgacacccgcaggtgccatcaacgccggtccggccacaaccgaacaggattttcatccgtagcgctgcacgtctccacgtctccaagatcccggccagtcccgaacagatgcctcgcaccgccttcatcgcaacaacttgccatcggagcctccctcactgctgagggaacacgcataaagccatcaccttcaaaatcgactaggagccgcagcaaccaccgagcggtgcaaagccagttccgccacggcggccttcacccgcgccaggggactgccaccagaatggatccgacttgcacctctgaccacacgccggcacccacaccgtcgaggcatcgccgcacgcctcacactgcagcaaccgaatgcccgctcccagccaagacccttccggcaaggcctcgcagcgccgccgccgccatggctgtgctcgcaccgccgccaccgccagtgccggtgcaccacctacaccctccaacctgctacgctccacacaaggatccggagacaaccgccgcactccttgccgtgagcaccacgctggccagcgccgaagacgcaccagatccggaccgagatccccggatccgcgctccgggcgccgcccgccagcaccactgacccgccgctagccaaccaccgccgggagcgagtcgcagcagagccacgggaacaccacgccacgcctaacagaggtcacagccccgaaccccgccgcccagatccatccaaggtggatccgggtcccgggctgcaccagccgtcgaggcagcaccaccggcagcaggcgccccaccacctccggcgccgagcagggacgccaccgtgccctccgccgcgccacgccgccaaggcaccagcggccagatctgagccgtcgttgggggagggaggggagctgtgccggggagatgccccgccgccgccggcactgccgtggctttgccccgcagcagaccactatggcggcgagggagggaggcgggaaagggggcggcggtggcggggcggatagggtcccccgtgtcgcctcagagagggttgcgggggttgcgggaaaacgtcaagggaatttgcgagcaagcgaaggaacgaaaactaacaacaatgatggatgttgcagctatggcttgctacatctaaggttccgtcgacctctaccaaagttgctgtagtgcacgggcacgcgaggtgtttttgctctcatgttgatgttttactctctgcatggtgaggcacttcgtgtcttcatgcatgtctttgattgcttaatgatatccaagccaaggttatgtatgcatatgagcggatgtctagaccgaaccaaacaaaaaaactgtccaaatttattttatttcagttttcggtatcagtttctgtttcgaaatttggaaaccatttggcatttggtttttggttaaaaaccaaaaacaatttggttaaaccaaattaaccaaaacagagaaaaagtaaaattatactcgcactgtcagaccaatggagcacgtgaaaaataattgtctgggcatcagggatttttgctgcaggtaatagacgaagtgggccgaagggataagtgagggggtccctttagggcccacaagccaccagggcgtgcctagcggggtgggcgcgccccggtgtctcgtgggcactggggcatccacacggggtgttttcagtgtcaaaaattcttaaatattcagtacaaattcatgaaaaatttcatatcattttgagaacttcaattttgggcacttttttattgcatggataatttggaaaacaggataatcatggtgttttattgcatttaactaaaaataacagaaactataagtgggtatagaagattgtacttactaaatttatcgaccacatgtcttaaaaaaatccattaataaggttgatcaagtcttatattaacaaaccatttttgattaacatgaaaccgaagaatcttcacaaaacattaagttacctcaacggggatatgcatctccccaattataagtgtttttgatagtaggaagaggaatttgaaaactttgaatagtaattgtcggagccctttcactaatatgaataccatttacctgaaattgttttttcgaaaagtgcaccattcgttaattaccattgatatgaaaagtgaaattgcttttgtttaatcaataatagcctctgcagtatttaaaaagggtctatcaaggataatcaacatgttgtcgtcctcgggcatatctagtataacaaagtttgttaagatagtaatatttgcaactacaacgagcacatactcacaaatactgataggtatggcagttgatttgtcagccatttgcaataatatttcagtgggagtgagtttattcaaatcatgtcttttatataaagagaaaggcataacactaacaccggctcctaaatcacacaaagcagttttaacatatttcttttaatggagcatggtatagttggtaatccggatctcctagtttcttaggtactccatcctttaaaaatagcttgcaagcatgatggagatttcagctttaggtgtttttattttattagtgacaatatccttcatatctttagtataaggggccatatttaaaatatcagtcaaacgagtacgcaaaaagactgatctaagcatttcagcaaagcgtttaaattcttcctcatccttattcttagattgtaacgactcggttccgaaaaggatccaagtctcgatgcttaatgtgccatgcccggatcggtatgctagcacacagagtactcgatgaaataacaaaaaatcacatgtctataataaagcaaggggcgtttctttgatttttctcatccgttcactgttgaattttttttttctatctgaggtggtactaaatttttgggcacctgtctgttttttagcattccagaatttcgtactttccccgcatacgttgtggcccagtgaagccagccaacttatttgcctgcccatgtagctgcaaaaagcaaatttctcgccctgtgcggtagatagttggagttcacacagggcgccaaagaatgggccgacccgttttgtttttctgtccaactttatttttatttttgcattattttgtaattcaaattttacaaaacaattcagaatttgaatgttttatcaaatttttgaaaaatgttcggattgcagtattttcttcccttttcaaaaatgtttggaacatcaatttattttctcgttccacctttttacataatcagaaatattcaaaaagttgtttgtgatataaaaaacatgtattttaaaaatattcgaaatttgaaatatattcttattttacttaaatgctcacagattaaaaataatgtctatgtttaaaaacacattttataatattgttctgaatgttcaaaacatgtttcgttttcaaaagtccgtcacaaattaaagaaaatgttttgggtttttataaaaggttcatgtcttcaaaaaatgatgttggttcaaattttgttcatgtttttttcttccaaaatggacggaatattcaaaacattgttcgtaatttcaataaatgttcacgtttccaagaatatccggtaattcatatttttagagaagttcatttaaaaaaatctaaggttccactatgttcttaaatattcacaattgtccattggtttgtagtagtcatctgttgtcgtggctaggggcacgtggtcgggtctttaaggtcatgtgtttgaccattcaacacatcttttttaggattattactcatgacttgcaaacacaggttatttccgtgcctgccagtgggctggcccaaacgggaactgttgtgcatctcacgcgttatttgacacaactagcttcatatagtagctcccacgctactgccctacgaaaagaaaaatatatacgtatagtttctgttccgtcccttccacacgtaactccccagttttttcggttacctctcgagtgggccaaactggacttaccacatatttcttttttttttgtcggacacacgagtagcaaaaaataaagggtgctagggtattgaactatggaccttccctgttatgcaattatgataggaacagtcccatcttgaaatttttatatgtctttaagttgtctcaaaataaggaggttgtgtgagaataaggaagttgtctcgagaattatgagcagaacgatggactccacaaattattgcctgaggtaacttgcacgtgcaaataaaagacaatatgccggtcggctataatggaaagaaattgtgcacgtgctaataaaagagaatatgctgagtttactctaattaataaaatttatgggcacgtgagcgcaaaaaaagatgaagagaataaatcctgataatgaagggacattcatgcttggttatgcgctgattatgctcatgaaacataatttatgcgttgcaattagtaatccatttggttacatcgtcgtaggagaaagtggtcgaagcgaccatgcctcgaaggtcgccacatagaaacctaatgaggacgcaaccagaactctgagtcggaggtggacccggaggaaaaaaaaagtaccagttgggtggtcgccgccacgcggaccggccccaccactatcaagatctcgcgggccaaagatgacccatgagaagaccgaaactcctcgctctgtcacccactgccgtggagaccgtttttgatgaacacatcagttatctctcccgctgcttgttgcaaaaacttatcttcccgtcgcaatgcacgggcatatatatgtgctagtataaaataacgtaaatgttattaccttaatccatacaatggaaatatcaacagaggttgtggagtcccatcaacatcaacggcaaagttgagtgtagacaacataaccctatcgtgtcacttactcgtcatataaatcctccaacacgagatgttacagccgtgtaggttagtacgttgaatgtactgacaagtcacaccacaagagagtatgaaagtcctacttctacatgcaatttggctggtggaagattctaagtttaatttgcataaagctgatttttctctatcatcaaaaagaatatcaaactattactataacgtttaccataatatgatatatatggttcctccaatcaacatatatccaaattaattaaaacccattcaataagttaaatgtgatgggatcgctcaaattcccaatttctatatgctcaaattgtccataatcggggacacggctaatcaattagtttgacactctgcgaaggtttgcgcacttttcccccacaagactcgatcacctccttggccgtcctcgcactgcccggtgtttgagaaccagaagatcgagacatagtctttcagaagcattccccttaaccctgtgtagaccagtacacctacatatataactacatatgctagcctacctgaaaagaggccacgcaacgtactcaactaagccagaggccatattagcttgtggctgcgcaggtaagcttctagtaatgaaatattgatgaatatctttgagcccggatggcattccatagggtgatctcacaggtcctccggtatccccttgggcaagcattggtttctctaggagcacaggacaaaccactgattactacaaggtgcacctacccatccacccagatgtgtgttaatgttgccatcttaggttacccaaaattattatacctctcgcaacttgcatgaatcacacataccaccccgtagcatagcaaaaatttctacaacgttggcgaaatggagtaaagtctcagacttacctcagtgaactattgggtttaaaatatagccaacttatatatatatatatacattcatgcaatcctacatcaaaggactaaatgcatataaaaaacttagttattaagaggcatgatcaagatgtaacttgccttgtactgttggtgaagattcttcacactcacagttcttgataacgatactattcacactccgacacataagcaatcactcacagatccaaaagaaaaaggcaaagaaagattgcgaaaactccaaaagaaccaaatcaaattattttacaagattagatcctaattttgccgaaaaagtgtggtgatggctaaagatggttgattccgtctagggatttcaatttgcaaaaggaattaattaaaaatgcactgcagtttacacaatatgatcaaatgaaggtccaaatttaaatttgaaaggttcaaaagtttaaactaatgatgccactggatgtaggaagtcaatatgaacatgtagcaaaaagaattactagattggaaataacggatcaaaagatacaagtaattcaacatagcattgaatctgcttagaatgttgtgtgacaactctttggtggactagtagagtaatctgactatgatcaaaattatatgttgaactgaacagaaagatttatttgatacgaatacgatgcaattggattcatcgaaaacggagctacggataaaaagatatattcaaaagctgtttgaatatgaatctgaacaaatttcaaaattttggaagttgcataaaaattgatttaatagtttcactgggtaggttggatcaagacgaaaaatataggcattggtctcatctaatttggatgagcgagtaaaaagttataactatctgaaaatcagggccaaactgttttacggaagaaaaagagctatggctaaaagaattctagggctaatgtgtaaatatagagactaattaataatctaactattttacatataaaaccttacgtggaacaaaataaatagattgtgataggtttctgccataaatatgaagagagtacctttagagtgccataaatacgcaggtaagccaaggttatgcatacgagcggatgtctatgggcaccctacacatatacctaaatagttgatcccaactatcacaattttctcatcatgcacatatgccactccatcatacatgccatatcatcgcctcattaattatttctctaaacatgtatgggaaatgctatttatattctctcttaacacacacatcccaactcacgacacatgttacctcatcaatcaaactaattatatttctctcaacaggcttgtcaacctctacttatattctatatatgttcagaaatgcataatgcttaaatactatatatcatatttattttaatgttcatatgaatttcaccaaaattgcaaggccaaaactgttgaagaattttcgtcccattagtctttgcaaggtgttgtttaagatagcctcaaaagtgttggctaaccatctcaaagtgttgctgctggaggtgatctttgactatcaaagtgcttttgtacccgggagattgattactgagaatgctttggttgcctatgagtgtatgcacattatctgacaagaacatgctaagcaagccttctttgccatgaagattgatatgatgaaatcatacgttcgtgttgaatggaattatcttccggttgtctttcaaaacttggctttgctccagcttggatagaaactgtcatgcattttccgacaagatcttgcgcaggttagatatcaacaactagctctttattttctgcattctaaaatttgaccgcagaaaatttgtcttttagcaatgcaatcatatcatgcattgatactgctagcctacataatatgtgctgttcttagaaacagtggtcttggtaattttgtatcatgctcatgtagaaactttttcccttcttttaaccaaaggtgcggttcgccgttcaagaacaacatctgaaatttacctgtttttccttatacaattttgtgagagttgcaaagattcttgcgggaagtatctgctattgaacatataaatatgtgctcttgggctgttcttatccactaaaagaccacgcatttcctacttcgtatcgctgaaacagttttacccggatggctaggtgtacaatacccttggggtgagctacaagcgcgagaacaaactggacaagacaatcaagcaattcgagaaggccatggagctccgaccaggctaggtgacagcgtggaacaacctcggcgatgcatatgagctgtagaaggacctcgcgtctgcactcaaggcattcgaggaggtattgctctttgatccgaaaaacaaggtggcaaggccgcgccgagatgatctcaagtcacgtgttggcatgtagaagggcgtgccagtgaagacgacggacaagcggtagccgccacagatggaggaattagtgcctcagctttgaacgttagcatttgtaagcatgataagttgtatgcagcatattggtgtcgtcatacgtctgcgctggtggttgtagaatgattgaatgttagcacaaaatgtatacttttatttcgagaatcgcaccaagtccataacagttattgcaacgttactcttgcacttgctatttttttctgaacaccgacagtgttctgtttacaaacagtattcgtttcggcacatttgcagaaacgaagacgacatggtagaaaatcaattaattctatcgataaggtagcaatttgtgcactaccactgttgtcaagttttcccaatttgtgtgtatgttctctaccacccctacggttcttttataggtaggctcaagacgagcccggttttgagttaacaaatccatcaattcgtactccctctgtaaagaaatataagacttattatatttccttacagagaagtacaaaatatagggcagctaaacgagcttacaacacaacacacaaatgacagacaccaaaactgaacagatggtgttgcccttcacgctcgaacacccgactacacacatgacaaacaacatcgacagagcaactagatgctacgtactccctccattccatattagttgtcgccgctttagtacaaacagagagagagtatgagagcgagcatgcccggtgatagcatgaggaatagagaagaccaagcacactcctcgcttgccaaagagggaacctccccctcatccgggctcgatgacgtcgcactgttattcatgcaagcaagataattcgtaccacaagatcacatcacacatagttagagtacagctgaatatggaaaccacgagggaaccagcagaggaaggagcctacgactaggagaatctcctatcgatgacggccttggcctttcccttgccttcaggcttgcccttctcttcgggtttgctctttggcggcggcttctagcccttgagattcttcccggacgggacggccttctccaccgctggcgccggggatgctgctgcttcttgcccaaggggctccaccttgggggcgccatggatgatacagcttccaaacgtgtggtgtggagatggggtggcttgtatctatacgtacctacctttttttttcatgaaatggaacacaaacgattccatacttcacacaggtccagccatagtgtcatcacggagatcctcagaccagtctctccacttgtacctacctactaataaaaagcccctaatttttttctttaactaatccgtggtccaacattaagtgccctttttctgttgggtttcggcccaactgcggcagccactatctcgcgcgtctggtcgccggcgcgccagttagtattacgcgttcagctgcagtagaccaagttcgctttgttttcagcccattgggacccggctttcattatttacatagaatatgttaagcataattatgctataactttataaccataactcggataaaaataattatatacatgttatttgaccataaaatgcgtagtttgcaaccgtgctattatttgttcctgttagccactttaaaattttattcagggcataaagttaattaacctaattaattgctatggtctattttggaaaatgctaacacacaatataacttttatatcatctctatatatatgaaatatgttacgagggcctataaatcatagggactatgaatgaccaatatcagtgggcatataggagtaattaacaaataagagaaaagaaggcgtgatcagtagggatagaaaaaagaacgggagattcaaaacaacatgaatgagagaaaagacaaatgaattggtattaataccaagggcgggcacgcaatgaaataataaaaaggaattaataccgagggacgtaggaggggcatgcatatgtgtgttgttgcgcagtgtgcgacctatgtgcaatagaaataagagcgtcgaattgtatgcaacgaactagggtcatttcgctcagaagtctaatttttttgtattaatcttcaagtgtatgcagagatggcctaaaactgatttaacagaatccttctattgagatcagaatcgccgtataaaaatcgtagcaaaagtattgtatgtttaccatgcttcttaatgtaatactgatataagtttctgctagatcttagtgtgctcctctctgccatagccgggcacaattcagatgttgttgggacatgctaacggatatctaagtggacacgtcaactatctctcctgttgcaatgcacgggcatgtgtgctagttttcgctcaaaaaggcttctccaagcaccacatatatcctgtaaaccttatcggtaccaaccgcatgaacttatatttctctctcttcattctttaacgtccatagcaaagcaattagcggagatcgaatcacaaagaagaagaaataactttggtcgtttatcacctgcatcttcttggaccaaccatgtgctctctatctctatcatgctcaccggtcacactagtagaaaaccacccattagtccggttcgtaagggcctttagtcccggttcatgaaccgggactaatgggtcgttactaatgccaccacccattagtcctggttcaaacacgaaccgggaccaatatggctccacgtggtcctgtgcaccgagcccagtcagagggcctttggtcccggttggtggctccaaccgggaccaaaagtcaatttttttacgaaagtggctgctttaggggttttgggggttatttttaggttgttattatagctagctaatagagagaagtgttctctcttatcttcgtccttggtttatcaacgctgctgctctgttcattgcacccgcagatatgacatgctcatgcatgctttgcatcatacatcatcatatatatgtatataataaagtcctactaatcatgcatcatcatacaacttctactcgttattaatcataagtcatacgatcatcatcctcatatatagtcatcgaacccaatcctacataattgttcttagcacatgatcatcagtatcaggtataggacctaaacacccttaaggtaaaatagcataaaataatatagaccctaactctccattatgaagaatggcgatcatcctgtctccaattcttgcctttcgctgcttattgcttccaagaagctccttacgactgtccatacatttttttccattctttgattgttatgtctccacttcttttagaaacccggtatggacagttgagattcgtaggacgacccgggtagtatgttcaaaacatgaaggctaccacacgtatacatcagatgaggcacacaatcattcgggattatctgttgaaaaacatagtaataacttcgtagttagcaatgatgtactagttttagaagtgtgcaaaaagatgcacggatgttgtaatagtaaaaaataatcttaccaaggtatctccatggtagttaccgtagttcaacacgtgcactagtggcacgtattgaccataatgttgaggagtttgattgtagatattgtaattctcaagatcagtacaaaatccgaccagatgttttttctcctgataagttaattcggagccttcggtgtagtacgttctgtctaccatcttccgcacattttttgaagaatgaaaataagctgtcaatggagataagttgtcaactatatattttgaaataaacaatataaattacttaataactatgtttagctcacatgagggaagaactggaggtgtatccacaaagacccaaatcgaaggtgtctcttgcttgattgtaggatcaccaagatccatggtgacaatcataccctcatcaagaccatacatcttgcaaaatgcatcccaatttttgcaaccaaaatgggttacactctgagcattgtacagctttacttgaaaatccacaccatgttcggtacttaggataatttttttggtttcgaaattttcatggtcttcaaaatccatcctctccaagacatagcgtcttgcaaagcatgggataagctagtcgaattggaaaagatgaaaaatacacgttaaaatagttgaagtcgtgcttaattatgaaaaaaaatattgtcgtcgttgcgtaccgtatgaacatcccaagtctcctcgagcttaatgctgaagcgccgatcttcgtccagctcaatgactctgtcgcacatacctcggtcgtcgtggcaccagttgcactccctcgggcgattttcgtcgtccgatgagtacgacatttccggcctacgttcataattcaaatattaaactagatcattattattaatcacgggttgactatcgatgatcgatgtacgtacgtagctcctctttcattcccgaatgcattattataccaaattgtctagcacacgggaatgaaagagaacttatccaatatgagcattcaataaacaaaaccaaatcacgaaataaagtatatatagtattcaaattagcatgcattcaataattataagcaaaagtacatcatctcttggtgtccgtacatcatcgaatattatcactaatacaactagaaccgtagcgcccgacaggtatcgacgcgggcggtcgacacccaaagataaggaaccatcacagtatcatagctccagtgagatccctgaagaacctgccggtattgtcgaacctcccctccaatgcaaccatgtagcgacggacgtgctcgtcctcctcgctgacacggtgacgtaccacctccgcggtgtccggatgcctcaccaccgtcactggcccacgcgaccgccaccaaacaaggatcgggtcaacaacgggctgcctcctcaccaacctacgtcccccggaaggtagcacctcccaataccaacccggcggagcccagtcccgaacatgaccctgatcaagcaggcctccaccgccgagtcgacgatgacgacgatccgggataggcatcgccgacgttgacgcgggaactacttcgatatatagttaaataaaagtagtcttattaattaaatcaattatctagttcaactactaaacacttactataaataaataagtagtacttactaaaaacaaactacttcattctatagtaaaataaattagtttattaaatcagctagctagttccactatatataaactacttcttctttttttttcctttttctaaatactatgaacaaaaaaatcattaaaattactacacataatataatacatatctaaattactacacatcttctaacaaaaagatctaaattacacaatatgaacaaatctaacagaaaaaaatctaacataaaaaaatctaacataaaaaatctatttacagaaaaaaatctaacataatataatacatatctaaattactacacatctaatctaacaaaaagatctaaattacacaatatgaacaaatctaacgaaaaaaatctaacataaaaaaactaacataaaaaatctatttacggaaaaaaatctaacataatataatacatatctaaattactacacatctaatctaacaaaaaaatctaacataatatgaacaaattaattacacaatctacattaactacatatctaaattactacacatctaatctaaccaaaaaaatctatgaactacaaaaaaatctaaaaacaaatctagcaaaaattaaaaaaagaggctcacaggggcgacggcgatggctcggtgcggcgcggggcggcgacggcgtcgaggcggcgcgggctggggcggctgcaggggcgggcggcgacggcgcacggggcggcatggggcggcgacggcgtcgaggcggggcggggcggggcggggtggcgacggcgaggtagcggcaggggacgcggcgacggcggaggaggagaggccggagagatcgaagtcgggagaggaggacgctcagtgttgtatatatagcaatggcattggtcccggttcttggcacaaaccgggaccaatgcctttagtcccggttgatgcctccaaccgggaccaaaggcctcttttcagcagcccaaagggcgggaaacgaagacctttggtcccggttggtggctccaaccgggacaaaagaagggcattggtcccggttggaggcaccaaccgggaccaaagagaggcattggtcccggttggtgccaccaaccggtaccaatggacccgtctaactacttttttatcttctgcagctttttcttgttgattcttcctgcagctgttgttttagtcccacctcgccaagcgagaggaactcgcagctgtttataagccctgagtgcagatacaatgacgaagaggctcaatgctcctgcacgttggttagcttcaagccttgcggaatacggtagactacacagggctatgtgcagtgcagttgacactattccgaaaggcttgaagcaaattaacgagcattgcgcctcttttttatttttaatgacttgttacaactcagaaataaaaaaaatgtagcagaaaagaaaaaaatatataaaaaccactcagaaataaatagaagaaaaaaaagaaaatagttgtgattaactgtactaaaaaggagcatatatgcttatttttaatggcttatcacaacacagaaataaaaagaaaaaataatatagcagaaaaagaaaaaaaaaactatatagaaaactactcagaaatgaatagaagcaaaaaatagttgtgattaactatactaaaaaggagcatatatgcttatttttaatggcttattacaactcagaaataaaaagaaaaataatatagcaagaaaaagaaaaaaaactatataaaaaactactcagaaatgaatagagcaaaaaatagttgtgattaactatactaaaaaggagcatatatgcttatttttaatggcttattacaactcagaaataaaaagaaaaaataatatatcagaaaaagaaaaaaactatataaaaacaactcataaatgaatagaagcaaaaaatagttgtgattaactatactaaaaacggagcatatatgcttatttttaatggcttattacaactcagaaataataagaaaaaataaatatagaagaaaaagaaaaaaactactcagaaattagtacagatgcgcttatagaggaaattcaacttaaattcataacgaatttcaagagaaatccgtatgaatttatgctaaattacctatataagcgcatctattttcattttcggaggagctcaatagggtagagagggaggggcttataaactggtcccgttccccttcggttggcgaggtgggactaaactctgatcgcaacgggtaccaaccatttagtcccggttgattgctcgaaccgggactaatggaagcccattggtcccggttcaagtcgccaaccgagaccaatggttttgggccaggagcgaggaccattggtcccggttcgtcccatcaaccgggaccaataggtttagacgaaccgggaccaatggcccacgtggcccggccggcccccggggctcacgaaccgggtccgatgcccccattggtctctgttctggattgaaccgggactaatgggctgggccggcctggaccattggcccgttttctactagtgtcatgacctagtcattttttcttgaaatagaggatagccccctgcctccgtattaattgatccacacatctatatattattgattattcaacaatgtcaagcaaaaccaagcattaatgtcaatcaaaacggcttacagaacaataaagaaaaaccggtggtcgttagcctcaccgtagaaactccataatcctaccccaaagtctatgacatgtgaccgaaaaacaaaaatatactactcattgacaaagcctttggaaaagaaccgccacactagcaccgatgttgacgagttcaaccataagacaaaacatgggatttggacatagaattttgatccccaaaatggagctttagaaccaacccctttaacaagatcacgacatgcccacatcaataattctggtctaaccagacagacaagattgtggattttcacccggactaaacgcattgccgtcagaatcgtcactgtgagcaacccctctactggctgtcaaacaatgaaccggtaggtccatatggacttgggcaaggaagcacaataatgcctcatgattgtcccactcggaacaccaagacaatgtaaactccgggttatgttgacgaactttgtgtttgtcgattatctatacttgttcttgtgcttgtgtgcttgagaacttgtgaactacgaacttgtgcttgtgactttctgatgtatgttcaggaattgtggtgtggttatgttttagtatttttaaatatgtgagatgtgataaatgttgaatataattgtcacatatatattattgtagggctaaatgcaaaacaagtttttaaaatccgttgatcgaacctttgtcaagtgttacactcgacaaaggtatgtatgccgagtgcaacaatcggcgaaggtgccatgtggctcaacctagtaactcctcgaagcttttgccgagctccttacagagacactctataaaggatgccacatgacacaccccggcactgtgcgagccagagaataggtgagatactcggcaaagagtatgcatctttgcccagtgtcggcactcggcgaactggtgggacacgtgtcgccctccaatgcatcgaccttcgctgagcatcggtactaagcaaagagtatgtttctttaccgagtgtcggcactcggcacactggagggacacatggcatcctcatatgcaccgtgcttcgtcgagcgcttctcaagcggtactccaaacgtttgtgccatgtggctttacccttttcatttgtcaaccgtcaagaccctttattttcccgagtaccctgtcctgacactcgacaaagggtttgccgagtgctcgacggaagcaatttgcggctgcattcacactcgaacgattttaatttctgcagaaagtctgacgtgatgctctcaaattgccatttgccgactgcagaaagtctgacgtcatgctctcgaattaccattaagtcaactgcggctgcgttcacactcgaacgattttaatttcttaatctaacttttggtgattcaacagtttcggctcattgtccattaggcaaaagatttcacccggaattagcaaaatccgagaccataacatcgtgggcacacacacacacagagacaaactaaacaagtcgatcccacattcaattcggatcgagcaggcagttctccatgtacataatgataattaacaacaaagtcttgcagcagcttccggggtcttccggtgcggcgtgctcgccgctgacgtctcttgtggtggtggcggcggcggatcgaccggttcctcgccgggccgattcggctgccggggcttaggggacggaggagcagccggcacgcggattccgcgagaagcacctgcgcataatcatgatcctgtgagctgcaagcaattaagcacgtgcgcatgcgtatacgaagcctagcgtagacggcaccatggacgtaccttgggccgccgccgccgccgagcttgttgccgggaacagcatatgaggagagccaccagtgccaaggaagaagaagagcgctgcatgattcgccctgtctcagcttctgaatgagtactagtatatggctgttctcctccagtgtacgtacgtagttttatatagggaggtgatctggagcagattggaagacaaattctcttttggttcctgcactggtcacagccgtcccggtcaagagggttttgggtactcatctaaacaatcagtgcgtgtgtgctgtgtgtgaatttgcgacgccgcgtctccagaatttgcatggcgcgtttacttttttgagagagtcatggcacgtttacttgcatgctacaggaacatgtttttgtcacatttacacagggaaggaacaacaatgttgatttcaccctaaaaaatatcctagaagaattctaaaggccaagtaaaccatttttttttggttttctccgtccccggcccgcctagtcttcggtggccttatgataaatgtgtgcgtgatgtaccatcttcgcaccagagacttccggtccccactctgacaaggatctaaagagaaatatgaacttgctgcagcaacttggtatacttccttgtggtgctacggcatggttgagtttcgcacagtcagcacagtctagtgaaggagtaaagttaaatatggtttacgaggttggggagctgtttgagctgggaattcagctttcgtatctgctcatactgcttggtctgcttggaactggtacattttttgtcatccgccggttcttgtccgcagggagcttgatctttctgcgaaagacttgcaggtatgcatcctgctaacatttctacatgcattcagactaccatgggtcaatattatccctctgactttgttcttcaaacctgtcgacatgagttcttgataacatcgtactgtaattactgaatactctgaagtattaaacttcagtttaatgtagagattatcaatgatggcgacactgattgtgatgtaacatcacatttttactattgttattatgttaaatttttgactgtacattttcagtagtttgctgaatttgtcttgcttgatatagtttagttaggcaagagcattatctaggcgagtgacgtgcaggcatgtaaacatatttttcaaagaaatttgtattgtagtgaaatcatctaacaaaaaaggtaaattctcctcatttagaaaattgttagtctcagttttctccttgaattaccagagttattgttgatgacccggctgacatccgacctctatcaaaggaacttcctaacatggcatctcgtctacaatatttgttcccgggatcttacattatttagtgtgaataattcttatggtttcctcaagcctaagtcatttttgttacttccatggaacgtatgggcattcaactagtcttataaaaaatatgatcttgaagtctggacatccctttcttgtggtcaatctttccaattttttgtttcagaaatgagaaagcacttggttagcgctgccttgttgtattcattacatgcgcaaatccccatatcatggtagcgcaggtccaatccttgtactgaccagttaatttgacttcatcgaagttctttattgatgttgctcgcttcataataaatgttgtattcgcacgctatgtcttgtgtggtgtcgatgaatgtggtgtacttacgtcaaccatacttgattatcttaattataggaacaagtgagaagtcgtgagggaagcgctactgaactttttgagcttggagcagtcatgctccggagaaagttttaccctgcagctatcaaatatctacagcaagcaatccaaaaatgggatagagatgagcaagatcttgcacaggttagatatccacaacaactagctagtctccctccgttcacaaatataagatgttttggatatttcaatatggactacatacgggctgaaatgagtgaacatacacactaaaacttatctatatacagccgattcatgattttttttagagcatcttatatttgtgaacggagggagtatttgttttctgcattctaaaatctgtccgcaggaaatttaccttttaccaatgcaatgatatcatgcattgatactgctagcctacataatacacgctgttcttagaaacagtgttgctggtaatttggtatcatgttcatttagaaactttttccttcttttaaccaaaggcgccgtttgccgttcaagaactacatctgaaatttacctatttttcttgtacagttttgtgagaattgatatagattcttgcggcaagtatatgccattgagcctatatatttgtgctcttgggctgtgcttatccactaaaaaccaatatgaccacccatttcctatttcgtctcggtgaaacagtattacctcgatggccaggtgtagaatgccttgggggtgagctacaagcgggagaacaaactggacaaggcaatcaagcaattcgagaaggccgtggagctccaaccaggctatgtgacggcatggaacaacctcggcgatgcatatgagcagcagaaggaccttgcctcagcgctcagggccttcgaggaggtattgctctttgatccaaacaacaaggtggcaaggccacgccgagatgacctcaagtcgcgtgtgggcatgtacaagggggtgccagtgaagacaacggacaagcgatagcctagccgccacggatggacggattcgcggtttcctcagttttgaacagcacgtagcatctgttgtagcatctgtaaacatgatgagaagttctatgtagcatattggtgtgatccatccagagctggctgttgtagaatgtttcgaatgttagcacaaaaaaaatgtagaatttgctttcgagatccgcaccaagtctgtaacaggaggatgacggcggcgtggcgcagtggagtctcagcgtcggatgtgtgtacatggactcgtgcaggaggatggcgctgtatggtgtcatggtggcatggacggcagagaggcctggcaaagttggtgcattgatctctccggaagatgggacggtggaagatggtggcgacagagcctagagcgtgcgcatgctgtgcacgctgagcgtctgtttcagccggttggtactcttgaatcttgattcgcaggcacttcatcaagctggtcggtatagccgacggaggcatcaaataggtggctttggatggatccatgtatttgttttgtcagactttgtggaataagttaataaaaatggttgtgtgcatcctttggtgtagaggccggggtttcaacctccctttggaaaaaaatcagtccatcagaaattacaggatgtagggaagctaaacgagcttacaacgcaccacacagatggcagacaccaaaactgaacagatggtgttgcccttcacgctcaaacaccaaactacagacatgacaaacaacatcgatggtggggtttaaggtctggatcgagctggaagggatcccgccccatgccgggatcattctgttgtggaagatttgctgggatcgtcgtgcaaaattgacaccgtcgcgccggagaccgcctctcgggccagccggattcgttcaagacttcggcgtggacggtggacccgcagagcatccccaccctggctggctcgccgtgccggagccacggtgaggagcggccgccgacgttcctgcagtacaaggtgcttattcacttagacgcggtgatggatctccgggaagctggtgagccggtgttcttcgatggctcatctgacagcagccagagtggcattccggagcaagatagagacttgggagggccaggccctaggccttgcaggatgccgtggcagttcggggtccgcgactcgcgtggtgctggcggagtcgccggcggcagtggtggcgggtgtgtccatggggttgccggcggcgggatggccaggatggtgcctccacttctgattggtgcctcccccaatgggcacgacgtgcgatgcaggtccgggtcccacaaggatgatggctgcgaacaggattacacgcaggcagtcggcttttgaccgtctgaccacgaccgcgggacgggggaacgttcggatgcgatttcaagcgaggcaagtgggagagatgctccaaggactgggccagtgctgctgatggtctcgccaaggcgtggagtggccccggatacgctgtccaatcagcggaagatggtgacctcggatccgctgtctacccgggagggggaaccgaggtggatgcatcacgggacccggggacaactaacctgctgctacggtcggagggcggggggtgcgcggaatcaggacgagccattggccgcccgggatccaaggcaggtcgcgacaatgggcgggccaccaccgtgctgtctgctcgactgtcaggcagaagatcaggtgggtcctacagagcaggtgggacgccggggagaaagcgacaaagaggatcgggctgtgctggaggattaggagaggatgatggacccagaggagtgagacccgaggggcgggtgtagttgggcggggtggtggagacagttaaccccgccaccacgattaccgagacccaaccaccaagggaggatggggtctacccagacaaaaatcatgcacatgcaacgtccgggatggctacatcgatcgcatgcggaagagaggggccgggccttgttgaggagatcgcggtgacggccaacgcttgcaaaacgatgatatgcgctcctagggaccagaaactgaagggatgactgctaatgaggcggtggcatacagcaagctcaagagtttttgctccaatatcatcaaacgcctcgccccaccgctgctctgggaagtgcaggcctcccaactacagcctgaggctgagcctttcacgcccaagaggaccataagagcatccaagaagggcacggcgatcaagaggccgcatgacacgccagtggagaatgtgcttctccacgcacttggcatggtgccgggagatcttgaggtggatgatggggtggtgcacgagctcaaggagctcttcgactctccgctgcgcgaacagcagttacggatcattgcggctctcttcggcaagtcgttacctttggtggatgaaatggaggcacaaaatgcggtggtggtgtgcacttaggcggagtgccttgcaaagttagtgtgatcttttccatggatccacaccagaaagtgctatgctggaccgttcgcgggctgaacaatccagctaaacacaaggcggtgcgcgagttcgtgagtacattagatgttaatctcatttgttttcaagagaccaagatggatgtaatagatagttttacagtgatgcagtgcatggggccatcctttgatggctttgcctacttgccgccggaggagacgtgtgggggtgtgttgctagcttgggattgctcggttctggatgtggaggccatccaacttgactcaaacttcatcacgggcagggtgcgaactaaagttggtgaaccatggtggatctcggtggtttatggcccccaaggagacgagcggaagagccagttcttggatgagctcagactgaggcgaacggtctgccccggtgcttggctagtgttaggtgattccaacatgattctgcgtgcctccgagaagagcaacaataatcttaataagagcatgatgaacaagttcaaagacttcgttgacgatcatgagctttgggagatgtacatgcacagtaggaagtatacttggtccaatgaaagggatgcacccacgctgaccaagatcgatcgtgtcctcatgacggtggactgggagctcgaaaatgcgaaccacctgctcgggtgctttccacgggagtctcagaccatgcacctctccttctcagcacgagtgcaggtttttgccccaaaaacgcttcagattcgagcttttctagaccaggcttgagggtttcgaggaggcggtgtgcgaggcatggttctgtgatcccgggattgtggatccctacaaaggttggacgcgttgctgcgcaactcggcgatctcgctccaaacatgggaccaacgtaaggtcgggaatgtgaagctactgatgcggtttgccacttggctgatatagaggctcgacatcgcgcaggagaacgggattttgtgtgtcagggaggtttggctgcgacgcactctcaaccaagcgctgccggcatggcggcccctggagagaacgattgacgggcagcggtcccgagtgagatggatcagagaaggagacgccaacactaagcttttcaggcagtggccaatggtagaaggaccaaaaacttcatcggtcagatcaaagtgggggacaatgtcatcacgaatcaggacaggaaaatggaggcgttcacagaggcttatgaaaggctgctgggaaccgcgcaaactaggaagcatacactcaacttgcattacttagggattgaagcgagggagctgagcaacttagaagtgatgatcacggaggaggaggtttggcaggttatcagagaaatgcctccggatagggccccggggccggatggctacattggagccttcttccaaaaggcgtggcaaatcattaagcaagatgctatggccgtggtgatgaagctctttgtgggggacacgagaggatttgccaagatgaacaaagcaaacatcatcctcattcctaagaagcccgacgctgacgagattggcgattacaggccaataagtctaatacatagcatcccgaagttattcgccaagatcccgccaaccgactaagactccaaatgaaggagctggtagctatgaaccagtgggcgttcatcaagggccgacacctgcatgacaacttcttattggtaagacaagtggcaaggaaaattcatgcgagaagaaattcggacgtcttcctgaagctggatatatcgagagcttttgactccctctcatggtccttcctcttcgagatgttgagagtgaagggcttcggtcaaagatggttaacctggatggcgacgctcttgatgactgctagcactaaggtggtggtgaacggcgtggcaggaagattgtgcatgcctgtggtttgaggcaaggagacccgatatctcctatgctcttcgttattgccatggaaggcctctcggccctcatgcacaaggctatggaagtgggcgcactcaatgccttcactgggatcagcccaatgcagagcgtctccatatatgctgacgatgtggccatgttcactaagcctacatcgcaagatctggcgacagtccgagctattctggccacgtttggagaggccttgggcctcaaggtcaactaccacaagtctacagtggtgatcatcagaggtacgtacgatgacaagactagggtggcaagcatgctccagtgcaacacctctgatttcccctgcaaatatctggggttgcaactcgcaattaagagcctaacgcgcgcggactggcagccgatgttggacaaggtgcgccactgcttgccgggttggcagagggggctgattcagaggcccggcaggttaaccttggtgcagtctgtcgtcgcagctcggccggtgcaccacctgatggtcttggaggcgccggagtgggtccttgaggacatgaacaaatggatgagatcttttttctgggccggcaaggacaagtgcaatggaggacagtgcccggtagcttgggaaaaacaatttcgccccaaaactgttgggggctgggcatcaagaatctaaagtggcaggcgatcgcgcttagagttagatgggagtggctcaagcggaccgatacccatcgcccgtggcaaggcctgccgatgatgagagacgaggaggctaaagcggtgttcgacaccttggtcgacagcaaagtcggatcgggcgagcgggtgctcttctggaaggatagatggattcacggggcagcggctgcggacattgcaccattgctggtcgccttggtcccagccaggacggcaaatagacgcacggtgtgtcaggccctcgacggtgctagctggacacaggacttcgaggccaacacatccttcatggccctcattcagctcatgcaccttgcgcacgtgataaacacggtgccaagggatccggaccagcctgacagcttcactggccgctaatgacgtcaggctcctactcggccaaatcggtctatcaatacctttgctcggagctgaccagatcgccaacggcaagctgcacttggaggacttgggcaccgctcaaatgcaaactttttgtctggatagctttgcagaaccgtttgtggacatctgataggagagccaggcacgggcttcaagaccaaccgtcgacttgctatacgtgcttgcaggaggaggacaatgtggcacacatcccggctaactgcacctacgctcaggaggtgtggcacagggtgtttggccttctccacctcaacatacatgggccactggagacggtcgagttcacggagtggtggcttaccgcgcatacaagcttctttagggctgatcgaagagggtttgatacgacggtgactgcggttgcatggcctttgtggaagcagaggaatgccagagtcttcaacaaggtcaatgagcaaaagactgcgtttgatcttccattcataatcctcgacgaaatcgcggagtggaagatggcgggtttgggcgtaggtgggctacaatgttttgtgagaagttagcatagcgagtgtggagttggagttgtgttcgttttgtgtccttcgtgttcgcacctaggacacaatcttgtaaatacttttctctcttctataaagctatggtatgccattggcatactctcgaaaaaaaacatcgaaggagcaactagatgctactccatccgttccatattagttgtcgtcgattcagtacaaacggagagggagagggtatgagagcgagcacgccggttatagcatcagccaaagagggaacctccccctggtggtccgggctcgaggatgccgcaccgttgttcacgcaagcagcataattcatactactagaagaaaacatcacacatagtcagagtagatctgaggagcataggaaggagcctacgactaggagaatttcctatcattgacggcctggggcttccccttgccttcgggctttcccttctcctcgggcttgcccttccctcgggattgcccttgggccgcggcttctcgcccttgagctccttcccggatggggtggccttctccaccgcgggcgccatggatgctgctgcttccatacgtgtggtgtggtagtgtggagatgtggcgacttgtcttttctcttagaaaggcttctccaagcaccacatatatcctgtaaaccttatctctaccaaccgcatgacctctatttctctctctcactctctctcactctccattcgttaatgtccacagcaaagattgatcggccatcgagtcacaaagaagaagaaatgacttcggctgtttgtcacctgcatcttttgggacactagtcacaaccttgtcatttttcttgatctacaatgaatgtttcttttggaacactgtcggtagggtgctccaagcccataacagccaatttttgcacttgctaatttttctgaacaccgacagtgttctgtttacaaacaacattcttttgggcacatttgcagcaacgaagaccacattgctaacaaataaattatttctatcgatagcgtagcagcttatgctaacaaataaataagttttgcccaattttaatgccccttcactacccttatcgttttttagaaggtaggatagctcaatacgaatccccatttgaatcaacgaagtcattaacctgtcaggaattacaagatacagaatgctgatacccagagcttacaacacaatacacacgcaaaaaattgaaagcagcagcttaacttgcagatggtttcggcctccatgctctaaggagcaaccgggagtagatgttacgagagcgggcacgcctctgaatctgtgatagcatcaggagtatagaagacaaaccacgagggaaccagtagagaatttcgcccctgccttcagccgtggcttctcacccttgagatccttcccggacagggtgggggggcagccttctccatcacgggcgccttgtcatctttaaccttctattaatgctcaatcaaatttttcttcagttgcttatgagttgctcgatgctgaatttgttgatgcatttgaacaaactcatcaaacatggccggattctgatactgaacttcgatagtcacccatattcttaaattcaagagttgcgccagcatcctcaccctcgtcattaatgatcatgctatgcatgatcatacaacatatcattacctcccacaaggtcttcagatacgtccacaaaaactgcaaaacatgccctctcaacatcctttctaacttcttgtattgggcaaagtgagaccttctctcaccatgtggctctgagatggtcttgccaaggtagcccacatacgatagataccatcaaccacatcgtagcacatattttattcatgcccattgaaatcatagtcgcaatgccgagctttcctcggtcagcctagcaaacaatggatattgctgcaaaccgacgagaaaattct

SEQ ID NO: 72 - DNA, 18 bp, Artificial Sequence
  Oilgonucleotide primer
tgattgcttcatggcggg

SEQ ID NO: 73 - DNA, 19 bp, Artificial Sequence
  Oilgonucleotide primer
gggcaaccatctcaagctg

SEQ ID NO: 74 - DNA, 24 bp, Artificial Sequence
  Oligonucleotide primer
ctcagccttaacctcgaccgcccg

SEQ ID NO: 75 - DNA, 35 bp, Artificial Sequence
  Oligonucleotide primer
tacacctagggcaatagtgagtcccttcatgtgtc

SEQ ID NO: 76 - DNA, 23 bp, Artificial Sequence
  Oligonucleotide primer
tgcagcatctattcatatgctct

SEQ ID NO: 77 - DNA, 24 bp, Artificial Sequence
  Oligonucleotide primer
aaagagagtcgagatagacttacc

SEQ ID NO: 78 - DNA, 20 bp, Artificial Sequence
  Oligonucleotide primer
tgcttctcgcacttgctcta

SEQ ID NO: 79 - DNA, 19 bp, Artificial Sequence
  Oligonucleotide primer
aagcagcacaccatgaagg

SEQ ID NO: 80 - DNA, 21 bp, Artificial Sequence
  Oligonucleotide primer
tccccattgccttgattgcca

SEQ ID NO: 81 - DNA, 57 bp, Artificial Sequence
  Oligonucleotide primer
ggggacaagtttgtacaaaaaagcaggcttaatggtacgtactaattcctctttatc

SEQ ID NO: 82 - DNA, 56 bp, Artificial Sequence
  Oligonucleotide primer
ggggaccactttgtacaagaaagctgggtctttaccgctggcagtactagccattg

SEQ ID NO: 83 - DNA, 55 bp, Artificial Sequence
  Oligonucleotide primer
ggggacaagtttgtacaaaaaagcaggcttaatggagtggagccaaggatctcag

SEQ ID NO: 84 - DNA, 55 bp, Artificial Sequence
  Oligonucleotide primer
ggggaccactttgtacaagaaagctgggtctagtggttgttggagatcaagccgg

SEQ ID NO: 85 - protein, 374 aa, Triticum aestivum cv Chinese Spring; identical to SEQ ID NO: 1

SEQ ID NO: 86 - protein, 378 aa, Triticum aestivum cv Chinese Spring
MEWSQGSQQGKILSDAQTPPVPSGLPLVGFACLPEGVAFEEPLSLSLYCQEMVGVDVVFHYDGTSKDTSELLTGTIEVAAYFYYLSTLAPSNLFLARHLAHHGHCFHKALYIKLNHHTGDSHGKVTPGSSNIDEKTIKTPMELRIIVVATFGALLVMDQLDDDTAGGFTISQFLLFLSSTVAALTCMVMKLPADRFLGSAPASELLQKTLLLLLLVTVHTLAAELLGEDVVLFCMPEVIPVLLWFSLNLDRPHHSPLTNIYKMKRHLKGLTIALGVLVVVPLFAYLVSSMDVSGLSYCTTIMVACGISGVLTYYIVFMLNYWHWPWQKQQAAAGSSSKDDVPSSCMLKFWADVLLIVAAVLLLLRYMVDVRLDLQQPL